(19) United States
(12) Patent Application Publication
(10) Pub. No.: US 2024/0067712 A1
(43) Pub. Date: Feb. 29, 2024

(54) ANTIBODIES TO HUMAN GDF8

(71) Applicant: Regeneron Pharmaceuticals, Inc., Tarrytown, NY (US)

(72) Inventors: Trevor Stitt, Ridgewood, NJ (US); Esther Latres, New York, NY (US)

(21) Appl. No.: 18/045,588

(22) Filed: Oct. 11, 2022

Related U.S. Application Data
(63) Continuation of application No. 15/860,200, filed on Jan. 2, 2018, now abandoned.
Application No. 15/860,200 is a continuation of application No. 14/974,147, filed on Dec. 18, 2015, now Pat. No. 9,890,212.
Application No. 14/974,147 is a continuation of application No. 14/462,085, filed on Aug. 18, 2014, now Pat. No. 9,260,515.
Application No. 14/462,085 is a continuation of application No. 13/115,170, filed on May 25, 2011, now Pat. No. 8,840,894.
(60) Provisional application No. 61/348,559, filed on May 26, 2010.
(60) Provisional application No. 61/372,882, filed on Aug. 12, 2010.

(51) Int. Cl. C07K 16/22; A61K 39/395

(52) U.S. Cl. CPC C07K 16/22; A61K 39/3955; A61K 2039/505

(57) ABSTRACT

The present invention provides isolated human or humanized antibodies or antigen-binding fragments thereof which specifically bind to Growth and Differentiation Factor-8 (GDF8) and block GDF8 activity. The antibodies and antibody fragments of the present invention may be used in therapeutic methods for treating conditions or disorders which are ameliorated or improved by inhibition of GDF8.

### CROSS-REFERENCE TO RELATED APPLICATIONS

This application is a continuation of U.S. patent application Ser. No. 15/860,200, filed Jan. 2, 2018, which is a continuation of U.S. patent application Ser. No. 14/974,147, filed Dec. 18, 2015, now U.S. Pat. No. 9,890,212, issued Feb. 13, 2018, which is a continuation of U.S. patent application Ser. No. 14/462,085, filed Aug. 18, 2014, now U.S. Pat. No. 9,260,515, issued Feb. 16, 2016; which is a continuation of U.S. patent application Ser. No. 13/115,170, filed May 25, 2011, now U.S. Pat. No. 8,840,894, issued Sep. 23, 2014, which claims the benefit under 35 U.S.C. § 119(e) of U.S. provisional application No. 61/348,559, filed on May 26, 2010; and 61/372,882, filed on Aug. 12, 2010, the disclosures of each of which are herein incorporated by reference in their entireties.

### REFERENCE TO A SEQUENCE LISTING

The instant application contains a Sequence Listing, which has been submitted electronically in XML format and is hereby incorporated by reference in its entirety. Said XML file, created on Oct. 7, 2022, is named Sequence-Listing-40848-0012USC4.xml and is 458,159 bytes in size.

### FIELD OF THE INVENTION

The present invention relates to antibodies, and antigen-binding fragments thereof, which are specific for growth and differentiation factor-8 (GDF8).

### BACKGROUND

Growth and Differentiation Factor-8 (GDF8), also known as myostatin, is a member of the TGF-β superfamily of growth factors. GDF8 is a negative regulator of skeletal muscle mass, highly expressed in the developing and adult skeletal muscle.

GDF8 is highly conserved across species, and the amino acid sequences of murine and human GDF8 are identical (human GDF8 nucleic acid sequence and amino acid sequence shown in SEQ ID NO:338-339) (McPherron et al. 1977 Nature 387:83-90).

A number of human diseases are associated with loss or impairment of muscle tissue, for example, muscular dystrophy, muscle atrophy, muscle wasting syndrome, sarcopenia and cachexia, and inhibitors of GDF8 are applicable to treating these diseases or disorders.

Antibodies to GDF8 and therapeutic methods are disclosed in, e.g., U.S. Pat. Nos. 6,096,506, 7,320,789, 7,807,159, WO 2007/047112, WO 2005/094446, US 2007/0087000, U.S. Pat. No. 7,261,893, and WO 2010/070094.

### BRIEF SUMMARY OF THE INVENTION

The present invention provides human or humanized antibodies and antigen-binding fragments of human or humanized antibodies that specifically bind human growth and differentiation factor 8 (GDF8). These antibodies are characterized by binding to GDF8 with high affinity and by the ability to neutralize GDF8 activity. The antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al. (2000) J. Immunol. 164:1925-1933).

In one embodiment, the antibody of the invention comprises a heavy chain variable region (HCVR) amino acid sequence selected from the group consisting of SEQ ID NO:2, 18, 34, 50, 66, 82, 98, 114, 130, 146, 162, 178, 194, 210, 226, 242, 258, 274, 290, 306, 360, and 376, or a substantially identical sequence thereof.

In one embodiment, the antibody of the invention comprises a light chain variable region (LCVR) amino acid sequence selected from the group consisting of SEQ ID NO:10, 26, 42, 58, 74, 90, 106, 122, 138, 154, 170, 186, 202, 218, 234, 250, 266, 282, 298, 314, 322, 368, and 384 or a substantially identical sequence thereof.

In one embodiment, the antibody of the invention comprises a HCVR amino acid sequence and a LCVR amino acid sequence, wherein the HCVR/LCVR pair sequences are selected from the group consisting of SEQ ID NO:2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 114/322, 360/368, and 376/384.

The present invention also features a human or humanized antibody or antigen-binding fragment of an antibody comprising a heavy chain complementarity determining region 3 (HCDR3) amino acid sequence and a light chain CDR3 amino acid sequence (LCDR3), wherein the HCDR3 amino acid sequence is selected from the group consisting of SEQ ID NO:8, 24, 40, 56, 72, 88, 104, 120, 136, 152, 168, 184, 200, 216, 232, 248, 264, 280, 296, 312, 366, and 382, or a substantially identical sequence thereof, and the LCDR3 amino acid sequence is selected from the group consisting of SEQ ID NO:16, 32, 48, 64, 80, 96, 112, 128, 144, 160, 176, 192, 208, 224, 240, 256, 272, 288, 304, 320, 328, 374, and 390, or a substantially identical sequence thereof. In another embodiment, the antibody or fragment thereof comprises an HCDR3/LCDR3 amino acid sequence pair selected from the group consisting of SEQ ID NO:8/16, 24/32, 40/48, 56/64, 72/80, 88/96, 104/112, 120/128, 136/144, 152/160, 168/176, 184/192, 200/208, 216/224, 232/240, 248/256, 264/272, 280/288, 296/304, 312/320, 120/328, 366/374, and 382/390.

In a related embodiment, the antibody or fragment thereof further comprises heavy chain CDR1 (HCDR1) and CDR2 (HCDR2) amino acid sequences and light chain CDR1 (LCDR1) and CDR2 (LCDR2) amino acid sequences, wherein the HCDR1 amino acid sequence is selected from the group consisting of SEQ ID NO:4, 20, 36, 52, 68, 84, 100, 116, 132, 148, 164, 180, 196, 212, 228, 244, 260, 276, 292, 308, 362, and 378, or a substantially identical sequence thereof; the HCDR2 amino acid sequence is selected from the group consisting of SEQ ID NO:6, 22, 38, 54, 70, 86, 102, 118, 134, 150, 166, 182, 198, 214, 230, 246, 262, 278, 294, 310, 364, and 380, or a substantially identical sequence thereof; the LCDR1 amino acid sequence is selected from the group consisting of SEQ ID NO:12, 28, 44, 60, 76, 92, 108, 124, 140, 156, 172, 188, 204, 220, 236, 252, 268, 284, 300, 316, 324, 370, and 386 or a substantially identical sequence thereof; and the LCDR2 amino acid sequence is selected from the group consisting of SEQ ID NO:14 (Gly Val Ser), 30 (Ala Ala Ser), 46 (Ala Ala Ser), 62 (Ala Ala Ser), 78 (Ala Ala Ser), 94 (Ala Ala Ser), 110 (Ala Ala Ser), 126 (Arg Met Ser), 142 (Val Ala Ser), 158 (Ala Ala Ser), 174 (Gly Ala Ser), 190 (Ala Thr Ser), 206 (Gly Ala Ser), 222 (Gly Ala Ser), 238 (Gly Ala Ser), 254 (Gly Ala Ser), 270 (Leu Gly Ser), 286 (Gly Ala Ser), 302 (Ala Ala Ser), 318 (Leu Gly Ser), 326 (Lys Ile Ser), 372 (Thr Thr Ser), and 388 (Ala Ala Ser), or a substantially identical sequence thereof. In another embodiment, the HCDR1, HCDR2 and HCDR3 are selected from the group consisting of SEQ ID NO:36/38/40, 116/118/120, 228/230/232, 362/364/366, and 378/380/382; and LCDR1, LCDR2 and LCDR3 are selected from the group consisting of SEQ ID NO:44/46/48, 124/126/128, 236/238/240, 370/372/374, and 386/388/390. In yet another embodiment, the heavy and light chain CDRs are selected from the group consisting of SEQ ID NO: 36/38/40/44/46/48 (e.g. 21-E5), 116/118/120/124/126/128 (e.g. 8D12), 228/230/232/236/238/240 (e.g. 1A2), 362/364/366/370/372/374 (e.g. H4H1657N2), and 378/380/382/386/388/390 (e.g. H4H1669P).

In a related embodiment, the invention includes an antibody or antigen-binding fragment of an antibody which specifically binds GDF8, wherein the antibody or fragment comprises the heavy and light chain CDR domains contained within heavy and light chain variable domain sequences selected from the group consisting of SEQ ID NO: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 114/322, 360/368, and 376/384. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The present invention also provides nucleic acid molecules encoding the antibodies or antigen-binding fragments of the invention. Recombinant expression vectors carrying the antibody-encoding nucleic acids of the invention, and host cells into which such vectors have been introduced, are also encompassed by the invention, as are methods of making the antibodies of the invention by culturing the host cells of the invention.

In one embodiment, the antibody of the invention comprises a HCVR encoded by a nucleotide sequence selected from the group consisting of SEQ ID NO:1, 17, 33, 49, 65, 81, 97, 113, 129, 145, 161, 177, 193, 209, 225, 241, 257, 273, 289, 305, 359, and 375, or a substantially similar sequence having at least 95% homology thereof.

In one embodiment, the antibody of the invention comprises a LCVR encoded by a nucleotide sequence selected from the group consisting of SEQ ID NO:9, 25, 41, 57, 73, 89, 105, 121, 137, 153, 169, 185, 201, 217, 233, 249, 265, 281, 297, 313, 321, 367, and 383 or a substantially similar sequence having at least 95% homology thereof.

In one embodiment, the antibody of the invention comprises a HCVR amino acid sequence and a LCVR amino acid sequence, wherein the HCV/LCVR pair sequences are encoded by a nucleic acid molecule pair selected from the group consisting of SEQ ID NO: 1/9, 17/25, 33/41, 49/57, 65/73, 81/89, 97/105, 113/121, 129/137, 145/153, 161/169, 177/185, 193/201, 209/217, 225/233, 241/249, 257/265, 273/281, 289/297, 305/313, 113/321, 359/367, and 375/383.

The present invention also features a human or humanized antibody or antibody fragment comprising a HCDR3 encoded by a nucleotide sequence selected from the group consisting of SEQ ID NO:7, 23, 39, 55, 71, 87, 103, 119, 135, 151, 167, 183, 199, 215, 231, 247, 263, 279, 295, 311, 365, and 381, or a substantially similar sequence having at least 95% homology thereof and a LCDR3 encoded by a nucleotide sequence selected from the group consisting of SEQ ID NO:15, 31, 47, 63, 79, 95, 111, 127, 143, 159, 175, 191, 207, 223, 239, 255, 271, 287, 303, 319, 327, 373, and 389, or a substantially similar sequence having at least 95% homology thereof. In one embodiment, the HCDR3/LCDR3 set is encoded by a nucleotide sequence pair selected from the group consisting of SEQ ID NO:7/15, 23/31, 39/47, 55/63, 71/79, 87/95, 103/111, 119/127, 135/143, 151/159, 167/175, 183/191, 199/207, 215/223, 231/239, 247/255, 263/271, 279/287, 295/303, 311/319, 119/327, 365/373, and 381/389.

In a related embodiment, the antibody or antibody fragment further comprises a HCDR1 and HCDR2, and a LCDR1 and LCDR2, wherein the HCDR1 is encoded by a nucleotide sequence selected from the group consisting of SEQ ID NO:3, 19, 35, 51, 67, 83, 99, 115, 131, 147, 163, 179, 195, 211, 227, 243, 259, 275, 291, 307, 361, and 377, or a substantially similar sequence having at least 95% homology thereof, the HCDR2 is encoded by a nucleotide sequence selected from the group consisting of SEQ ID NO:5, 21, 37, 53, 69, 85, 101, 117, 133, 149, 165, 181, 197, 213, 229, 245, 261, 277, 293, 309, 363, and 379, or a substantially similar sequence having at least 95% homology thereof, the LCDR1 is encoded by a nucleotide sequence selected from the group consisting of SEQ ID NO:11, 27, 43, 59, 75, 91, 107, 123, 139, 155, 171, 187, 203, 219, 235, 251, 267, 283, 299, 315, 323, 369, and 385 or a substantially similar sequence having at least 95% homology thereof, and the LCDR2 encoded by a nucleotide sequence selected from the group consisting of SEQ ID NO:13 (ggtgtatcc), 29 (gctgcatcc), 45 (gctgcatcc), 61 (gctgcatcc), 77 (gctgcatcc), 93 (gctgcatcc), 109 (gctgcatcc), 125 (cggatgtcc), 141 (gttgcatcc), 157 (gctgcatcc), 173 (ggtgcatcc), 189 (gctacatcc), 205 (ggtgcatcc), 221 (ggtgcatcc), 237 (ggtgcatcc), 253 (ggtgcatcc), 269 (ttgggttct), 285 (ggtgcatcc), 301 (gctgcatcc), 317 (ttgggttct), 325 (aagatttct), 371 (actacatcc), and 387 (gctgcatcc), or a substantially similar sequence having at least 95% homology thereof. In one embodiment, the antibody or antibody fragment comprises heavy and light chain CDRs encoded by a nucleic acid sequence set of SEQ ID NO:35/37/39/43/45/47, 115/117/119/123/125/127, 227/229/231/235/237/239, 361/363/365/369/371/373, or 377/379/381/385/387/389.

The present invention also features an isolated antibody or antibody fragment that specifically binds GDF8, comprising heavy and light chain CDRs selected from the group consisting of (a) a HCDR1 comprising an amino acid sequence of the formula X¹-X²-X³-X⁴-X⁵-X⁶-X⁷-X⁸ (SEQ ID NO:329), wherein X¹ is Gly; X² is Phe; X³ is Thr; X⁴ is Phe; X⁵ is Ser; X⁶ is Ala or Ser; X⁷ is Phe or Tyr; X⁸ is Gly or Ala; (b) a HCDR2 comprising an amino acid sequence of the formula X¹-X²-X³-X⁴-X⁵-X⁶-X⁷-X⁸ (SEQ ID NO:330), wherein X¹ is Ile; X² Gly or Ser; X³ is Tyr or Gly; X⁴ Ser or Asp; X⁵ is Gly; X⁶ is Gly; X⁷ is Ser or Asn; and X⁸ is Ala or Glu; (c) a HCDR3 comprising an amino acid sequence of the formula X¹-X²-X³-X⁴-X⁵-X⁶-X⁷-X⁸-X⁹-X¹⁰ -X11-X¹²-X¹³-X¹⁴ (SEQ ID NO:331), wherein X¹ is Ser or Ala; X² is Thr or Lys; X³ is Asp or Ile; X⁴ is Gly or Ser; X⁵ is Ala or His; X⁶ is Trp or Tyr; X⁷ is Lys or Asp; X⁸ is Met or Ile; X⁹ is Ser or Leu; X¹⁰ is Gly or Ser; X¹¹ is Leu or Gly; X¹² is Asp or Met; X¹³ is Val or Asp; X¹⁴ is Val or absent; (d) a LCDR1 comprising an amino acid sequence of the formula X¹-X²-X³-X⁴-X⁵-X⁶ (SEQ ID NO:332), wherein X¹ is Gln; X² is Asp or Gly; X³ is Ile; X⁴ is Ser; X⁵ is Asp or Asn; and X⁶ is Tyr or Trp; (e) a LCDR2 comprising an amino acid sequence of the formula X¹-X²-X³ (SEQ ID NO:333), wherein X¹ is Thr or Ala; X² is Thr or Ala; and X³ is Ser; and (f) a LCDR3 region comprising an amino acid sequence of the formula X¹-X²-X³-X⁴-X⁵-X⁶-X⁷-X⁸-X⁹ (SEQ ID NO:334), wherein X¹ is Gln; X² is Lys or Gln; X³ is Ala or Tyr; X⁴ is Asp or Asn; X⁵ is Ser; X⁶ is Ala or Phe; X⁷ is Pro; X⁸ is Leu; and X⁹ is Thr.

The methodology for deriving the aforementioned consensus sequences (SEQ ID NOs: 329-334) is illustrated in FIGS. 4A and 4B.

The present invention also features a fully human or humanized antibody or antibody fragment which binds GDF8 with an affinity (expressed as a dissociation constant, “K_{D}”) of about 1 nM or less, as measured by surface plasmon resonance assay (for example, BIACORE™). In certain embodiments, the antibody of the invention exhibits a K_{D} of about 700 pM or less; about 500 pM or less; about 320 pM or less; about 160 pM or less; about 100 pM or less; about 50 pM or less; about 10 pM or less; or about 5 pM or less.

In one embodiment, the invention provides a fully human or humanized monoclonal antibody (mAb) which specifically binds and inhibits human GDF8 and exhibits an IC₅₀ of less than or equal to about 10 nM; about 5 nM or less; about 3 nM or less; about 2 nM or less; about 1 nM or less; about 500 pM or less; or about 200 pM or less, as measured by GDF8 inducible luciferase assay. As shown in the experimental section below, some of the anti-GDF8 antibodies of the invention block the activity of closely related proteins, such as GDF11, with a much higher IC₅₀ than GDF8 in a luciferase bioassay. In one embodiment, the invention provides an antibody or antigen-binding fragment of an antibody that exhibits at least about 10-fold, at least about 50-fold, at least about 100-fold, at least about 200-fold, at least about 500-fold, at least about 1000-fold, or at least about 1500-fold higher IC₅₀ for blocking GDF11 activity relative to GDF8.

The invention encompasses anti-GDF8 antibodies having a modified glycosylation pattern. In some applications, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of a galactosylation can be made in order to modify complement dependent cytotoxicity (CDC).

The invention includes anti-GDF8 antibodies which bind specific epitopes of GDF8 and are capable of blocking the biological activity of GDF8. In a first embodiment, the antibody of the invention binds an epitope of the mature GDF8 protein (SEQ ID NO:340) within amino acids from about 1 to about 109; from about 1 to about 54; from about 1 to about 44; from about 1 to about 34; from about 1 to about 24; and from about 1 to about 14. In a second embodiment, the antibody of the invention binds one or more of an epitope of the mature GDF8 protein (SEQ ID NO:340) within amino acids from about 35 to about 109; from about 45 to about 109; from about 55 to about 109; from about 65 to about 109; from about 75 to about 109; from about 85 to about 109; from about 92 to about 109; or from about 95 to about 109. In a third embodiment, the antibody or antigen-binding fragment of the antibody binds within an epitope of the mature human GDF8 protein from about amino acid residue 48 to about 72; from about 48 to about 69; from about 48 to about 65; from about 52 to about 72; from about 52 to about 65; or from about 56 to about 65.

In a related embodiment, the invention provides an antibody or antigen-binding fragment thereof that competes for specific binding to GDF8 with another antibody comprising a HCDR1/HCDR2/HCDR3/LCDR1/LCDR2/LCDR3 amino acid sequence combination of SEQ ID NO:36/38/40/44/46/48, 116/118/120/124/126/128, 228/230/232/236/238/240, 362/364/366/370/372/374, or 378/380/382/386/388/390. In one embodiment, the antibody or antigen-binding fragment of the invention competes for specific binding to GDF8 with another antibody comprising a HCVR/LCVR amino acid sequence pair of SEQ ID NO:2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 114/322, 360/368, or 376/384. In yet another related embodiment, the invention provides an antibody or antigen-binding fragment thereof that recognizes the epitope on GDF8 that is recognized by another antibody comprising a HCDRs/LCDRs amino acid sequence combination of SEQ ID NO: 36/38/40/44/46/48, 116/118/120/124/126/128, 228/230/232/236/238/240, 362/364/366/370/372/374, or 378/380/382/386/388/390. In one embodiment, the antibody or antigen-binding fragment of the invention recognizes the epitope on GDF8 that is recognized by another antibody comprising a HCVR/LCVR amino acid sequence pair of SEQ ID NO:2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 114/322, 360/368, or 376/384.

The present invention also features a composition comprising a recombinant human or humanized anti-human GDF8 antibody and an acceptable carrier. Further included in the invention are vectors and host cells comprising vectors which contain nucleic acid molecules encoding the human anti-GDF8 antibody of the invention, as well as methods of producing these novel antibodies, comprising growing a host cell comprising nucleic acid encoding the anti-GDF8 antibody of the invention or an antibody fragment, under conditions permitting production of the protein and recovering the protein so produced.

The present invention also features methods for inhibiting GDF8 activity using an antibody, or antigen-binding portion thereof, of the invention. In one embodiment, the method comprises administering an antibody or antibody fragment of the invention, to a human subject suffering from a disorder which is ameliorated by inhibition of GDF8 activity. In preferred embodiments, the human subject treated with the antibody or antibody fragment of the invention is in need of improving glucose homeostasis, decreasing fat mass, increasing insulin sensitivity, improving kidney function and/or decreasing fat accumulation. The antibody or antibody fragment of the invention is useful for treating, preventing or inhibiting a disease or condition characterized by bone loss, including osteoporosis, osteopenia, osteoarthritis and bone fractures, treating metabolic syndrome, counteracting muscle wasting from sustained administration of a glucocorticoid or a steroid hormone or muscle loss related to muscle dystrophy, muscle atrophy, muscle wasting syndrome, sarcopenia and cachexia.

Other objects and advantages will become apparent from a review of the ensuing detailed description.

### BRIEF DESCRIPTION OF THE FIGURES

FIG. 1. Immunoblot of Limited Proteolysis of Human GDF8 with Proteinase K. Gels were nonreducing 18% SDS-PAGE with 0.2 μg GDF8 loaded in each lane, and 2 μg/ml of antibodies either control I (A), 1A2 (B) or 21-E9 (C). Lane 1: digest time 10 min, 1 μg GDF8, 0 μg Proteinase K; Lane 2: digest time 10 min, GDF8 1 μg, 1 μg Proteinase K; Lane 3: digest time 10 min, 1 μg GDF8, 6 μg Proteinase K; Lane 4: digest time 45 min, 1 μg GDF8, 0 μg Proteinase K; Lane 5: digest time 45 min, GDF8 1 μg, 1 μg Proteinase K; Lane 6: digest time 45 min, 1 μg GDF8, 6 μg Proteinase K.

FIG. 2. Immunoblot of Limited Proteolysis of Human GDF8 with High Doses of Proteinase K. Gels were nonreducing 18% SDS-PAGE with 0 or 4 μg GDF8 loaded in each lane, and 2 μg/ml of either control I (A) or 1A2 (B). Lane 1: digest time 16 hr, 0 μg GDF8, 96 μg Proteinase K; Lane 2: digest time 16 hr, GDF8 4 μg, 0 μg Proteinase K; Lane 3: digest time 16 hr, 4 μg GDF8, 24 μg Proteinase K; Lane 4: digest time 16 hr, 4 μg GDF8, 96 μg Proteinase K; Lane 5: digest time 1 hr, GDF8 4 μg, 24 μg Proteinase K; Lane 6: digest time 1 hr, 4 μg GDF8, 96 μg Proteinase K; Lane 7: digest time 10 min, GDF8 4 μg, 0 μg Proteinase K; Lane 8: digest time 10 min, 4 μg GDF8, 24 μg Proteinase K.

FIGS. 3A and 3B. Insulin tolerance test before and after antibody treatment, respectively. Graphs illustrating the percent starting glucose levels over time in mice subjected to an insulin tolerance test before (FIG. 3A) and after (FIG. 3B) antibody treatment.

FIGS. 4A and 4B. Alignment of the amino acid sequences of the heavy chain CDRs (FIG. 4A) and light chain CDRs (FIG. 4B) from exemplary anti-GDF8 antibodies H4H1657N2 and H4H1669P, illustrating the consensus sequences shared between these sequences.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Definitions

“Human Growth Differentiation Factor-8”, “GDF8” and “myostatin” are used interchangeably to refer to the protein encoded by the nucleic acid sequence of SEQ ID NO:338 and the protein having the amino acid sequence of SEQ ID NO:339 (propeptide) and 340 (mature protein).

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-GDF8 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as diabodies, triabodies, tetrabodies and minibodies, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The antibodies of the present invention may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the invention in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al., Proc. Natl. Acad. Sci. (USA) 95:652-656 (1998)).

The term “specifically binds,” or the like, means that an antibody or antigen-binding fragment thereof forms a complex with an antigen that is relatively stable under physiologic conditions. Specific binding can be characterized by a dissociation constant of 1×10⁻⁸ M or less. Methods for determining whether two molecules specifically bind are well known in the art and include, for example, equilibrium dialysis, surface plasmon resonance, and the like. For example, an antibody that “specifically binds” human GDF8, as used in the context of the present invention, includes antibodies that bind human GDF8 or portion thereof (e.g., a peptide comprising at least 6 contiguous amino acids of SEQ ID NO:340) with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. (See, e.g., Example 3, herein). An isolated antibody that specifically binds human GDF8 may, however, have cross-reactivity to other antigens, such as GDF8 molecules from other species.

The term “high affinity” antibody refers to those antibodies capable of binding to GDF8 with a dissociation constant (K_{D}) of about 10⁻⁸ M or less, about 10⁻⁹ M or less, about 10⁻¹⁰ M or less, about 10⁻¹¹ M or less, or about 10⁻¹² M or less, as measured by surface plasmon resonance, e.g., BIACORE™ or solution-affinity ELISA.

By the term “slow off rate” or “Koff” is meant an antibody that dissociates from GDF8 with a rate constant of 1×10⁻³ s⁻¹ or less, preferably 1×10⁻⁴ s⁻¹ or less, as determined by surface plasmon resonance, e.g., BIACORE™.

A “neutralizing” or “blocking” antibody, is intended to refer to an antibody whose binding to GDF8 results in inhibition of the biological activity of GDF8. This inhibition of the biological activity of GDF8 can be assessed by measuring one or more indicators of GDF8 biological activity. These indicators of GDF8 biological activity can be assessed by one or more of several standard in vitro or in vivo assays known in the art (see examples below).

The fully-human anti-GDF8 antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are back-mutated to the corresponding germline residue(s) or to a conservative amino acid substitution (natural or non-natural) of the corresponding germline residue(s) (such sequence changes are referred to herein as “germline back-mutations”). A person of ordinary skill in the art, starting with the heavy and light chain variable region sequences disclosed herein, can easily produce numerous antibodies and antigen-binding fragments which comprise one or more individual germline back-mutations or combinations thereof. In certain embodiments, all of the framework and/or CDR residues within the V_{H} and/or V_{L} domains are mutated back to the germline sequence. In other embodiments, only certain residues are mutated back to the germline sequence, e.g., only the mutated residues found within the first 8 amino acids of FR1 or within the last 8 amino acids of FR4, or only the mutated residues found within CDR1, CDR2 or CDR3. Furthermore, the antibodies of the present invention may contain any combination of two or more germline back-mutations within the framework and/or CDR regions, i.e., wherein certain individual residues are mutated back to the germline sequence while certain other residues that differ from the germline sequence are maintained. Once obtained, antibodies and antigen-binding fragments that contain one or more germline back-mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes anti-GDF8 antibodies comprising variants of any of the HCVR, LCVR, and/or CDR amino acid sequences disclosed herein having one or more conservative substitutions. For example, the present invention includes anti-GDF8 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. In one embodiment, the antibody comprises an HCVR having an amino acid sequence selected from SEQ ID NO:360 and 376 with 8 or fewer conservative amino acid substitutions. In another embodiment, the antibody comprises an HCVR having an amino acid sequence selected from SEQ ID NO:360 and 376 with 6 or fewer conservative amino acid substitutions. In another embodiment, the antibody comprises an HCVR having an amino acid sequence selected from SEQ ID NO:360 and 376 with 4 or fewer conservative amino acid substitutions. In another embodiment, the antibody comprises an HCVR having an amino acid sequence selected from SEQ ID NO:360 and 376 with 2 or fewer conservative amino acid substitutions. In one embodiment, the antibody comprises an LCVR having an amino acid sequence selected from SEQ ID NO:368 and 384 with 8 or fewer conservative amino acid substitutions. In another embodiment, the antibody comprises an LCVR having an amino acid sequence selected from SEQ ID NO:368 and 384 with 6 or fewer conservative amino acid substitutions. In another embodiment, the antibody comprises an LCVR having an amino acid sequence selected from SEQ ID NO:368 and 384 with 4 or fewer conservative amino acid substitutions. In another embodiment, the antibody comprises an LCVR having an amino acid sequence selected from SEQ ID NO:368 and 384 with 2 or fewer conservative amino acid substitutions.

In certain embodiments, antibody or antibody fragment of the invention may be conjugated to a therapeutic moiety (“immunoconjugate”), such as a cytotoxin, a chemotherapeutic drug, and immunosuppressant or a radioisotope.

An “isolated antibody,” as used herein, means an antibody that has been identified and separated and/or recovered from at least one component of its natural environment. For example, an antibody that has been separated or removed from at least one component of an organism, tissue or cell in which the antibody naturally exists or is naturally produced is an “isolated antibody” for purposes of the present invention. An isolated antibody also includes an antibody in situ within a recombinant cell, as well as an antibody that has been subjected to at least one purification or isolation step. According to certain embodiments, an isolated antibody may be substantially free of other cellular material and/or chemicals.

The term “surface plasmon resonance”, as used herein, refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl groups, or sulfonyl groups, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. For example, an antibody is said to specifically bind an antigen when the K_{D} is less than or equal to 10⁻⁸ M, less than or equal to 10⁻⁹ M, or less than or equal to 10⁻¹⁰ M.

A protein or polypeptide is “substantially pure,” “substantially homogeneous” or “substantially purified” when at least about 60 to 75% of a sample exhibits a single species of polypeptide. The polypeptide or protein may be monomeric or multimeric. A substantially pure polypeptide or protein will typically comprise about 50%, 60, 70%, 80% or 90% w/w of a protein sample, usually about 95%, and preferably over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide analog or variant” as used herein refers to a polypeptide that is comprised of a segment of at least 25 amino acids that has substantial identity to a portion of an amino acid sequence and that has at least one of the following properties: (1) specific binding to GDF8 under suitable binding conditions, or (2) ability to block the biological activity of GDF8. Typically, polypeptide analogs or variants comprise a conservative amino acid substitution (or insertion or deletion) with respect to the naturally-occurring sequence. Analogs typically are at least 20 amino acids long, at least 50, 60, 70, 80, 90, 100, 150 or 200 amino acids long or longer, and can often be as long as a full-length naturally-occurring polypeptide.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton 1984 W. H. Freeman and Company, New York; Introduction to Protein Structure (Branden & Tooze, eds., 1991, Garland Publishing, NY); and Thornton et at. 1991 Nature 354:105, which are each incorporated herein by reference.

Non-peptide analogs are commonly used in the pharmaceutical industry as drugs with properties analogous to those of the template peptide. These types of non-peptide compound are termed “peptide mimetics” or “peptidomimetics” (see, for example, Fauchere (1986) J. Adv. Drug Res. 15:29; and Evans et al. (1987) J. Med. Chem. 30:1229, which are incorporated herein by reference. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo et al. (1992) Ann. Rev. Biochem. 61:387, incorporated herein by reference), for example, by adding internal cysteine residues capable of forming intramolecular disulfide bridges which cyclize the peptide.

The term “percent sequence identity” in the context of nucleic acid sequences refers to the residues in two sequences which are the same when aligned for maximum correspondence. The length of sequence identity comparison may be over a stretch of at least about nine nucleotides or more, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson (1990) Methods Enzymol. 183:63-98 and (2000) Methods Mol. Biol. 132:185-219, each herein incorporated by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

A reference to a nucleic acid sequence encompasses its complement unless otherwise specified. Thus, a reference to a nucleic acid molecule having a particular sequence should be understood to encompass its complementary strand, with its complementary sequence. Generally, the art uses the terms “percent sequence identity”, “percent sequence similarity” and “percent sequence homology” interchangeably. In this application, these terms shall have the same meaning with respect to nucleic acid sequences.

The term “substantial similarity”, or “substantial sequence similarity,” when referring to a nucleic acid or fragment thereof, indicates that, when optimally aligned with appropriate nucleotide insertions or deletions with another nucleic acid (or its complementary strand), there is nucleotide sequence identity in at least about 90%, at least about 95%, at least about 96%, at least about 97%, at least about 98% or at least about 99% of the nucleotide bases, as measured by any well-known algorithm of sequence identity, such as FASTA, BLAST or Gap, as discussed above.

As applied to polypeptides, the term “substantial identity” or “substantially identical” means that two peptide sequences, when optimally aligned, such as by the programs GAP or BESTFIT using default gap weights, share at least about 80% sequence identity, at least about 90%, at least about 95%, at least about 98% or at least about 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; and 6) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000), supra). Another preferred algorithm when comparing a sequence of the invention to a database containing a large number of sequences from different organisms is the computer program BLAST, especially blastp or tblastn, using default parameters. See, e.g., Altschul et al. (1990) J. Mol. Biol. 215:403-410 and Altschul et al. (1997) Nucleic Acids Res. 25:3389 402, each of which is herein incorporated by reference.

The length of polypeptide sequences compared for homology will generally be at least about 16 amino acid residues, at least about 20 residues, at least about 24 residues, at least about 28 residues, or at least about 35 residues. When searching a database containing sequences from a large number of different organisms, it is preferable to compare amino acid sequences.

The term “effective amount” is a concentration or amount of an antibody or antigen-binding fragment of an antibody which results in achieving a particular stated purpose. An “effective amount” of an anti-GDF8 antibody or antigen-binding fragment of an antibody thereof may be determined empirically. Furthermore, a “therapeutically effective amount” is a concentration or amount of an anti-GDF8 antibody or antigen-binding fragment thereof which is effective for achieving a stated therapeutic effect. This amount may also be determined empirically.

### Preparation of Human Antibodies

Methods for generating monoclonal antibodies, including fully human monoclonal antibodies are known in the art. Any such known methods can be used in the context of the present invention to make human antibodies that specifically bind to GDF8.

Using VELOCIMMUNE™ technology or any other known method for generating monoclonal antibodies, high affinity chimeric antibodies to GDF8 are initially isolated having a human variable region and a mouse constant region. As in the experimental section below, the antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, the antibodies of the instant invention possess very high affinities, typically possessing K_{D} of from about 10⁻¹² through about 10⁻⁹ M, when measured by binding to antigen either immobilized on solid phase or in solution phase. The mouse constant regions are replaced with desired human constant regions to generate the fully human antibodies of the invention, for example wild-type IgG1 (SEQ ID NO:335) or IgG4 (SEQ ID NO:336), or modified IgG1 or IgG4 (for example, SEQ ID NO:337). While the constant region selected may vary according to specific use, high affinity antigen-binding and target specificity characteristics reside in the variable region.

### Bioequivalents

The anti-GDF8 antibodies and antibody fragments of the present invention encompass proteins having amino acid sequences that vary from those of the described antibodies, but that retain the ability to bind human GDF8. Such variant antibodies and antibody fragments comprise one or more additions, deletions, or substitutions of amino acids when compared to parent sequence, but exhibit biological activity that is essentially equivalent to that of the described antibodies. Likewise, the anti-GDF8 antibody-encoding DNA sequences of the present invention encompass sequences that comprise one or more additions, deletions, or substitutions of nucleotides when compared to the disclosed sequence, but that encode an anti-GDF8 antibody or antibody fragment that is essentially bioequivalent to an anti-GDF8 antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-GDF8 antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-GDF8 antibody variants comprising amino acid changes which modify the glycosylation characteristics of the antibodies, e.g., mutations which eliminate or remove glycosylation.

### Epitope Mapping and Related Technologies

To screen for antibodies which bind to a particular epitope (e.g., those which block binding of IgE to its high affinity receptor), a routine cross-blocking assay such as that described in Harlow and Lane (1990) supra can be performed. Other methods include alanine scanning mutants, peptide blots (Reineke (2004) Methods Mol Biol 248:443-63) (herein specifically incorporated by reference in its entirety), or peptide cleavage analysis. In addition, methods such as epitope excision, epitope extraction and chemical modification of antigens can be employed (Tomer (2000) Protein Science 9:487-496) (herein specifically incorporated by reference in its entirety).

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the anti-GDF8 antibodies of the invention into groups of antibodies binding different epitopes.

The invention includes anti-GDF8 antibodies and antigen-binding fragments of antibodies which bind specific epitopes of human GDF8 (SEQ ID NO:340) and are capable of blocking the biological activity of GDF8. In one embodiment, the antibody or antigen-binding fragment thereof binds within an epitope comprising amino acids residues 1 to 109; 1 to 54; 1 to 44; 1 to 34; 1 to 24; and 1 to 14. In another embodiment, the antibody or antigen-binding fragment thereof binds within an epitope comprising of amino acid residues 65 to 72; 35 to 109; 45 to 109; 55 to 109; 65 to 109; 75 to 109; 85 to 109; 92 to 109; or 95 to 109. In another embodiment, the antibody or antigen-binding fragment thereof binds within an epitope comprising amino acid residue 48 to 72; 48 to 69; 48 to 65; 52 to 72; 52 to 65; or 56 to 65. In specific embodiments, the antibody or antigen-binding fragment thereof may bind within 2 or more epitopes.

The present invention also includes antibodies and antigen-binding fragments thereof that bind wild-type mature GDF8 (SEQ ID NO:340) but do not bind isolated peptides having less than the full amino acid sequence of SEQ ID NO:340. For example, the invention includes anti-GDF8 antibodies that bind wild-type mature GDF8 (SEQ ID NO:340) but do not bind isolated peptides consisting of 10 to 40 contiguous amino acids of SEQ ID NO:340. The invention also includes anti-GDF8 antibodies that do not bind any linear epitopes within wild-type mature GDF8. In certain embodiments of the present invention, the anti-GDF8 antibodies bind wild-type mature human GDF8 comprising SEQ ID NO:340 but do not bind one or more isolated GDF8 peptides having an amino acid sequence selected from the group consisting of amino acids 1-14, 1-18, 17-42, 48-65, 48-69, 48-72, 52-65, 52-72, 56-65, 56-72, 65-72, 73-90, 75-105 and 91-105, of SEQ ID NO:340. In certain embodiments, the anti-GDF8 antibodies do not bind any of the aforementioned GDF8 peptides. Methods for determining whether a given antibody is able to bind a particular GDF8 peptide are known to persons of ordinary skill in the art. One exemplary method is illustrated by Example 7 herein, in which GDF8 peptides are attached to microspheres, antibodies are added to the peptide-conjugated microspheres, and, following washing steps, antibody-bound microspheres are detected. The absence of bound antibodies indicates that the antibodies do not bind the particular peptides being tested.

The present invention also includes isolated human antibodies, or antigen-binding fragments thereof, that specifically bind to wild-type mature human GDF8 (e.g., a protein or polypeptide comprising SEQ ID NO:340), but do not bind to a chimeric GDF8 construct in which certain amino acids of GDF8 are replaced with the corresponding amino acid sequence(s) from a non-identical but related protein such as TGFβ-1. In one example, the chimeric construct is a GDF8/TGFβ-1 chimera in which amino acids 48-72 of mature GDF8 are replaced with the corresponding amino acid sequence of TGFβ-1 (e.g., amino acids 49-76 of TGFβ-1). An example of one such chimera is represented by SEQ ID NO:352 (see Examples 4 and 6 herein). Thus, in certain embodiments, the antibodies of the invention specifically bind to wild-type mature human GDF8 (SEQ ID NO:340) but do not bind to the chimeric GDF8/TGFβ-1 construct of SEQ ID NO:352, indicating that the epitope to which such antibodies bind includes or encompasses amino acids located within residues 48 to 72 of SEQ ID NO:340. Blocking bioassays, such as the assay set forth in Example 4 herein, can also be used to indirectly ascertain if an antibody binds wild-type mature human GDF8 (SEQ ID NO:340) and does not bind a chimeric GDF8/TGFβ-1 construct, e.g., the construct of SEQ ID NO:352. For example, an antibody which blocks the bioactivity of wild-type mature human GDF8 but does not block the bioactivity of a chimeric GDF8/TGFβ-1 is deemed to bind to the portion of GDF8 that is replaced by the corresponding TGFβ-1 sequence in the chimeric construct.

Similarly, the present invention also includes isolated human antibodies, or antigen-binding fragments thereof, that block wild-type mature GDF8-mediated activity in a bioassay but do not block activity of a chimeric GDF8 construct (e.g., a GDF8/TGFβ-1 chimera in which amino acids 48-72 of mature GDF8 are replaced with the corresponding amino acid sequence of TGFβ-1 (e.g., SEQ ID NO:352)). An exemplary GDF8 bioassay that can be used in the context of this aspect of the invention is the GDF8-inducible luciferase assay set forth in Example 4 herein, although other similar bioassays capable of measuring the cellular activity of GDF8 are contemplated herein as well.

The present invention includes anti-GDF8 antibodies that bind to the same epitope as any of the specific exemplary antibodies described herein. Likewise, the present invention also includes anti-GDF8 antibodies that cross-compete for binding to GDF8 or a GDF8 fragment with any of the specific exemplary antibodies described herein.

One can easily determine whether an antibody binds to the same epitope as, or competes for binding with, a reference anti-GDF8 antibody by using routine methods known in the art. For example, to determine if a test antibody binds to the same epitope as a reference anti-GDF8 antibody of the invention, the reference antibody is allowed to bind to a GDF8 protein or peptide under saturating conditions. Next, the ability of a test antibody to bind to the GDF8 molecule is assessed. If the test antibody is able to bind to GDF8 following saturation binding with the reference anti-GDF8 antibody, it can be concluded that the test antibody binds to a different epitope than the reference anti-GDF8 antibody. On the other hand, if the test antibody is not able to bind to the GDF8 molecule following saturation binding with the reference anti-GDF8 antibody, then the test antibody may bind to the same epitope as the epitope bound by the reference anti-GDF8 antibody of the invention. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present invention, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990:50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding with a reference anti-GDF8 antibody, the above-described binding methodology is performed in two orientations: In a first orientation, the reference antibody is allowed to bind to a GDF8 molecule under saturating conditions followed by assessment of binding of the test antibody to the GDF8 molecule. In a second orientation, the test antibody is allowed to bind to a GDF8 molecule under saturating conditions followed by assessment of binding of the reference antibody to the GDF8 molecule. If, in both orientations, only the first (saturating) antibody is capable of binding to the GDF8 molecule, then it is concluded that the test antibody and the reference antibody compete for binding to GDF8. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

### Species Selectivity and Species Cross-Reactivity

According to certain embodiments of the invention, the anti-GDF8 antibodies bind to human GDF8 but not to GDF8 from other species. Alternatively, the anti-GDF8 antibodies of the invention, in certain embodiments, bind to human GDF8 and to GDF8 from one or more non-human species. For example, the anti-GDF8 antibodies of the invention may bind to human GDF8 and may bind or not bind, as the case may be, to one or more of mouse, rat, guinea pig, hamster, gerbil, pig, cat, dog, rabbit, goat, sheep, cow, horse, camel, cynomologous, marmoset, rhesus or chimpanzee GDF8.

### Immunoconjugates

The invention encompasses a human or humanized anti-GDF8 monoclonal antibody conjugated to a therapeutic moiety (“immunoconjugate”), such as a cytotoxin, a chemotherapeutic drug, an immunosuppressant or a radioisotope. Cytotoxin agents include any agent that is detrimental to cells. Examples of suitable cytotoxin agents and chemotherapeutic agents for forming immunoconjugates are known in the art, see for example, WO 05/103081, which is herein specifically incorporated by reference).

### Multispecific Antibodies

The antibodies of the present invention may be monospecific, bi-specific, or multispecific. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The anti-GDF8 antibodies of the present invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. For example, the present invention includes bi-specific antibodies wherein one arm of an immunoglobulin is specific for human GDF8 or a fragment thereof, and the other arm of the immunoglobulin is specific for a second therapeutic target or is conjugated to a therapeutic moiety.

An exemplary bi-specific antibody format that can be used in the context of the present invention involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise a Y96F modification (by IMGT; Y436F by EU). Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V4221 by EU) in the case of IgG2 antibodies; and Q15R, N44S, K52N, V57M, R69K, E79Q, and V82I (by IMGT; Q355R, N384S, K392N, V397M, R409K, E419Q, and V4221 by EU) in the case of IgG4 antibodies. Variations on the bi-specific antibody format described above are contemplated within the scope of the present invention.

### Therapeutic Administration and Formulations

The invention provides therapeutic compositions comprising the antibodies or antigen-binding fragments thereof of the present invention. The administration of therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Powell et al. “Compendium of excipients for parenteral formulations” PDA (1998) J Pharm Sci Technol 52:238-311.

The dose may vary depending upon the age and the size of a subject to be administered, target disease, conditions, route of administration, and the like. When the antibody of the present invention is used for treating various conditions and diseases associated with GDF8, for example, muscular dystrophy, muscle atrophy, muscle wasting syndrome, sarcopenia and cachexia, in an adult patient, it is advantageous to intravenously administer the antibody of the present invention normally at a single dose of about 0.01 to about 20 mg/kg body weight, about 0.1 to about 10 mg/kg body weight, or about 0.1 to about 5 mg/kg body weight. Depending on the severity of the condition, the frequency and the duration of the treatment can be adjusted. In other parenteral administration and oral administration, the antibody can be administered in a dose corresponding to the dose given above. When the condition is especially severe, the dose may be increased according to the condition up to the amount that causes significant side effects, if any.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see Langer (1990) Science 249:1527-1533).

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL), to name only a few.

In certain situations, the pharmaceutical composition can be delivered in a controlled release system, for example, with the use of a pump or polymeric materials. In another embodiment, a controlled release system can be placed in proximity of the composition's target, thus requiring only a fraction of the systemic dose.

Examples of the composition for oral administration include solid or liquid dosage forms, specifically, tablets (including dragees and film-coated tablets), pills, granules, powdery preparations, capsules (including soft capsules), syrup, emulsions, suspensions, etc. Such a composition is manufactured by publicly known methods and contains a vehicle, a diluent or an excipient conventionally used in the field of pharmaceutical preparations. Examples of the vehicle or excipient for tablets are lactose, starch, sucrose, magnesium stearate, and the like.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to 500 mg per dosage form in a unit dose; especially in the form of injection, it is preferred that the aforesaid antibody is contained in about 5 to 100 mg and in about 10 to 250 mg for the other dosage forms.

### Therapeutic Uses of the Antibodies

The antibodies of the present invention are useful, inter alia, for the treatment, prevention and/or amelioration of any disease or disorder associated with GDF8 activity. More specifically, the antibodies of the present invention are useful for the treatment of any condition or affliction which can be improved by increasing muscle strength/power and/or muscle mass and/or muscle function in an individual, or by favorably altering metabolism (carbohydrate, lipid and protein processing) by blocking GDF8 activity. Exemplary diseases, disorders and conditions that can be treated with the anti-GDF8 antibodies of the present invention include, but are not limited to, sarcopenia, cachexia (either idiopathic or secondary to other conditions, e.g., cancer, chronic renal failure, or chronic obstructive pulmonary disease), muscle injury, muscle wasting and muscle atrophy, e.g., muscle atrophy or wasting caused by or associated with disuse, immobilization, bed rest, injury, medical treatment or surgical intervention (e.g., hip fracture, hip replacement, knee replacement, etc.) or by necessity of mechanical ventilation. The anti-GDF8 antibodies of the invention may also be used to treat, prevent or ameliorate diseases such as cancer, obesity, diabetes, arthritis, multiple sclerosis, muscular dystrophy, amyotrophic lateral sclerosis, Parkinson's disease, osteoporosis, osteoarthritis, osteopenia, metabolic syndromes (including, but not limited to diabetes, obesity, nutritional disorders, organ atrophy, chronic obstructive pulmonary disease, and anorexia).

The present invention includes therapeutic administration regimens which comprise administering an anti-GDF8 antibody of the present invention in combination with at least one additional therapeutically active component. Non-limiting examples of such additional therapeutically active components include other GDF8 antagonists (e.g., small molecule inhibitors of GDF8 or other GDF8 antibodies or binding molecules), growth factor inhibitors, immunosuppressants, anti-inflammatory agents, metabolic inhibitors, enzyme inhibitors, and cytotoxic/cytostatic agents. The additional therapeutically active component(s) may be administered prior to, concurrent with, or after the administration of the anti-GDF8 antibody of the present invention.

### Diagnostic Uses of the Antibodies

The anti-GDF8 antibodies of the present invention may also be used to detect and/or measure GDF8 in a sample, e.g., for diagnostic purposes. For example, an anti-GDF8 antibody, or fragment thereof, may be used to diagnose a condition or disease characterized by aberrant expression (e.g., over-expression, under-expression, lack of expression, etc.) of GDF8. Exemplary diagnostic assays for GDF8 may comprise, e.g., contacting a sample, obtained from a patient, with an anti-GDF8 antibody of the invention, wherein the anti-GDF8 antibody is labeled with a detectable label or reporter molecule. Alternatively, an unlabeled anti-GDF8 antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure GDF8 in a sample include enzyme-linked immunosorbent assay (ELISA), radioimmunoassay (RIA), and fluorescence-activated cell sorting (FACS).

Samples that can be used in GDF8 diagnostic assays according to the present invention include any tissue or fluid sample obtainable from a patient which contains detectable quantities of GDF8 protein, or fragments thereof, under normal or pathological conditions. Generally, levels of GDF8 in a particular sample obtained from a healthy patient (e.g., a patient not afflicted with a disease or condition associated with abnormal GDF8 levels or activity) will be measured to initially establish a baseline, or standard, level of GDF8. This baseline level of GDF8 can then be compared against the levels of GDF8 measured in samples obtained from individuals suspected of having a GDF8 related disease or condition.

### EXAMPLES

### Example 1. Generation of Human Antibodies to Human GDF8.

Mice may be immunized by any method known in the art (see, for example, Harlow and Lane supra). In one embodiment, GDF8 antigen is administered directly, with an adjuvant to stimulate the immune response, to a VELOCIMMUNE® mouse comprising DNA encoding human Ig heavy and kappa light chain variable regions. Suitable adjuvants include complete and incomplete Freund's adjuvant, MPL+TDM adjuvant system (Sigma), or RIBI (muramyl dipeptides) (see O'Hagan 2000 Vaccine Adjuvant, by Human Press, Totawa, NJ). The antibody immune response is monitored by standard antigen-specific immunoassay. When a desired immune response is achieved, in one embodiment, antibody-expressing B cells are harvested and fused with mouse myeloma cells to preserve their viability and form hybridoma cell lines. The hybridoma cell lines are screened and selected to identify cell lines that produce antigen-specific antibodies.

Alternatively, antigen-specific hybridoma cells may be isolated by flow cytometry. Briefly, after fusion to myeloma cells, pooled hybridoma cells are grown for 10 days in HAT medium. The cells are then harvested and stained with biotin-labeled GDF8 at 2 μg/ml for one hour, followed by addition of phycoerythrin-streptavidin. The fluorescence-labeled cells are sorted by flow cytometry (single cell per well into 96 well plates containing hybridoma growth medium), cultured for 8-10 days, and conditioned media screened for the presence of functionally desirable monoclonal antibodies.

In another embodiment, anti-GDF8 antibodies generated via direct isolation of splenocytes. Antigen-specific antibodies are isolated directly from antigen-immunized B cells without fusion to myeloma cells, as described in U.S. 2007/0280945A1, herein specifically incorporated by reference in its entirety. Stable recombinant antibody-expressing CHO cell lines are established from the isolated proper recombinants.

### Example 2. Gene Utilization Analysis

To analyze the structure of antibodies produced, the nucleic acids encoding antibody variable regions were cloned and sequenced. From the nucleic acid sequence and predicted amino acid sequence of the antibodies, gene usage was identified for each antibody chain. Table 1 sets forth the gene usage for selected antibodies in accordance with the invention. Antibody identifier (HCVR/LCVR): 21-E5 (SEQ ID NO:34/42); 21-B9 (SEQ ID NO:18/26); 21-E9 (SEQ ID NO:98/106); 21-A2 (SEQ ID NO:2/10); 22-D3 (SEQ ID NO:50/58); 22-E6 (SEQ ID NO:66/74); 22-G10 (SEQ ID NO:82/90); 1A2 (SEQ ID NO:226/234); 20B12 (SEQ ID NO:274/282); 58C8 (SEQ ID NO:242/250); 19F2 (SEQ ID NO:258/266); 8D12-1 (SEQ ID NO:114/122); 4E3-7 (SEQ ID NO:194/202); 9B11-12 (SEQ ID NO:162/170); 4B9 (SEQ ID NO:226/234); 1H4-5 (SEQ ID NO:210/218); 9B4-3 (SEQ ID NO:178/186); 3E2-1 (SEQ ID NO:290/298); 4G3-25 (SEQ ID NO:306/314); 4B6-6 (SEQ ID NO:130/138); H4H1657N2 (SEQ ID NO:360/368); H4H1669P (SEQ ID NO:376/384).

TABLE 1

| | |
|---|---|
| Heavy Chain | Light Chain |
| Variable Region | Variable Region |

| Antibody | VH | D | JH | VK | JK |
|---|---|---|---|---|---|
| 21-E5 | 4-39 | 3-22 | 5 | 1-17 | 1 |
| 21-B9 | 4-39 | 3-22 | 5 | 1-17 | 1 |
| 21-E9 | 4-39 | 3-22 | 5 | 1-17 | 1 |
| 21-A2 | 3-23 | 1-7 | 4 | 3-15 | 4 |
| 22-D3 | 3-21 | 5-5 | 4 | 1-17 | 2 |
| 22-E6 | 4-39 | 3-22 | 5 | 1-17 | 1 |
| 22-G10 | 2-5 | 1-7 | 4 | 1-16 | 4 |
| 1A2 | 3-23 | 1-7 | 3 | 3-15 | 4 |
| 20B12 | 3-23 | 6-13 | 6 | 3-15 | 4 |
| 58C8 | 3-23 | 1-7 | 3 | 3-15 | 2 |
| 19F2 | 3-30 | 1-26 | 4 | 2-28 | 3 |
| 8D12-1 | 3-30 | 1-7 | 4 | 2-137* | 5* |
| 4E3-7 | 3-23 | 1-7 | 4 | 3-15 | 4 |
| 9B11-12 | 3-23 | 1-7 | 3 | 3-15 | 4 |
| 4B9 | 3-23 | 1-7 | 3 | 3-15 | 4 |
| 1H4-5 | 3-30 | 6-13 | 6 | 3-15 | 4 |
| 9B4-3 | 3-23 | 1-7 | 4 | 3-15 | 4 |
| 3E2-1 | 4-34 | 4-4 | 4 | 1-9 | 4 |
| 3A4-3 | 3-21 | 5-5 | 4 | 1-17 | 1 |
| 4G3-25 | 3-30 | 3-3 | 4 | 2-28 | 5 |
| 4B6-6 | 3-30 | 1-7 | 4 | 2-137* | 5* |
| H4H1657N2 | 3-23 | 2-21 | 6 | 1-27 | 4 |
| H4H1669P | 3-33 | 3-9 | 6 | 1-12 | 4 |

### Control Constructs Used in the Following Examples

Various control constructs (anti-GDF8 antibodies and other GDF8 antagonists) were included in the following experiments for comparative purposes. The control constructs are designated as follows: Control I: a human anti-GDF8 antibody with heavy and light chain variable regions having the amino acid sequences of the corresponding domains of “Myo29” SEQ ID NOs: 16 and 18) as set forth in U.S. Pat. No. 7,261,893; Control II: a human anti-GDF8 antibody with heavy and light chain variable regions having the amino acid sequences of the corresponding domains of “2_112_K” SEQ ID NOs: 118 and 120) as set forth in US 2006/0263354; Control III: ActRIIB-Fc fusion construct having the amino acid sequence of SEQ ID NO:391; and Control IV: variant ActRIIB-Fc fusion construct, identical to Control III except that alanine at position 64 of SEQ ID NO:391 [A64] is replaced with an arginine [R64]. (Not all control constructs were used in every Example).

### Example 3. Antigen Binding Affinity Determination

Equilibrium dissociation constants (K_{D} values) for antigen binding to selected antibodies were determined by surface kinetics using a real-time biosensor surface plasmon resonance assay (BIACORE™ 2000). Each selected antibody was captured on either a goat anti-mouse IgG polyclonal antibody surface or a goat anti-hFc polyclonal antibody (Jackson Immuno Research Lab) surface created through direct chemical coupling to a BIACORE™ chip to form a captured antibody surface. Human GDF8 homodimer, hGDF11 homodimer, or hGDF5 homodimer at 25 nM was injected over the captured antibody surfaces, and antigen-antibody binding and dissociation were monitored in real time at room temperature. Kinetic analysis was performed to calculate K_{D}, dissociation rate constants (kd), association rate constants (ka) and half-life of antigen/antibody complex dissociation (Table 2).

TABLE 2

| | |
|---|---|
| GDF8 | GDF11 |

| Antibody | K_{D} (nM) | T_{1/2} (min) | K_{D} (nM) | T_{1/2} (min) |
|---|---|---|---|---|
| 21-E5 | 0.26 | 138 | 0.12 | 116 |
| 21-B9 | 0.12 | 126 | 0.064 | 133 |
| 21-E9 | 0.14 | 155 | 0.07 | 221 |
| 21-A2 | 0.40 | 78 | 0.90 | 21 |
| 22-D3 | 1.23 | 34 | 1.09 | 21 |
| 22-E6 | 0.26 | 148 | 0.12 | 87 |
| 22-G10 | 0.250 | 71 | 0.73 | 50 |
| 1A2 | 0.32 | 60 | 0.30 | 28 |
| 20B12 | 0.86 | 39 | 2.08 | 2 |
| 58C8 | 0.62 | 56 | 0.44 | 30 |
| 19F2 | 0.50 | 38 | — | — |
| 8D12-1 | 0.66 | 95 | 1.87 | 23 |
| 4E3-7 | 1.89 | 27 | 1.33 | 29 |
| 9B11-12 | 1.45 | 39 | 1.41 | 29 |
| 4B9 | 0.55 | 55 | 1.09 | 34 |
| 1H4-5 | 0.95 | 54 | 1.48 | 24 |
| 9B4-3 | 1.18 | 50 | 1.08 | 32 |
| 3E2-1 | 2.55 | 45 | 0.70 | 79 |
| 3A4-3 | 1.07 | 137 | 0.51 | 71 |
| 4G3-25 | 3.90 | 25 | 1.41 | 39 |
| 4B6-6 | 0.95 | 121 | 0.55 | 82 |
| Control I | 0.05 | 191 | 0.08 | 136 |
| Control II | 0.3 | 41 | — | — |

The foregoing experiment was also carried out with GDF8 applied over a captured antibody surface of candidate antibodies H4H1669P or H4H1657N2. Preliminary data showed a very slow off rate for both antibodies, suggesting a K_{D} of 1-2 pM or less.

K_{D} values for antigen binding to selected antibodies were also determined as described above with a modified running buffer that does not contain BSA.

TABLE 3

| | |
|---|---|
| GDF8 | GDF11 |

| Antibody | K_{D} (nM) | T_{1/2} (min) | K_{D} (nM) | T_{1/2} (min) |
|---|---|---|---|---|
| 1A2-mlgG | 0.018 | 152 | 0.926 | 1 |
| 1A2-hlgG | 0.006 | 340 | 0.640 | 6 |
| 8D12 | 0.016 | 840 | NB | |
| Control I | 0.002 | 1301 | 0.001 | 105 |
| Control II | 0.071 | 62 | 1.580 | 7 |

Additional antigen binding experiments were conducted in which GDF8 and GDF11 were applied over a surface of selected anti-GDF8 antibodies and control antibodies at 25° C. and 37° C. Equilibrium dissociation constants (K_{D} values) for antigen binding to selected antibodies were determined by surface kinetics using a real-time biosensor surface plasmon resonance assay (BIACORE™ T100). Each selected antibody or control was captured on a goat anti-hFc polyclonal antibody (Jackson Immuno Research Lab Cat# 109-005-098) surface created through direct chemical coupling to a BIACORE™ CM5 sensor chip to form a captured antibody surface. Various concentrations (2.5-0.625 nM, 2-fold dilutions) of hGDF8 homodimer or hGDF11 homodimer (or in some experiments, Activin A) were injected over the captured antibody surfaces, and antigen-antibody binding and dissociation were monitored in real time. Kinetic analysis was performed to calculate K_{D}, dissociation rate constants (kd), association rate constants (ka) and half-life of antigen/antibody complex dissociation. Results are summarized in Table 4. (NB=no binding observed).

TABLE 4

| | Antigen | 25° C. | | 37° C. | |
|---|---|---|---|---|---|
| Inhibitor | Tested | K_{D} (M) | T_{1/2} (min) | K_{D} (M) | T_{1/2} (min) |
| H4H1669P | GDF8 | 3.93E−11 | 131 | 5.84E−11 | 40 |
| | GDF11 | NB | NB | NB | NB |
| H4H1657N2 | GDF8 | 2.83E−11 | 202 | 3.79E−11 | 91 |
| | GDF11 | NB | NB | NB | NB |
| | Activin A | NB | NB | not determined | |
| 1A2-hlgG1 | GDF8 | 6.23E−11 | 96 | 4.62E−11 | 39 |
| | GDF11 | NB | NB | NB | NB |
| Control I | GDF8 | 1.03E−11 | 273 | 2.44E−11 | 70 |
| (Myo29) | GDF11 | 1.46E−11 | 221 | 2.74E−11 | 56 |
| | Activin A | NB | NB | not determined | |
| Control III | GDF8 | 3.07E−11 | 111 | 6.40E−12 | 104 |
| (ActRIIB- | GDF11 | 2.38E−11 | 132 | 9.92E−12 | 53 |
| hFc [A64]) | Activin A | 8.50E−12 | 196 | not determined | |
| Control IV | GDF8 | 2.13E−11 | 238 | not determined | |
| (ActRIIB- | GDF11 | 3.00E−12 | 231 | | |
| hFc [R64]) | Activin A | 3.00E−12 | 439 | | |
| Isotype | GDF8 | NB | NB | NB | NB |
| negative | GDF11 | NB | NB | NB | NB |

| | | | | |
|---|---|---|---|---|
| control | Activin A | NB | NB | not determined |
| antibody | | | | |

As shown above, antibodies H4H1669P, H4H1657N2, and 1A2-hIgG1 of the present invention all exhibited strong binding to GDF8 but no binding to GDF11. By contrast, the control molecules showed binding to both GDF8 and GDF11.

### Example 4. Antibody Blocking of Smad2/Luciferase Response

GDF8-Inducible Luciferase Assay. A bioassay was developed to determine the ability of selected anti-GDF8 antibodies to neutralize GDF8-mediated or GDF11-mediated cellular function in vitro using an engineered A204 cell line (human rhabdomyosarcoma cells, ATCC) that contains a GDF8 or GDF11-responsive promoter driving luciferase expression. Inhibition of GDF8 or GDF11-inducible luciferase activity was determined as follows: Cells were seeded onto 96-well plate at 2×10⁴ cells/well in media and incubated overnight at 37° C., 5% CO₂. Antibody protein (in serial dilutions starting from 25 nM in cell media) was added to the wells of A204/Smad2 cells in triplicate on two plates; GDF8 or GDF11 (0.8 nM) was added to each well. The plates were incubated at 37° C., 5% CO₂ for 6 hours. Luciferase activity was determined by adding BRIGHT-GLO® Substrate (Promega) and IC₅₀ values determined (Table 5).

TABLE 5

IC₅₀ (nM)

| Antibody | GDF8 | GDF11 |
|---|---|---|
| 21-E5 | 8.50 | 35 |
| 21-B9 | 0.62 | 1.2 |
| 21-E9 | 0.99 | 1.2 |
| 21-A2 | 9.70 | >10 |
| 22-D3 | >20 | >25 |
| 22-E6 | 2.20 | 22.4 |
| 22-G10 | 10.50 | >25 |
| 1A2 | 0.80 | 1400 |
| 20B12 | >20 | >25 |
| 58C8 | 1.80 | >25 |
| 19F2 | >20 | >25 |
| 8D12-1 | 2.40 | >25 |
| 4E3-7 | 10.40 | >25 |
| 9B11-12 | 5.50 | >1000 |
| 4B9 | 0.47 | >25 |
| 3A4-3 | 3.10 | >20 |
| 4G3-25 | >25 | >25 |
| Control I | 0.62 | 0.94 |

The ability of selected anti-GDF8 antibodies to neutralize GDF8-mediated or GDF11-mediated cellular function was further analyzed as described above with varied concentrations of GDF8 or GDF11. (Table 6). (nd=not determined; NB=no binding).

TABLE 6

| IC₅₀ (nM) | |
|---|---|
| GDF8 | GDF11 |

| Antibody | 0.5 nM | 1 nM | 0.4 nM | 0.8 nM |
|---|---|---|---|---|
| 1A2 | 0.196 | 0.363 | ~600 | ~800 |
| 8D12 | 3.11 | 5.55 | >1000 | >1000 |
| 21-E5 | 2.34 | 2.98 | 6.9 | 10.5 |
| H4H1657N2 | 0.78 | nd | NB | nd |
| H4H1669P | 0.90 | nd | NB | nd |
| Control I | 0.172 | 0.398 | 0.255 | 0.459 |
| Control II | 1.6 | 4.15 | >1000 | >1000 |

The bioassay described above was repeated using a GDF8/TGFβ-1 chimeric construct as the activating peptide. In particular, a chimera consisting of mature GDF8 with amino acids 48-72 replaced with the corresponding amino acids of TGFβ-1 was used in this experiment (SEQ ID NO:352, also referred to herein as “GDF8/TGFβ[48-72]”). This was produced from an expression construct encoding the entire human GDF8 precursor with the human TGFβ-1 sequence replacing the corresponding GDF8 sequence. Bioactivity was assayed in conditioned medium produced by transient transfection of the GDF8/TGFβ[48-72] construct in CHO cells. Expression and processing were assessed by Western blot. The conditioned medium was concentrated 20-fold, heated to 80° C. for 5 minutes to inactivate the bound GDF8 propeptide, and assessed for activity in serial dilution in the bioassay. While the precise concentration of chimeric protein was not determined in these experiments, the typical concentration was in the range of 1-10 ug/ml prior to concentration. As described above, cells containing a GDF8-responsive promoter driving luciferase expression were seeded onto a 96-well plate. Selected antibodies (in serial dilutions starting at 100 nM in cell media) were added to an amount of the GDF8/TGFβ[48-72] chimeric protein conditioned medium determined to give maximal response. This mixture was preincubated for 45 minutes and added to the reporter cells. Luciferase activity was measured and approximate IC₅₀ values were calculated, as shown in Table 7 below. (NB=no blocking).

TABLE 7
Antibody Blocking of GDF8/TGFβ[48-72] Bioactivity

| Antibody | IC₅₀ (nM) |
|---|---|
| 1A2-hFc | 0.357 |
| 8D12-mFc | NB |
| H2M1657N2 | NB |
| H1H1669P | NB |
| Control I | 0.402 |

The GDF8/TGFβ[48-72] chimeric construct was able to activate luciferase expression in this assay, and 1A2-hFc and Control I were able to block bioactivity of this construct. However, antibodies H2M1657N2 and H1H1669P failed to block the bioactivity of the GDF8/TGFβ[48-72] chimeric construct. Since these two antibodies were shown to block the bioactivity of wild-type GDF8 in this assay system (see Table 6), it can be concluded that H2M1657N2 and H1H1669P most likely exert their biological effects by interacting with an epitope within amino acids 48-72 of GDF8.

### Example 5. Immunoblotting hGDF8 Fragments Generated by Proteinase K Digestion

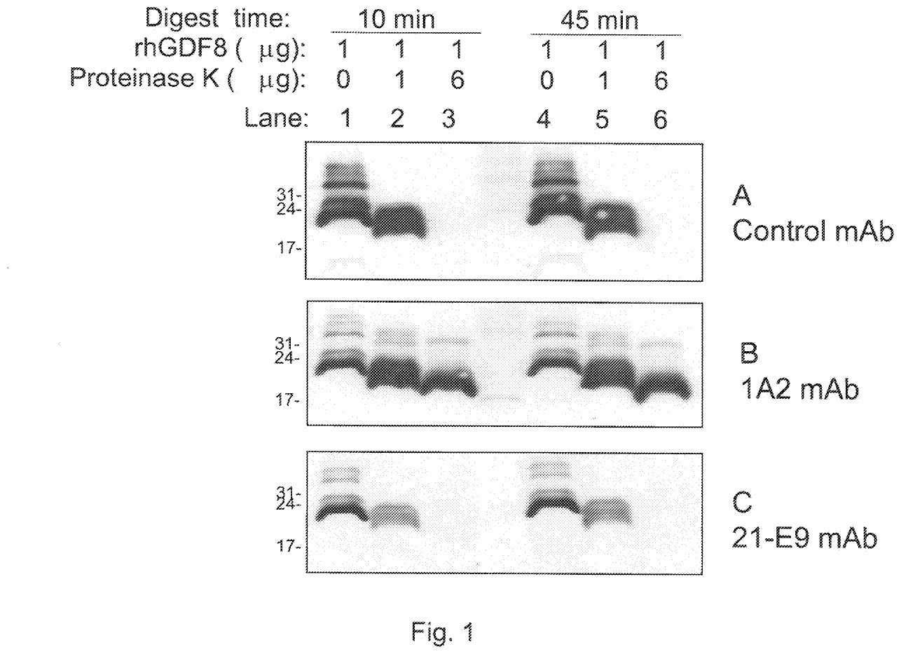

Western blot analysis was used to determine the immunoreactivity of test and control mAbs human GDF8 proteolytically digested with Proteinase K. Enzyme reactions containing 1 μg human GDF8 and 0, 1 or 6 μg Proteinase K were incubated for either 10 or 45-min in digestion buffer. Equal aliquots containing 20% of the amount of GDF8 present in the reaction mixture (200 ng) was loaded into 3 separate 18% SDS-PAGE non-reducing gels and electroblotted to PVDF membranes. Each membrane was incubated with primary antibody at 2 μg/ml followed by the appropriate secondary antibody conjugated to HRP. Shown in FIG. 1A-C, is the resulting immunoreactivity detected for the control mAb I, 1A2 mAb and 21-E9 mAb, respectively. Results show a loss of GDF8 reactivity for the control mAb (A) in lanes 3 and 6. In marked contrast, antibody 1A2 retains immunoreactivity to a smaller GDF8 fragment of approximately 17-19 kD in molecular weight (FIG. 1B).

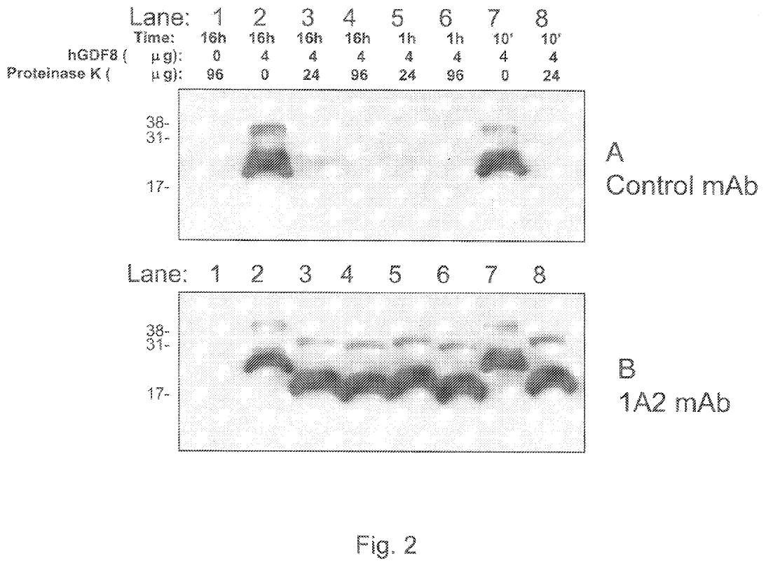

The experiment was repeated with digestion times of up 10 min, 1 hr or 16 hr, in the presence of 0 or 4 μg of GDF8, and 0, 24 or 96 μg Proteinase K. The results (FIG. 2A-B) show that in the absence of Proteinase K, both control and 1A2 mAbs were immunoreactive with full length mature (undigested) GDF8 (see FIG. 2A-B, Lanes 2 and 7). In the presence of Proteinase K, immunoreactivity is lost for the control I mAb at all time points. In marked contrast, 1A2 mAb remained immunoreactive with digested human GDF8 fragment (FIG. 2B, Lanes 3-6 and 8). These results indicate that 1A2 mAb is immunoreactive with a smaller fragment of GDF8 that remains intact in the presence of Proteinase K, whereas control mAb loses immunoreactivity to the smaller fragment (17-19 kD).

A modified Western blot analysis was used to further determine the hGDF8 epitope for selected anti-hGDF8 antibodies. The modification being that before the hGDF8 specific primary antibody was incubated with the membrane, each anti-hGDF8 antibody was pre-incubated with 1000 fold or 50 fold molar excess of hGDF8 peptide fragments of 1-14 amino acids, 17-42 amino acids, 48-72 amino acids, or 75-105 amino acids of SEQ ID NO: 340. The results show that pre-incubation of peptide fragment 48-72 amino acids of SEQ ID NO: 340 at 50 fold molar excess was able to block the binding of antibody 8D12 to hGDF8.

### Example 6. Antibody Binding to GDF8 Chimeras

Twelve chimeric GDF8 pro-proteins were made. Table 8 shows the mature chimeric GDF8 protein structures. The chimeric GDF8 proteins comprised two sets: one set having various substitutions of GDF8 sequences with BMP2 sequences, the other set having various substitutions of GDF8 sequences with TGFβ1 sequences. These chimeric proteins were used to test and localize antibody binding.

TABLE 8

| | Substituted | | |
|---|---|---|---|
| | Mature | | SEQ |
| Chimera | GDF8 | | ID |
| Name | Fragment | Mature Chimeric GDF8 Structure | NO: |
| B1 | 1-15 | BMP2.1-13.GDF8.16-109 | 347 |
| T1 | 1-15 | TGFb1.1-15.GDF8.16-109 | 348 |
| B17 | 17-42 | GDF8.1-16.BMP2.15-42.GDF8.43-109 | 349 |
| T17 | 17-42 | GDF8.1-16, TGFβ1.17-43.GDF8.43- | 350 |
| | | 109 | |
| B48 | 48-72 | GDF8.1-47.BMP2.48-77.GDF8.73-109 | 351 |
| T48 | 48-72 | GDF8.1-47.TGFβ1.49-76.GDF8.73- | 352 |
| | | 109 | |
| B65 | 65-72 | GDF8.1-64.BMP2.68-77GDF8.73-109 | 353 |
| T65 | 65-72 | GDF8.1-64.TGFβ1.69-76.GDF8.73- | 354 |
| | | 109 | |
| B75 | 75-105 | GDF8.1-74.BMP2.80-110DGF8.106- | 355 |
| | | 109 | |
| T75 | 75-105 | GDF8.1-74.TGFβ1.79-108.GDF8.106- | 356 |
| | | 109 | |
| B91 | 91-105 | GDF8.1-90.BMP2.96-110.GDF8.106- | 357 |
| | | 109 | |
| T91 | 91-105 | GDF8.1-90.TGFβ1.95-10GDF8.106- | 358 |
| | | 109 | |

The various chimeric GDF8 pro-proteins were transiently transfected in an engineered stable CHO.hFurin cell line. A similar Western Blot analysis as described above was used to detect the binding of various anti-hGDF8 antibodies to each of the chimeric GDF8. Briefly, 10 μg of CHO supernatant were loaded onto each lane of an SDS-PAGE (non-reducing or reducing) gel and electroblotted onto a PVDF membrane. The membrane was then incubated with an anti-GDF8 antibody at 2 μg/ml followed by exposure to the appropriate secondary antibody conjugated to HRP. As shown in Table 9, antibody 8D12 was not able to bind either B48 or T48. The result indicated that amino acids 48 to 72 of mature GDF8 participate in the binding of antibody 8D12 to GDF8.

TABLE 9

| | Non Reducing | | | | Reducing | |
|---|---|---|---|---|---|---|
| GDF8 Protein | Control I | 8D12 | Control II | 1A2 | 4A7 | 8D2 |
| Wild Type GDF8 | + | + | + | + | + | + |
| B48 | + | − | + | + | + | − |
| B65 | + | + | + | + | + | + |
| B91 | + | + | + | + | + | + |
| T48 | + | − | + | + | + | − |
| T65 | + | + | + | + | + | + |

### Example 7. Antibody Binding to hGDF8 Peptides

Fourteen peptides (Table 10) were generated from mature hGDF8 (SEQ ID NO:340). Unmodified peptides, N-terminal biotinylated peptides (N-term), or C-terminal biotinylated peptides (C-term) were used to test and localize antibody binding. Full-length hGDF8, hGDF11 and unmodified peptides were each individually amine-coupled to xMAP® Multi-Analyte COOH Microspheres (or beads). Each of the biotinylated peptides was bound to xMAP® Multi-Analyte LumAvidin Microspheres. Peptide-bound beads suspension were then mixed with an equal volume of blocking buffer (PBS, 1% BSA, 0.05% Tween20, 0.05% Sodium azide) and then distributed into a 96 well filter plate (Millipore, MULTISCREEN® BV). Control and test anti-hGDF8 antibodies, at 2.5 μg/ml were then added to the peptide-bound beads suspension and were allowed to bind to the beads at RT, overnight. The antibody-incubated beads were then washed twice with PBST (PBS+0.05% Tween20) and incubated with either Phycoerythrin (PE) conjugated anti-hFC or PE-conjugated anti-mFC antibodies at RT for 45 min. The beads were washed again and the antibody binding signal to various peptides were detected with either LUMINEX® 100™ or 200™ instruments. As shown in Table 10, anti-hGDF8 antibody 8D12 is able to bind to peptides 4, 5, 6, 7, 8, and 9. By contrast, anti-hGDF8 antibody H4H1657N2 did not bind any of the peptides (data not shown).

TABLE 10

| | Pep- | Modifi- | Con- | Con- | | | |
|---|---|---|---|---|---|---|---|
| No. | tide | cation | trol I | trol II | 21-E5 | 1A2 | 8D12 |
| 1 | 1-14 | Unmodified | − | − | − | − | − |
| | | N-term | − | − | − | − | − |
| | | C-term | − | − | − | − | − |
| 2 | 1-18 | Unmodified | − | − | − | − | − |
| | | N-term | − | − | − | − | − |
| | | C-term | − | ++ | − | +++ | +/− |
| 3 | 17-42 | Unmodified | − | − | − | − | − |
| | | N-term | − | − | − | − | − |
| | | C-term | − | − | − | − | − |
| 4 | 48-65 | Unmodified | − | − | − | − | +++ |
| | | C-term | − | − | − | − | +++++ |
| 5 | 48-69 | Unmodified | − | − | − | − | +++ |
| | | C-term | − | − | − | − | +++++ |
| 6 | 48-72 | Unmodified | − | + | + | + | +++ |
| | | N-term | − | − | − | − | + |
| | | C-term | − | − | − | − | +++++ |
| 7 | 52-65 | Unmodified | − | − | − | − | +++ |
| | | C-term | − | − | − | − | +++++ |
| 8 | 52-72 | Unmodified | − | − | − | − | ++ |
| | | C-term | − | − | − | − | +++++ |
| 9 | 56-65 | C-term | − | − | − | − | ++ |
| 10 | 56-72 | Unmodified | − | − | − | − | − |
| | | C-term | − | − | − | − | + |
| 11 | 65-72 | Unmodified | − | − | − | − | − |
| | | N-term | − | − | − | − | − |
| | | C-term | − | − | − | − | − |
| 12 | 73-90 | Unmodified | − | − | − | − | +/− |
| | | N-term | − | +/− | − | + | +/− |
| | | C-term | + | − | − | − | + |
| 13 | 75-105 | Unmodified | − | − | − | − | − |
| | | N-term | − | − | − | − | − |
| | | C-term | − | − | − | − | − |
| 14 | 91-105 | Unmodified | − | − | − | − | − |
| | | N-term | − | − | − | − | − |
| | | C-term | − | − | − | − | − |

### Example 8. Effect of Human Anti-GDF8 Antibodies on the Binding GDF8 to Activin RIIB

Mature hGDF8 was first amine-coupled to Luminex® beads. The hGDF8-coated Luminex® beads were then incubated with various anti-hGDF8 antibodies, at 1.25 μg/ml, for 2 hr at room temperature. Human Activin RIIB-mFc was then added to the bead-antibody mixture and incubated for an additional 2 hr at room temperature. The beads were then washed and stained with R-phycoerythrin (R-PE)-conjugated anti-mFc polyclonal antibody and mean fluorescence intensity (MFI) was measured. As shown in Table 11, although Control mAb I and antibody 21-E5 were both able to block the binding between hGDF8 and hActivin RIIB, Control mAb 11 was only able to partially block binding. Antibody 1A2 was not able to block the binding of hGDF8 to its receptor, Activin RIIB (Table 11, n=3).

TABLE 11

| Antibody | MFI |
|---|---|
| Negative Control | 4,560 |
| Control mAb I | 58 |
| Control mAb II | 1,653 |
| 1A2-hlgG | 4,037 |
| 21-E5-hlgG | 275 |

### Example 9. Antibody 8D12 Variants

Antibody 8D12 variants with modified LCVR were generated by modifying one or more of the following amino acids of the LCVR of 8D12: A7S or T, A8P, P9L, S18P, V19A, M21I, K27Q, F41Y, V42L, R44K, R55L or T, M56G or L, N58Y, L59R, A75D, R79K, A105G, L109V, and L111I.

The binding affinity (K_{D}) of the antibody variants with respect to hGDF8 was determined using a real-time biosensor surface plasma resonance assay (BIAcore™3000) described above in modified running buffer that does not contain BSA (Table 12).

TABLE 12

| Antibody | K_{D} (nM) | T_{1/2} (min) |
|---|---|---|
| 8D12 | 0.071 | 139 |
| 8D12-v2 | 0.520 | 25 |
| 8D12-v3 | 0.380 | 46 |

The binding between antibody variants and hGDF8 peptides was also tested as described above in Example 7. Antibody variants 8D12-v2 and 8D12-v3 showed strong binding to C-terminal biotinylated peptides 4, 5, 7, and 10.

### Example 10. Effect of Anti-GDF8 Antibodies on Skeletal Muscle Mass

The efficacy of selected anti-GDF8 mAbs for inducing skeletal muscle hypertrophy was determined in vivo. Briefly, 20 male CB17 SCID mice, approximately 9 weeks old, were divided evenly according to body weight into 4 groups. A selected mAb (Control I, 1A2, 21-E5, 8D12, 1A2-hIgG, or Control II) was injected at three increasing doses of 2.5 mg/kg/dose, 5 mg/kg/dose, and 10 mg/kg/dose. The Fc fragment of human IgG was used as negative control. Antibodies were administered intraperitoneally twice for the first week and once a week for the following three weeks. On day 28, mice were euthanized and weighed, and the tibialis anterior (TA) muscles, the gastrocnemius (GA) muscles, and quadriceps (Quad) muscles, heart, spleen, and kidney, were dissected and weighed. Tissues were normalized to starting body weight, and percent change in weight over the negative control was calculated. Six separate experiments were repeated with antibodies: Control I, 1A2, 21-E5, 8D12, 1A2-hIgG, and Control II (Table 13-18). In addition, the experiment was also repeated with higher increasing doses of control I antibody at 10 mg/kg/dose, 30 mg/kg/dose, and 50 mg/kg/dose (Table 19). Results are expressed as percent increase over negative control±standard deviation.

TABLE 13

| | | | | Negative |
|---|---|---|---|---|
| | Control I | | | Control |
| Dose | 2.5 mg/kg | 5 mg/kg | 10 mg/kg | 10 mg/kg |
| Body Weight | 7.66 ± 1.37 | 9.92 ± 1.82 | 13.99 ± 1.21 | 0.00 ± 1.41 |
| TA Muscle | 13.84 ± 2.23 | 16.99 ± 2.16 | 13.68 ± 0.96 | 0.00 ± 2.71 |
| GA Muscle | 10.58 ± 1.67 | 11.31 ± 2.44 | 12.90 ± 3.0 | 0.00 ± 3.36 |
| Quad Muscle | 14.79 ± 1.55 | 15.99 ± 2.72 | 20.84 ± 2.09 | 0.00 ± 1.84 |
| Heart | 2.05 ± 3.04 | 2.96 ± 2.96 | 7.55 ± 1.61 | 0.00 ± 2.45 |
| Kidney | 3.53 ± 1.39 | 3.56 ± 3.51 | 5.66 ± 4.59 | 0.00 ± 2.82 |
| Spleen | 39.82 ± 6.78 | 45.26 ± 19.10 | 9.02 ± 3.08 | 0.00 ± 5.47 |

TABLE 14

| | | | | Negative |
|---|---|---|---|---|
| | 1A2 | | | Control |
| Dose | 2.5 mg/kg | 5 mg/kg | 10 mg/kg | 10 mg/kg |
| Body Weight | 6.08 ± 1.08 | 9.96 ± 1.32 | 9.92 ± 1.09 | 0.00 ± 1.76 |
| TA Muscle | 15.56 ± 1.54 | 18.24 ± 4.49 | 20.69 ± 3.13 | 0.00 ± 4.47 |
| GA Muscle | 20.49 ± 1.84 | 21.36 ± 2.79 | 24.36 ± 3.46 | 0.00 ± 3.59 |
| Quad Muscle | 26.92 ± 3.07 | 30.15 ± 3.56 | 33.09 ± 4.46 | 0.00 ± 4.05 |
| Heart | 3.70 ± 1.31 | 6.37 ± 2.27 | 12.42 ± 2.70 | 0.00 ± 4.10 |
| Kidney | 1.28 ± 2.89 | 2.89 ± 3.30 | 5.31 ± 3.29 | 0.00 ± 4.39 |
| Spleen | −8.07 ± 5.75 | −10.00 ± 4.68 | 9.68 ± 9.19 | 0.00 ± 6.84 |

TABLE 15

| | 21-E5 | | | Negative Control |
|---|---|---|---|---|
| Dose | 2.5 mg/kg | 5 mg/kg | 10 mg/kg | 10 mg/kg |
| Body Weight | 4.16 ± 0.87 | 4.14 ± 2.82 | 5.21 ± 1.58 | 0.00 ± 1.15 |
| TA Muscle | 13.86 ± 1.66 | 14.01 ± 2.41 | 10.32 ± 2.54 | 0.00 ± 1.65 |
| GA Muscle | 7.70 ± 1.86 | 12.94 ± 1.10 | 8.13 ± 2.36 | 0.00 ± 1.41 |
| Quad Muscle | 8.64 ± 1.31 | 13.57 ± 1.79 | 8.46 ± 3.22 | 0.00 ± 1.94 |
| Heart | −7.11 ± 1.00 | −7.14 ± 3.17 | −5.51 ± 1.58 | 0.00 ± 2.23 |
| Kidney | −6.8 ± 2.83 | −3.2 ± 3.57 | −0.32 ± 2.07 | 0.00 ± 3.93 |
| Spleen | 29.81 ± 9.83 | 49.76 ± 7.86 | 10.85 ± 6.63 | 0.00 ± 5.76 |

TABLE 16

| | | | | Negative |
|---|---|---|---|---|
| | 8D12 Antibody | | | Control |
| Dose | 2.5 mg/kg | 5 mg/kg | 10 mg/kg | 10 mg/kg |
| Body Weight | 10.06 ± 0.86 | 12.76 ± 1.01 | 11.41 ± 1.35 | 0.00 ± 0.66 |
| TA Muscle | 17.99 ± 1.53 | 21.30 ± 2.06 | 22.11 ± 3.20 | 0.00 ± 1.85 |
| GA Muscle | 21.14 ± 1.19 | 23.10 ± 0.99 | 23.40 ± 3.72 | 0.00 ± 1.44 |
| Quad Muscle | 26.74 ± 1.53 | 31.00 ± 1.61 | 28.80 ± 2.72 | 0.00 ± 0.94 |
| Heart | −1.61 ± 2.06 | 3.63 ± 1.93 | 3.42 ± 2.52 | 0.00 ± 1.08 |
| Kidney | −1.06 ± 2.02 | −4.26 ± 2.25 | −5.52 ± 3.83 | 0.00 ± 3.66 |
| Spleen | 4.33 ± 6.34 | −9.04 ± 2.64 | −1.85 ± 6.26 | 0.00 ± 4.25 |

TABLE 17

| | 1A2-hIgG Antibody | | | Negative Control |
|---|---|---|---|---|
| Dose | 2.5 mg/kg | 5 mg/kg | 10 mg/kg | 10 mg/kg |
| Body Weight | 6.73 ± 1.51 | 3.84 ± 2.32 | 7.45 ± 2.91 | 0.00 ± 0.76 |
| TA Muscle | 15.62 ± 2.4 | 12.09 ± 1.81 | 10.91± 3.33 | 0.00 ± 2.80 |
| GA Muscle | 16.29 ± 1.02 | 15.20 ± 2.54 | 15.67 ± 3.60 | 0.00 ± 2.13 |
| Quad Muscle | 19.39 ± 2.92 | 20.03 ± 2.54 | 19.73 ± 3.83 | 0.00 ± 1.98 |
| Heart | 5.91 ± 3.81 | 5.39 ± 2.77 | 3.52 ± 3.16 | 0.00 ± 3.14 |
| Kidney | 1.70 ± 4.01 | 1.20 ± 1.73 | 1.39 ± 3.70 | 0.00 ± 3.27 |
| Spleen | −15.44 ± 5.6 | −30.08 ± 6.63 | −23.90 ± 2.36 | 0.00 ± 11.32 |

TABLE 18

| | | | Negative |
|---|---|---|---|
| | Control II | | Control |
| Dose | 2.5 mg/kg | 5 mg/kg | 5 mg/kg |
| Body Weight | 10.19 ± 1.20 | 10.83 ± 1.58 | 0.00 ± 1.17 |
| TA Muscle | 14.70 ± 3.28 | 13.44 ± 4.13 | 0.00 ± 3.87 |
| GA Muscle | 19.44 ± 2.08 | 14.35 ± 3.36 | 0.00 ± 1.80 |
| Quad Muscle | 18.92 ± 4.86 | 13.00 ± 2.18 | 0.00 ± 2.03 |
| Heart | 2.78 ± 5.09 | 2.71 ± 6.72 | 0.00 ± 0.69 |
| Kidney | 1.84 ± 2.65 | 0.41 ± 3.04 | 0.00 ± 2.12 |
| Spleen | 12.32 ± 5.60 | 8.41 ± 3.38 | 0.00 ± 4.31 |

TABLE 19

| | | | | Negative |
|---|---|---|---|---|
| | Control I | | | Control |
| Dose | 10 mg/kg | 30 mg/kg | 50 mg/kg | 10 mg/kg |
| Body Weight | 9.20 ± 0.73 | 13.62 ± 1.38 | 12.55 ± 1.71 | 0.00 ± 1.18 |
| TA Muscle | 18.16 ± 1.51 | 25.58 ± 3.14 | 23.03 ± 2.00 | 0.00 ± 3.81 |
| GA Muscle | 16.91 ± 2.08 | 23.72 ± 3.14 | 24.68 ± 2.73 | 0.00 ± 3.58 |
| Quad Muscle | 20.77 ± 1.01 | 26.54 ± 3.68 | 25.67 ± 3.32 | 0.00 ± 4.63 |
| Heart | 1.55 ± 3.07 | 3.11 ± 2.58 | 2.11 ± 2.23 | 0.00 ± 1.96 |
| Kidney | 3.09 ± 1.90 | 10.38 ± 5.08 | 8.89 ± 3.59 | 0.00 ± 1.35 |
| Spleen | 8.22 ± 5.80 | 6.90 ± 5.87 | −0.74 ± 1.85 | 0.00 ± 7.38 |

A similar experiment was carried out using antibodies H4H1657N2 and H4H1669P and controls administered to SCID mice. In particular, male SCID mice at 10 weeks of age were administered antibody subcutaneously at 10 mg/kg according to the following dosing schedule: 2× on week 1 and 1×/week on weeks 2 and 3. The total treatment time was 28 days. For this experiment, 5 mice were administered an isotype negative control antibody; 5 mice were administered Control I (Myo29); 6 mice were administered H4H1657N2; and 6 mice were administered H4H1669P. Results are summarized in Table 20 and are expressed as percent increase over negative control±standard deviation.

TABLE 20

| | Isotype | | | |
|---|---|---|---|---|
| | Control | Control I | H4H1657N2 | H4H1669P |
| Dose | 10 mg/kg | 10 mg/kg | 10 mg/kg | 10 mg/kg |
| Body Weight | 0.00 ± 1.97 | 14.52 ± 2.68 | 10.28 ± 0.95 | 10.70 ± 1.26 |
| TA Muscle | 0.00 ± 4.10 | 17.47 ± 3.09 | 25.53 ± 3.96 | 13.77 ± 2.01 |
| GA Muscle | 0.00 ± 1.97 | 19.46 ± 2.92 | 21.69 ± 1.67 | 13.39 ± 1.30 |
| Quad Muscle | 0.00 ± 3.59 | 14.05 ± 3.03 | 22.15 ± 3.47 | 9.87 ± 2.55 |
| Heart | 0.00 ± 2.49 | 9.42 ± 1.63 | 5.40 ± 2.28 | 12.70 ± 2.67 |
| White Adipose | 0.00 ± 9.72 | 25.14 ± 19 | −8.05 ± 7.53 | 4.22 ± 6.19 |
| Tissue | | | | |

A similar experiment was also carried out using antibodies H4H1657N2 and H4H1669P and controls administered to C57 mice. In particular, male C57 mice at 10 weeks of age were administered antibody subcutaneously at 10 mg/kg according to the following dosing schedule: 2× per week for two weeks. For this experiment, 5 mice were administered an isotype negative control antibody; 5 mice were administered Control I (Myo29); 5 mice were administered H4H1657N2; and 6 mice were administered H4H1669P. Results are summarized in Table 21 and are expressed as percent increase over negative control±standard deviation.

TABLE 21

| | Isotype | | | |
|---|---|---|---|---|
| | Control | Control I | H4H1657N2 | H4H1669P |
| Dose | 10 mg/kg | 10 mg/kg | 10 mg/kg | 10 mg/kg |
| Body Weight | 0.00 ± 0.92 | 4.64 ± 0.99 | 5.11 ± 0.73 | 2.75 ± 1.07 |
| TA Muscle | 0.00 ± 4.26 | 8.92 ± 1.56 | 14.92 ± 3.09 | 7.62 ± 2.90 |
| GA Muscle | 0.00 ± 2.39 | 6.28 ± 2.10 | 14.20 ± 1.77 | 6.07 ± 3.80 |
| Quad Muscle | 0.00 ± 2.46 | 3.53 ± 2.11 | 12.85 ± 2.69 | 1.24 ± 1.74 |
| Heart | 0.00 ± 2.28 | −3.18 ± 2.70 | 1.39 ± 2.83 | 2.45 ± 3.95 |
| White Adipose Tissue | 0.00 ± 8.20 | 19.05 ± 7.72 | −5.23 ± 8.34 | −4.53 ± 10.28 |

Next, dose response experiments were carried out using antibodies H4H1657N2 and H4H1669P in SCID mice. In particular, male SCID mice at 10 weeks of age were administered control antibodies subcutaneously at 30 mg/kg, and H4H1657N2 or H4H1669P at 2.5, 10 or 30 mg/kg, according to the following dosing schedule: 2×per week on week 1 and 1×per week thereafter. Results are summarized in Tables 22 and 23 and are expressed as percent increase over negative control±standard deviation.

TABLE 22

| | Isotype | | | | |
|---|---|---|---|---|---|
| | Control | Control I | H4H1657N2 | | |
| Dose | 30 mg/kg | 30 mg/kg | 2.5 mg/kg | 10 mg/kg | 30 mg/kg |
| Number of Mice (n) | 5 | 5 | 5 | 5 | 5 |
| Body Weight | 0.00 ± 1.38 | 7.46 ± 2.44 | 10.16 ± 0.92 | 6.51 ± 0.91 | 10.12 ± 1.68 |
| TA Muscle | 0.00 ± 2.55 | 16.13 ± 5.62 | 22.22 ± 2.75 | 19.79 ± 1.19 | 24.10 ± 3.62 |
| GA Muscle | 0.00 ± 2.66 | 13.50 ± 5.20 | 21.94 ± 3.18 | 21.84 ± 2.94 | 25.01 ± 4.15 |
| Quad Muscle | 0.00 ± 2.06 | 16.89 ± 4.11 | 26.30 ± 3.72 | 26.28 ± 2.39 | 30.81 ± 2.98 |
| Heart | 0.00 ± 2.67 | 3.23 ± 3.29 | 14.46 ± 1.55 | 2.70 ± 1.51 | 4.06 ± 2.53 |
| White Adipose Tissue | 0.00 ± 13.91 | 5.08 ± 9.02 | −23.70 ± 10.29 | −16.77 ± 7.54 | −15.44 ± 11.43 |

TABLE 23

| | Isotype | | | | |
|---|---|---|---|---|---|
| | Control | Control I | H4H1669P | | |
| Dose | 30 mg/kg | 30 mg/kg | 2.5 mg/kg | 10 mg/kg | 30 mg/kg |
| Number of Mice (n) | 5 | 5 | 5 | 5 | 5 |
| Body Weight | 0.00 ± 1.47 | 13.77 ± 1.99 | 2.87 ± 1.66 | 5.72 ± 0.56 | 4.88 ± 1.11 |
| TA Muscle | 0.00 ± 2.38 | 25.79 ± 2.37 | 7.82 ± 2.25 | 12.38 ± 2.56 | 11.86 ± 2.59 |
| GA Muscle | 0.00 ± 2.12 | 22.82 ± 1.25 | 2.68 ± 2.06 | 12.46 ± 1.30 | 10.55 ± 3.33 |
| Quad Muscle | 0.00 ± 3.08 | 25.69 ± 3.65 | 4.10 ± 2.32 | 12.27 ± 1.63 | 9.43 ±2.67 |
| Heart | 0.00 ± 0.91 | 7.78 ± 1.62 | −0.01 ± 3.31 | 5.37 ± 1.71 | 2.84 ± 4.73 |
| White Adipose Tissue | 0.00 ± 9.41 | 12.60 ± 8.08 | 9.00 ± 4.12 | −8.30 ± 3.71 | −1.23 ± 7.03 |

In a separate experiment, 5 groups of 6 male SCID mice at 10 weeks of age were administered an isotype negative control antibody subcutaneously at 10 mg/kg, and H4H1657N2 at 0.1, 0.75, 2.5, or 10 mg/kg, according to the following dosing schedule: 2×per week on week 1 and 1×per week thereafter for a total 28 days of treatment. Results are summarized in Table 24, expressed as percent increase over negative control±standard deviation.

TABLE 24

| | Isotype | | | | |
|---|---|---|---|---|---|
| | Control | H4H1657N2 | | | |
| Dose | 10 mg/kg | 0.1 mg/kg | 0.75 mg/kg | 2.5 mg/kg | 10 mg/kg |
| Body Weight | 0.00 ± 0.55 | 4.24 ± 0.42 | 2.84 ± 1.03 | 6.87 ± 1.21 | 7.23 ± 1.66 |
| TA Muscle | 0.00 ± 3.89 | 3.10 ± 2.06 | 4.20 ± 3.78 | 18.63 ± 2.52 | 11.54 ± 2.15 |
| GA Muscle | 0.00 ± 1.65 | 2.75 ± 0.82 | 0.71 ± 3.15 | 15.86 ± 2.11 | 18.28 ± 2.84 |
| Quad Muscle | 0.00 ± 3.00 | 0.84 ± 1.69 | −0.62 ± 1.75 | 15.01 ± 1.37 | 16.87 ± 2.85 |
| Heart | 0.00 ± 2.24 | 6.51 ± 1.53 | 1.35 ± 2.85 | 3.25 ± 1.21 | 3.08 ± 2.66 |
| White Adipose | 0.00 ± 13.65 | −7.75 ± 8.36 | −7.81 ± 10.76 | −13.53 ± 8.00 | −28.33 ± 4.51 |
| Tissue | | | | | |

As shown in this Example, antibodies H4H1657N2 and H4H1669P produced significant increases in muscle mass when administered to mice over a range of doses.

### Example 11. Effect of Anti-GDF8 Antibodies on Glucose Homeostasis

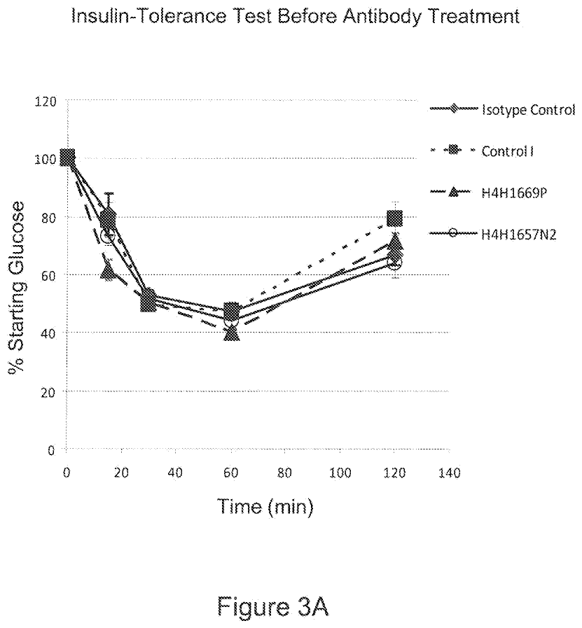

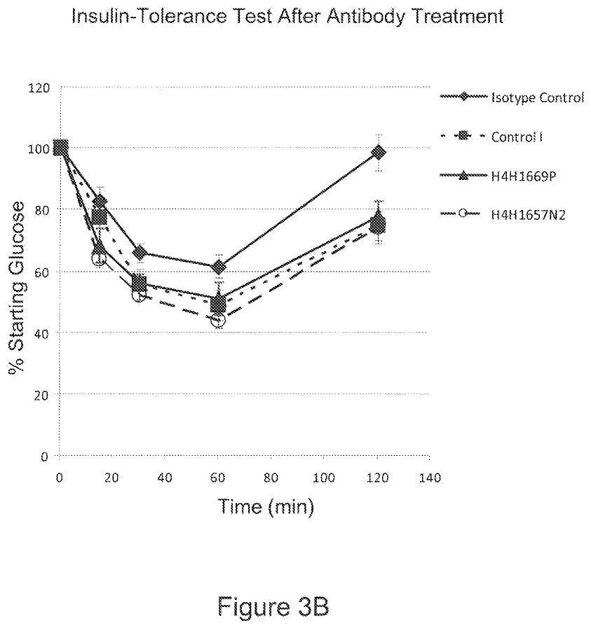

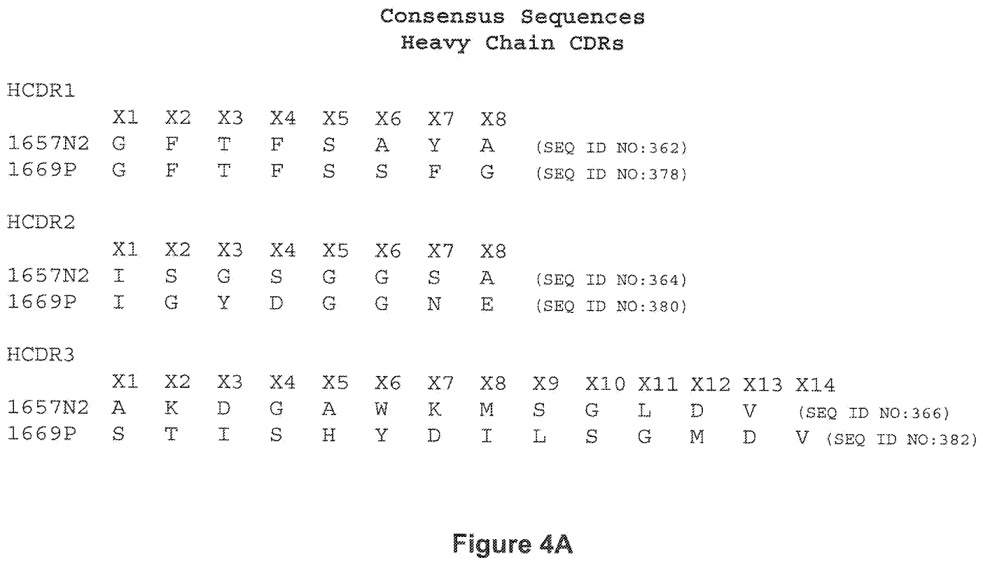

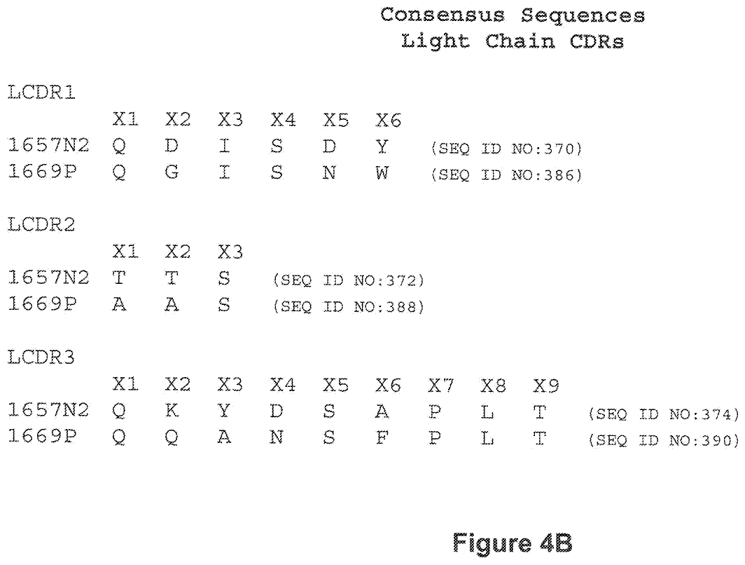

Anti-GDF8 antibodies of the invention were tested for their effects on glucose homeostasis and insulin sensitivity in a diet-induced-obesity (DIO) mouse model. In this experiment, DIO mice were obtained by feeding C57BL6 mice a high-fat diet (45% kcal fat) for 7 weeks starting at 9 weeks of age. Starting at week 8, antibodies were administered at 30 mg/kg twice a week for two more weeks, and the study was terminated a week later (21 days post-treatment). The antibodies used in this experiment were H4H1669P and H4H1657N2, as well as an isotype negative control antibody and Control I (anti-GDF8 antibody corresponding to Myo29). Insulin-tolerance tests were performed before and after antibody treatment. Insulin (2 IU/kg) was administered by intraperitoneal injection following a 4 hour fast and glucose levels were measured. The results are illustrated in FIGS. 3A (prior to antibody treatment) and 3B (after antibody treatment).

As demonstrated in this experiment, myostatin inhibition by administration of an anti-GDF8 antibody improved glucose homeostasis in DIO mice. A significant difference in glucose lowering in response to an insulin bolus was noted between the anti-GDF8 antibody groups (H4H1669P, H4H1657N2 or Control I), and the isotype control antibody group.

### Example 12. In Vivo Blockade of Muscle Atrophy by Administration of H4H1657N2

C57 mice were used to determine the in vivo properties of H4H1657N2 in preventing muscle atrophy induced by casting/immobilization and dexamethasone administration.

In the casting example, three groups of 8 C57 mice were anesthetized, and the ankle joint was immobilized at a 90° angle with casting material for 14 days. A fourth group was left unperturbed and used as a non-immobilized group control. During the 14 days of immobilization, mice were administered with an isotype negative control antibody; Control I (Myo29); or H4H1657N2. The three groups were injected subcutaneously with antibody at 30 mg/kg, 2×per week for two weeks, starting at the time of the immobilization. Results are summarized in Table 25, expressed as percent change over negative control ±standard deviation. The results showed that after 14 days of immobilization, the group that received treatment with H4H1657N2 antibody showed a significant reduction in skeletal muscle loss versus the isotype control group.

TABLE 25
(Muscle Atrophy Induced by Immobilization)

| | Non-Immobilized | Immobilized | | |
|---|---|---|---|---|
| | Isotype Control | Isotype Control | Control I | H4H1657N2 |
| TA Muscle | 0.00 ± 1.74 | −32.97 ± 4.98 | −23.36 ± 5.80 | −13.77 ± 4.23 |
| GA Muscle | 0.00 ± 1.85 | −24.34 ± 4.95 | −7.64 ± 0.97 | 3.91 ± 6.02 |

In the dexamethasone example, two groups of 5 C57 mice were anesthetized and implanted with osmotic pumps subcutaneously that delivered 1 μg/g/day of dexamethasone. Two additional groups were implanted with osmotic pumps that delivered saline and were used as controls. During the 14 days of treatment, two groups of mice (one saline and one dexamethasone) were administered an isotype negative control antibody; and two groups of mice (one saline and one dexamethasone) were administered the H4H1657N2 antibody. Antibodies were injected subcutaneously at 30 mg/kg, 2×per week for two weeks. Antibody treatment started at the time of the pump implantation. Results are summarized in Table 26, expressed as percent change over negative control±standard deviation. Comparison of the dexamethasone group that received the H4H1657N2 antibody versus the group that received the isotype control indicates that treatment with the H4H1657N2 antibody prevents the loss of muscle weights induced by dexamethasone treatment.

TABLE 26
(Muscle Atrophy Induced by Dexamethasone)

| | Saline Treated | | Dexamethasone Treated | |
|---|---|---|---|---|
| | Isotype | | Isotype | |
| | Control | H4H1657N2 | Control | H4H1657N2 |
| TA Muscle | 0.00 ± 2.73 | 14.63 ± 1.35 | −17.78 ± 2.11 | 3.25 ± 2.09 |
| GA Muscle | 0.00 ± 3.02 | 20.73 ± 1.38 | −18.97 ± 2.11 | −5.08 ± 2.07 |
| Quad Muscle | 0.00 ± 3.91 | 20.46 ± 2.40 | −23.94 ± 2.68 | −4.26 ± 2.34 |

### Example 13. Specificity of H4H1657N2 in vivo

To examine the specificity of H4H1657N2 in vivo, C57BL6 mice were injected with drug, and serum from treated mice was subjected to a mass spectrometry based ligand “fishing” experiment. Briefly, drug (H4H1657N2, Control IV or isotype negative control) was injected multiple times (Day 0, D3, D7, D10) over a 14-day period into C57BL6 mice. Animals were sacrificed on D14 and serum was incubated with anti human Fc beads. The beads were washed and bound material was eluted with SDS-PAGE loading buffer. Eluted material was subjected to SDS-PAGE and gel slices corresponding to a molecular weight range of 5-20 kDa were excised. Samples were processed for mass spectrometry using standard reduction, alkylation and trypsinization conditions. Digests were separated on a nano C18 column and automatically spotted onto Bruker Anchor Targets. MALDI-MS (MS/MS) analysis (Bruker Ultraflextreme) was performed in an automated fashion, using LC-WARP (Bruker Daltonics). The mass spectra were searched using MASCOT (Matrix Science) and results were evaluated relative to the isotype control. The eluted proteins are listed in Table 27.

TABLE 27

| Antibody Administered: | H4H1657N2 | Control IV |
|---|---|---|
| Protein(s) Eluted: | GDF8 | GDF8 |
| | | GDF11 |
| | | Inhibin beta A chain |
| | | Inhibin beta B chain |
| | | Inhibin beta C chain |

As shown in Table 27, only mouse GDF8 was identified as a binding partner for H4H1657N2 by this experiment. (The sequence of mouse and human GDF8 are identical.) By contrast, Control IV bound several other members of the TGF beta ligand family besides GDF8, including, inter alia, GDF11. This experiment confirms the specificity of H4H1657N2 for GDF8 in an in vivo context.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

1-18. (canceled)

19. An antibody or antigen-binding fragment thereof, which specifically binds to, or blocks the biological activity of wild-type mature human growth and differentiation factor-8 (GDF8), comprising:
a first amino acid sequence comprising three heavy chain complementarity determining region (HCDR) amino acid sequences: HCDR1 having SEQ ID NO: 228, HCDR2 having SEQ ID NO:230, and HCDR3 having SEQ ID NO: 232; and
a second amino acid sequence comprising three light chain complementarity determining region (LCDR) amino acid sequences: LCDR1 having SEQ ID NO: 236, LCDR2 having SEQ ID NO: 238, and LCDR3 having SEQ ID NO: 240.

20. The antibody or antigen-binding fragment of claim 19, wherein the wild-type mature human growth and differentiation factor-8 (GDF8) comprises an amino acid sequence comprising SEQ ID NO: 340.

21. (canceled)

22. (canceled)

23. A pharmaceutical composition comprising the antibody or antigen-binding fragment of claim 19; and a pharmaceutically acceptable carrier.

24. The antibody or antigen-binding fragment of claim 19, comprising: a heavy chain variable region (HCVR) comprising the amino acid sequence of SEQ ID NO: 226.

25. The antibody or antigen-binding fragment of claim 19, comprising: a light chain variable region (LCVR) comprising the amino acid sequence of SEQ ID NO: 234.

26. The antibody or antigen-binding fragment thereof of claim 19, wherein
the first amino acid sequence comprises a heavy chain variable region (HCVR) of SEQ ID NO: 226, or which differs from the HCVR of SEQ ID NO: 226 by only a single conservative amino acid substitution; and
the second amino acid sequence comprises a light chain variable region (LCVR) of SEQ ID NO: 234, or which differs from the LCVR of SEQ ID NO: 234 by only a single conservative amino acid substitution.

27. The antibody or antigen-binding fragment of claim 26, wherein the wild-type mature human growth and differentiation factor-8 (GDF8) comprises an amino acid sequence comprising SEQ ID NO: 340.

28. (canceled)

29. (canceled)

30. A pharmaceutical composition comprising the antibody or antigen-binding fragment of claim 26; and a pharmaceutically acceptable carrier.

31. An antibody or antigen-binding fragment thereof, which specifically binds to wild-type mature human growth and differentiation factor-8 (GDF8), wherein the antibody or antigen-binding fragment comprises
a heavy chain variable region (HCVR) comprising HCDR1, HCDR2 and HCDR3 domains comprising the amino acid sequences of SEQ ID NOs: 228, 230, and 232, respectively, and
a light chain variable region (LCVR) comprising LCDR1, LCDR2 and LCDR3 domains comprising the amino acid sequences of SEQ ID NOs: 236, 238, and 240, respectively, and
wherein
the heavy chain comprises the amino acid sequence of SEQ ID NO: 226, or an amino acid sequence which differs by only a single amino acid substitution from SEQ ID NO: 226; and
the light chain comprises: the amino acid sequence of SEQ ID NO: 234, or an amino acid sequence which differs by only a single amino acid substitution from SEQ ID NO: 234, and
wherein the antibody or antigen-binding fragment has at least the same binding affinity and specificity for GDF8 as an antibody or antigen-binding fragment thereof which comprises the amino acid sequences of SEQ ID NO: 226 and SEQ ID NO: 234.

32. The antibody or antigen-binding fragment of claim 31, wherein the wild-type mature human growth and differentiation factor-8 (GDF8) comprises an amino acid sequence comprising SEQ ID NO: 340.

33. (canceled)

34. (canceled)

35. A pharmaceutical composition comprising the antibody or antigen-binding fragment of claim 31; and a pharmaceutically acceptable carrier.

36-41. (canceled)

42. A method of treating a disease or disorder associated with GDF8, comprising administering to a subject in need thereof an anti-GDF8 antibody or antigen-binding fragment thereof, wherein the anti-GDF8 antibody or antigen-binding fragment thereof comprises
three heavy chain complementarity determining region (HCDR) amino acid sequences: HCDR1 having SEQ ID NO: 228, HCDR2 having SEQ ID NO:230, and HCDR3 having SEQ ID NO: 232; and
three light chain complementarity determining region (LCDR) amino acid sequences: LCDR1 having SEQ ID NO: 236, LCDR2 having SEQ ID NO: 238, and LCDR3 having SEQ ID NO: 240.

43. The method of claim 42, wherein the anti-GDF8 antibody or antigen-binding fragment thereof comprises a heavy chain variable region (HCVR) comprising the amino acid sequence of SEQ ID NO: 226 and a light chain variable region (LCVR) comprising the amino acid sequence of SEQ ID NO: 234.

44. The method of claim 42, wherein the disease or disorder associated with GDF8 is selected from the group consisting of bone loss, metabolic syndrome, muscle wasting, muscle loss, muscle atrophy, muscle wasting syndrome, sarcopenia, and cachexia.

45. The method of claim 44, wherein the bone loss is associated with one or more of the group consisting of osteoporosis, osteopenia, osteoarthritis and bone fractures.

46. The method of claim 44, wherein the muscle atrophy or wasting is caused by or associated with one or more of the group consisting of disuse, immobilization, bed rest, injury, medical treatment, surgical intervention, and mechanical ventilation.

47. The method of claim 44, wherein the metabolic syndrome is selected from the group consisting of diabetes, obesity, nutritional disorders, organ atrophy, chronic obstructive pulmonary disease, and anorexia.

48. The method of claim 42, wherein the anti-GDF8 antibody or antigen-binding fragment thereof is administered in combination with at least one additional therapeutically active component.

49. The method of claim 48, wherein the at least one additional therapeutically active component is selected from the group consisting of small molecule inhibitors of GDF8, additional GDF8 antibodies or binding molecules, growth factor inhibitors, immunosuppressants, anti-inflammatory agents, metabolic inhibitors, enzyme inhibitors, and cytotoxic/cytostatic agents.

50. The method of claim 48, wherein the at least one additional therapeutically active component is administered prior to, concurrent with, or after the administration of the anti-GDF8 antibody or antigen-binding fragment thereof.


SEQUENCE LISTING (391 sequences)

SEQ ID NO: 1 - DNA, 366 bp, synthetic construct
  misc_feature 1..366: note = Synthetic
  source 1..366: mol_type = other DNA
caggtgcagctggtgcagtctgggggaggcttggtccagcctggggggtccctgagactctcctgtgcagcctctggattcacctttaacacctatgccataagctgggtccgccaggctccagggaaggggctggaatgggtctcaactattactggtagtggttataacacatactacgcagactccgtgaagggccggttcaccatctccagagacaattccaagaacacactgtatctacagatgagcagcctgagagccgaggacacggccgtattttactgtgcgaaagactctcggtataactggaattacggaatttttgactactggggccagggaaccacggtcaccgtctcctca

SEQ ID NO: 2 - protein, 122 aa, synthetic construct
  REGION 1..122: note = Synthetic
QVQLVQSGGGLVQPGGSLRLSCAASGFTFNTYAISWVRQAPGKGLEWVSTITGSGYNTYYADSVKGRFTISRDNSKNTLYLQMSSLRAEDTAVFYCAKDSRYNWNYGIFDYWGQGTTVTVSS

SEQ ID NO: 3 - DNA, 24 bp, synthetic construct
  misc_feature 1..24: note = Synthetic
  source 1..24: mol_type = other DNA
ggattcacctttaacacctatgcc

SEQ ID NO: 4 - protein, 8 aa, synthetic construct
  REGION 1..8: note = Synthetic
GFTFNTYA

SEQ ID NO: 5 - DNA, 24 bp, synthetic construct
  misc_feature 1..24: note = Synthetic
  source 1..24: mol_type = other DNA
attactggtagtggttataacaca

SEQ ID NO: 6 - protein, 8 aa, synthetic construct
  REGION 1..8: note = Synthetic
ITGSGYNT

SEQ ID NO: 7 - DNA, 45 bp, synthetic construct
  misc_feature 1..45: note = Synthetic
  source 1..45: mol_type = other DNA
gcgaaagactctcggtataactggaattacggaatttttgactac

SEQ ID NO: 8 - protein, 15 aa, synthetic construct
  REGION 1..15: note = Synthetic
AKDSRYNWNYGIFDY

SEQ ID NO: 9 - DNA, 324 bp, synthetic construct
  misc_feature 1..324: note = Synthetic
  source 1..324: mol_type = other DNA
gacatccagatgacccagtctccagccaccctgtctgtgtctccaggggaaagagccaccctctcctgcagggccagtcagagtgttagcagcaacttagcctggtaccatcagaaacctggccaggctcccaggctcctcatctatggtgtatccaccagggccactggtatcccagccaggttcagtggcaatgggtctgggacagagttcactctcaccatcagcagcctgcagtctgaagattttgcagtttattactgtcagcagcataataactggccgctcactttcggcggagggaccaaggtggagatcaaacga

SEQ ID NO: 10 - protein, 108 aa, synthetic construct
  REGION 1..108: note = Synthetic
DIQMTQSPATLSVSPGERATLSCRASQSVSSNLAWYHQKPGQAPRLLIYGVSTRATGIPARFSGNGSGTEFTLTISSLQSEDFAVYYCQQHNNWPLTFGGGTKVEIKR

SEQ ID NO: 11 - DNA, 18 bp, synthetic construct
  misc_feature 1..18: note = Synthetic
  source 1..18: mol_type = other DNA
cagagtgttagcagcaac

SEQ ID NO: 12 - protein, 6 aa, synthetic construct
  REGION 1..6: note = Synthetic
QSVSSN

SEQ ID NO: 13-14 - skipped (2 entries)

SEQ ID NO: 15 - DNA, 27 bp, synthetic construct
  misc_feature 1..27: note = Synthetic
  source 1..27: mol_type = other DNA
cagcagcataataactggccgctcact

SEQ ID NO: 16 - protein, 9 aa, synthetic construct
  REGION 1..9: note = Synthetic
QQHNNWPLT

SEQ ID NO: 17 - DNA, 375 bp, synthetic construct
  misc_feature 1..375: note = Synthetic
  source 1..375: mol_type = other DNA
caggtgcagctggtgcagtcgggcccaggactggtgaagccttcggagaccctgtccctcacctgcactgtctctggtggctccatcagcaatagtaattactactggggctggatccgccagcccccagggaaggggctggagtggattgggactacctattatagtgggaccacctactacaacccgtccctcaagagtcgagtcaccatatccgtagacacgtccaagaaccagttctccctgaagctgagctctgtgaccgccgcagacacggctgtgtattactgtgcgagagattattatgatagtagtggttattattacaactggttcgatccctggggccagggaaccacggtcaccgtctcctca

SEQ ID NO: 18 - protein, 125 aa, synthetic construct
  REGION 1..125: note = Synthetic
QVQLVQSGPGLVKPSETLSLTCTVSGGSISNSNYYWGWIRQPPGKGLEWIGTTYYSGTTYYNPSLKSRVTISVDTSKNQFSLKLSSVTAADTAVYYCARDYYDSSGYYYNWFDPWGQGTTVTVSS

SEQ ID NO: 19 - DNA, 30 bp, synthetic construct
  misc_feature 1..30: note = Synthetic
  source 1..30: mol_type = other DNA
ggtggctccatcagcaatagtaattactac

SEQ ID NO: 20 - protein, 10 aa, synthetic construct
  REGION 1..10: note = Synthetic
GGSISNSNYY

SEQ ID NO: 21 - DNA, 21 bp, synthetic construct
  misc_feature 1..21: note = Synthetic
  source 1..21: mol_type = other DNA
acctattatagtgggaccacc

SEQ ID NO: 22 - protein, 7 aa, synthetic construct
  REGION 1..7: note = Synthetic
TYYSGTT

SEQ ID NO: 23 - DNA, 51 bp, synthetic construct
  misc_feature 1..51: note = Synthetic
  source 1..51: mol_type = other DNA
gcgagagattattatgatagtagtggttattattacaactggttcgatccc

SEQ ID NO: 24 - protein, 17 aa, synthetic construct
  REGION 1..17: note = Synthetic
ARDYYDSSGYYYNWFDP

SEQ ID NO: 25 - DNA, 324 bp, synthetic construct
  misc_feature 1..324: note = Synthetic
  source 1..324: mol_type = other DNA
gacatccagttgacccagtctccatcctccctgtctgcatctgtaggagacagagtcaccatcacttgccgggcaagtcaggacattagaaatgatttaggctggtatcagcagaaaccagggaaagcccctaagcgcctgatctatgctgcatccagtttgcaaagtggggtcccatcaaggttcagcggcagtggatctgggacagaattcagtctcacactcagcagcctgcagcctgaagattttgcaacttatttctgtctacagcatcatatttacccgtggacgttcggccaagggaccaagctggagatcaaacga

SEQ ID NO: 26 - protein, 108 aa, synthetic construct
  REGION 1..108: note = Synthetic
DIQLTQSPSSLSASVGDRVTITCRASQDIRNDLGWYQQKPGKAPKRLIYAASSLQSGVPSRFSGSGSGTEFSLTLSSLQPEDFATYFCLQHHIYPWTFGQGTKLEIKR

SEQ ID NO: 27 - DNA, 18 bp, synthetic construct
  misc_feature 1..18: note = Synthetic
  source 1..18: mol_type = other DNA
caggacattagaaatgat

SEQ ID NO: 28 - protein, 6 aa, synthetic construct
  REGION 1..6: note = Synthetic
QDIRND

SEQ ID NO: 29-30 - skipped (2 entries)

SEQ ID NO: 31 - DNA, 27 bp, synthetic construct
  misc_feature 1..27: note = Synthetic
  source 1..27: mol_type = other DNA
ctacagcatcatatttacccgtggacg

SEQ ID NO: 32 - protein, 9 aa, synthetic construct
  REGION 1..9: note = Synthetic
LQHHIYPWT

SEQ ID NO: 33 - DNA, 375 bp, synthetic construct
  misc_feature 1..375: note = Synthetic
  source 1..375: mol_type = other DNA
caagttcagctggtggagtcgggcccaggactggtgaagccttcggagaccctgtccctcacctgcactgtctatggtggctccatcagcagtggtaattactactggggctggatccgccagcccccagggaagggactggagtggattgggactatctattatagtggaagcgcctactacaacccgtccctcaagagtcgagtcaccatgtccgtagacacgtccaagaaccagttctccctgaaactgagctctgtgaccgccgcagacacggctgtttattactgtgtgagagattactatgatagtagtggtcattattacaactggttcgacccctggggccagggaaccacggtcaccgtctcctca

SEQ ID NO: 34 - protein, 125 aa, synthetic construct
  REGION 1..125: note = Synthetic
QVQLVESGPGLVKPSETLSLTCTVYGGSISSGNYYWGWIRQPPGKGLEWIGTIYYSGSAYYNPSLKSRVTMSVDTSKNQFSLKLSSVTAADTAVYYCVRDYYDSSGHYYNWFDPWGQGTTVTVSS

SEQ ID NO: 35 - DNA, 30 bp, synthetic construct
  misc_feature 1..30: note = Synthetic
  source 1..30: mol_type = other DNA
ggtggctccatcagcagtggtaattactac

SEQ ID NO: 36 - protein, 10 aa, synthetic construct
  REGION 1..10: note = Synthetic
GGSISSGNYY

SEQ ID NO: 37 - DNA, 21 bp, synthetic construct
  misc_feature 1..21: note = Synthetic
  source 1..21: mol_type = other DNA
atctattatagtggaagcgcc

SEQ ID NO: 38 - protein, 7 aa, synthetic construct
  REGION 1..7: note = Synthetic
IYYSGSA

SEQ ID NO: 39 - DNA, 51 bp, synthetic construct
  misc_feature 1..51: note = Synthetic
  source 1..51: mol_type = other DNA
gtgagagattactatgatagtagtggtcattattacaactggttcgacccc

SEQ ID NO: 40 - protein, 17 aa, synthetic construct
  REGION 1..17: note = Synthetic
VRDYYDSSGHYYNWFDP

SEQ ID NO: 41 - DNA, 324 bp, synthetic construct
  misc_feature 1..324: note = Synthetic
  source 1..324: mol_type = other DNA
gccatccagatgacccagtctccatcctccctgtctgcatctgtaggagacagagtcaccatcacttgccgggcaagtcaggacattagacatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttacccgtggacgttcggccaagggaccaaggtggagatcaaacga

SEQ ID NO: 42 - protein, 108 aa, synthetic construct
  REGION 1..108: note = Synthetic
AIQMTQSPSSLSASVGDRVTITCRASQDIRHDLGWYQQKPGKAPKRLIYAASSLQSGVPSRFSGSGSGTEFTLTISSLQPEDFATYYCLQHNTYPWTFGQGTKVEIKR

SEQ ID NO: 43 - DNA, 18 bp, synthetic construct
  misc_feature 1..18: note = Synthetic
  source 1..18: mol_type = other DNA
caggacattagacatgat

SEQ ID NO: 44 - protein, 6 aa, synthetic construct
  REGION 1..6: note = Synthetic
QDIRHD

SEQ ID NO: 45-46 - skipped (2 entries)

SEQ ID NO: 47 - DNA, 27 bp, synthetic construct
  misc_feature 1..27: note = Synthetic
  source 1..27: mol_type = other DNA
ctacagcataatacttacccgtggacg

SEQ ID NO: 48 - protein, 9 aa, synthetic construct
  REGION 1..9: note = Synthetic
LQHNTYPWT

SEQ ID NO: 49 - DNA, 357 bp, synthetic construct
  misc_feature 1..357: note = Synthetic
  source 1..357: mol_type = other DNA
caggtgcagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatcgtggatacacctttggggttgactactggggccagggaaccacggtcaccgtctcctca

SEQ ID NO: 50 - protein, 119 aa, synthetic construct
  REGION 1..119: note = Synthetic
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYTFGVDYWGQGTTVTVSS

SEQ ID NO: 51 - DNA, 24 bp, synthetic construct
  misc_feature 1..24: note = Synthetic
  source 1..24: mol_type = other DNA
ggattcaccttcagtagctatagc

SEQ ID NO: 52 - protein, 8 aa, synthetic construct
  REGION 1..8: note = Synthetic
GFTFSSYS

SEQ ID NO: 53 - DNA, 24 bp, synthetic construct
  misc_feature 1..24: note = Synthetic
  source 1..24: mol_type = other DNA
attagtagtagtagtagttacata

SEQ ID NO: 54 - protein, 8 aa, synthetic construct
  REGION 1..8: note = Synthetic
ISSSSSYI

SEQ ID NO: 55 - DNA, 36 bp, synthetic construct
  misc_feature 1..36: note = Synthetic
  source 1..36: mol_type = other DNA
gcgagagatcgtggatacacctttggggttgactac

SEQ ID NO: 56 - protein, 12 aa, synthetic construct
  REGION 1..12: note = Synthetic
ARDRGYTFGVDY

SEQ ID NO: 57 - DNA, 324 bp, synthetic construct
  misc_feature 1..324: note = Synthetic
  source 1..324: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaaggtggagatcaaacga

SEQ ID NO: 58 - protein, 108 aa, synthetic construct
  REGION 1..108: note = Synthetic
AIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKVEIKR

SEQ ID NO: 59 - DNA, 18 bp, synthetic construct
  misc_feature 1..18: note = Synthetic
  source 1..18: mol_type = other DNA
cagggcattagaaatgat

SEQ ID NO: 60 - protein, 6 aa, synthetic construct
  REGION 1..6: note = Synthetic
QGIRND

SEQ ID NO: 61-62 - skipped (2 entries)

SEQ ID NO: 63 - DNA, 27 bp, synthetic construct
  misc_feature 1..27: note = Synthetic
  source 1..27: mol_type = other DNA
ctacagcataatagttacccgtacact

SEQ ID NO: 64 - protein, 9 aa, synthetic construct
  REGION 1..9: note = Synthetic
LQHNSYPYT

SEQ ID NO: 65 - DNA, 375 bp, synthetic construct
  misc_feature 1..375: note = Synthetic
  source 1..375: mol_type = other DNA
caggtgcagctggtgcagtcgggcccaggactggtgaagccttcggagaccctgtccctcacctgcactgtctctggtggctccatcatcacttatagttactactggggctggatccgccagcccccagggaaggggctggagtggattgggactatccatcatagtgggagcacctactacaacccgtccctcaagagtcgagtcaccatatccgtagacacgtccaagaaccagttctccctgacactgagttctgtgaccgccgcagacacggctgtgtattactgtgcgagagactactatgatagtagtggttattattataactggttcgacccctggggccagggaaccatggtcaccgtctcctca

SEQ ID NO: 66 - protein, 125 aa, synthetic construct
  REGION 1..125: note = Synthetic
QVQLVQSGPGLVKPSETLSLTCTVSGGSIITYSYYWGWIRQPPGKGLEWIGTIHHSGSTYYNPSLKSRVTISVDTSKNQFSLTLSSVTAADTAVYYCARDYYDSSGYYYNWFDPWGQGTMVTVSS

SEQ ID NO: 67 - DNA, 30 bp, synthetic construct
  misc_feature 1..30: note = Synthetic
  source 1..30: mol_type = other DNA
ggtggctccatcatcacttatagttactac

SEQ ID NO: 68 - protein, 10 aa, synthetic construct
  REGION 1..10: note = Synthetic
GGSIITYSYY

SEQ ID NO: 69 - DNA, 21 bp, synthetic construct
  misc_feature 1..21: note = Synthetic
  source 1..21: mol_type = other DNA
atccatcatagtgggagcacc

SEQ ID NO: 70 - protein, 7 aa, synthetic construct
  REGION 1..7: note = Synthetic
IHHSGST

SEQ ID NO: 71 - DNA, 51 bp, synthetic construct
  misc_feature 1..51: note = Synthetic
  source 1..51: mol_type = other DNA
gcgagagactactatgatagtagtggttattattataactggttcgacccc

SEQ ID NO: 72 - protein, 17 aa, synthetic construct
  REGION 1..17: note = Synthetic
ARDYYDSSGYYYNWFDP

SEQ ID NO: 73 - DNA, 324 bp, synthetic construct
  misc_feature 1..324: note = Synthetic
  source 1..324: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttccccgtggacgttcggccaagggaccaaggtggagatcaaacga

SEQ ID NO: 74 - protein, 108 aa, synthetic construct
  REGION 1..108: note = Synthetic
AIQMTQSPSSLSASVGDRVTITCRASQGIRNDLGWYQQKPGKAPKRLIYAASSLQSGVPSRFSGSGSGTEFTLTISSLQPEDFATYYCLQHNSSPWTFGQGTKVEIKR

SEQ ID NO: 75 - DNA, 18 bp, synthetic construct
  misc_feature 1..18: note = Synthetic
  source 1..18: mol_type = other DNA
cagggcattagaaatgat

SEQ ID NO: 76 - protein, 6 aa, synthetic construct
  REGION 1..6: note = Synthetic
QGIRND

SEQ ID NO: 77-78 - skipped (2 entries)

SEQ ID NO: 79 - DNA, 27 bp, synthetic construct
  misc_feature 1..27: note = Synthetic
  source 1..27: mol_type = other DNA
ctacagcataatagttccccgtggacg

SEQ ID NO: 80 - protein, 9 aa, synthetic construct
  REGION 1..9: note = Synthetic
LQHNSSPWT

SEQ ID NO: 81 - DNA, 369 bp, synthetic construct
  misc_feature 1..369: note = Synthetic
  source 1..369: mol_type = other DNA
caggtgcacttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaatgatgataagcgctacagcccatctctgaagagcaggctcaccatcaccaaggacacctccaaaaaccaggtggtccttacaatgaccaacatggaccctgtggacacagccacatattactgtacacacacctcccgttataactggcactacggcttccttgactactggggccagggaaccacggtcaccgtctcctca

SEQ ID NO: 82 - protein, 123 aa, synthetic construct
  REGION 1..123: note = Synthetic
QVHLKESGPTLVKPTQTLTLTCTFSGFSLSTSGVGVGWIRQPPGKALEWLALIYWNDDKRYSPSLKSRLTITKDTSKNQVVLTMTNMDPVDTATYYCTHTSRYNWHYGFLDYWGQGTTVTVSS

SEQ ID NO: 83 - DNA, 30 bp, synthetic construct
  misc_feature 1..30: note = Synthetic
  source 1..30: mol_type = other DNA
gggttctcactcagcactagtggagtgggt

SEQ ID NO: 84 - protein, 10 aa, synthetic construct
  REGION 1..10: note = Synthetic
GFSLSTSGVG

SEQ ID NO: 85 - DNA, 21 bp, synthetic construct
  misc_feature 1..21: note = Synthetic
  source 1..21: mol_type = other DNA
atttattggaatgatgataag

SEQ ID NO: 86 - protein, 7 aa, synthetic construct
  REGION 1..7: note = Synthetic
IYWNDDK

SEQ ID NO: 87 - DNA, 45 bp, synthetic construct
  misc_feature 1..45: note = Synthetic
  source 1..45: mol_type = other DNA
acacacacctcccgttataactggcactacggcttccttgactac

SEQ ID NO: 88 - protein, 15 aa, synthetic construct
  REGION 1..15: note = Synthetic
THTSRYNWHYGFLDY

SEQ ID NO: 89 - DNA, 324 bp, synthetic construct
  misc_feature 1..324: note = Synthetic
  source 1..324: mol_type = other DNA
gccatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacaatataatagttacccgctcactttcggcggagggaccaaggtggaaatcaaacga

SEQ ID NO: 90 - protein, 108 aa, synthetic construct
  REGION 1..108: note = Synthetic
AIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIKR

SEQ ID NO: 91 - DNA, 18 bp, synthetic construct
  misc_feature 1..18: note = Synthetic
  source 1..18: mol_type = other DNA
cagggcattagcaattat

SEQ ID NO: 92 - protein, 6 aa, synthetic construct
  REGION 1..6: note = Synthetic
QGISNY

SEQ ID NO: 93-94 - skipped (2 entries)

SEQ ID NO: 95 - DNA, 27 bp, synthetic construct
  misc_feature 1..27: note = Synthetic
  source 1..27: mol_type = other DNA
caacaatataatagttacccgctcact

SEQ ID NO: 96 - protein, 9 aa, synthetic construct
  REGION 1..9: note = Synthetic
QQYNSYPLT

SEQ ID NO: 97 - DNA, 375 bp, synthetic construct
  misc_feature 1..375: note = Synthetic
  source 1..375: mol_type = other DNA
gaggtgcagctggtgcagttgggcccaggactggtgaagccttcggagaccctgtccctcacctgcactgtctctggtggctccatcagcaatagtaattactactggggctggatccgccagcccccagggaaggggctggagtggattgggactacctattatagtgggaccacctactacaacccgtccctcaagagtcgagtcaccatatccgtagacacgtccaagaaccagttctccctgaagctgagctctgtgaccgccgcagacacggctgtgtattactgtgcgagagattattatgatagtagtggttattattacaactggttcgatccctggggccagggaaccacggtcaccgtctcctca

SEQ ID NO: 98 - protein, 125 aa, synthetic construct
  REGION 1..125: note = Synthetic
EVQLVQLGPGLVKPSETLSLTCTVSGGSISNSNYYWGWIRQPPGKGLEWIGTTYYSGTTYYNPSLKSRVTISVDTSKNQFSLKLSSVTAADTAVYYCARDYYDSSGYYYNWFDPWGQGTTVTVSS

SEQ ID NO: 99 - DNA, 30 bp, synthetic construct; identical to SEQ ID NO: 19
  misc_feature 1..30: note = Synthetic
  source 1..30: mol_type = other DNA

SEQ ID NO: 100 - protein, 10 aa, synthetic construct
  REGION 1..10: note = Synthetic
GGSISNSNYY

SEQ ID NO: 101 - DNA, 21 bp, synthetic construct
  misc_feature 1..21: note = Synthetic
  source 1..21: mol_type = other DNA
acctattatagtgggaccacc

SEQ ID NO: 102 - protein, 7 aa, synthetic construct
  REGION 1..7: note = Synthetic
TYYSGTT

SEQ ID NO: 103 - DNA, 51 bp, synthetic construct; identical to SEQ ID NO: 23
  misc_feature 1..51: note = Synthetic
  source 1..51: mol_type = other DNA

SEQ ID NO: 104 - protein, 17 aa, synthetic construct
  REGION 1..17: note = Synthetic
ARDYYDSSGYYYNWFDP

SEQ ID NO: 105 - DNA, 324 bp, synthetic construct
  misc_feature 1..324: note = Synthetic
  source 1..324: mol_type = other DNA
gccatccagatgacccagtctccatcctccctgtctgcatctgtaggagacagagtcaccatcacttgccgggcaagtcaggacattagaaatgatttaggctggtatcagcagaaaccagggaaagcccctaagcgcctgatctatgctgcatccagtttgcaaagtggggtcccatcaaggttcagcggcagtggatctgggacagaattcagtctcacactcagcagcctgcagcctgaagattttgcaacttatttctgtctacagcatcatatttacccgtggacgttcggccaagggaccaaggtggagatcaaacga

SEQ ID NO: 106 - protein, 108 aa, synthetic construct
  REGION 1..108: note = Synthetic
AIQMTQSPSSLSASVGDRVTITCRASQDIRNDLGWYQQKPGKAPKRLIYAASSLQSGVPSRFSGSGSGTEFSLTLSSLQPEDFATYFCLQHHIYPWTFGQGTKVEIKR

SEQ ID NO: 107 - DNA, 18 bp, synthetic construct
  misc_feature 1..18: note = Synthetic
  source 1..18: mol_type = other DNA
caggacattagaaatgat

SEQ ID NO: 108 - protein, 6 aa, synthetic construct
  REGION 1..6: note = Synthetic
QDIRND

SEQ ID NO: 109-110 - skipped (2 entries)

SEQ ID NO: 111 - DNA, 27 bp, synthetic construct
  misc_feature 1..27: note = Synthetic
  source 1..27: mol_type = other DNA
ctacagcatcatatttacccgtggacg

SEQ ID NO: 112 - protein, 9 aa, synthetic construct
  REGION 1..9: note = Synthetic
LQHHIYPWT

SEQ ID NO: 113 - DNA, 355 bp, synthetic construct
  misc_feature 1..355: note = Synthetic
  source 1..355: mol_type = other DNA
caggtgcagctggtggagtctgggggaggcgtggtccagcctgggaggtccctgagactctcctgtgcagcctctggattcaccttcagtcgctatggcattcactgggtccgccaggctccaggcaaggggctggagtgggtggctgttatatcttatgatggaagtgatgaatactatgtagactccgtgaagggccgattcagcatctcccgagacaattccaagaacacgctttatctacaaatgaacagtctgaggcctgcggactcggctgtttattactgtgtgaaaggagatctggaacttggttttgactactggggccagggaaccctggtcaccgtctcctcaa

SEQ ID NO: 114 - protein, 118 aa, synthetic construct
  REGION 1..118: note = Synthetic
QVQLVESGGGVVQPGRSLRLSCAASGFTFSRYGIHWVRQAPGKGLEWVAVISYDGSDEYYVDSVKGRFSISRDNSKNTLYLQMNSLRPADSAVYYCVKGDLELGFDYWGQGTLVTVSS

SEQ ID NO: 115 - DNA, 24 bp, synthetic construct
  misc_feature 1..24: note = Synthetic
  source 1..24: mol_type = other DNA
ggattcaccttcagtcgctatggc

SEQ ID NO: 116 - protein, 8 aa, synthetic construct
  REGION 1..8: note = Synthetic
GFTFSRYG

SEQ ID NO: 117 - DNA, 24 bp, synthetic construct
  misc_feature 1..24: note = Synthetic
  source 1..24: mol_type = other DNA
atatcttatgatggaagtgatgaa

SEQ ID NO: 118 - protein, 8 aa, synthetic construct
  REGION 1..8: note = Synthetic
ISYDGSDE

SEQ ID NO: 119 - DNA, 33 bp, synthetic construct
  misc_feature 1..33: note = Synthetic
  source 1..33: mol_type = other DNA
gtgaaaggagatctggaacttggttttgactac

SEQ ID NO: 120 - protein, 11 aa, synthetic construct
  REGION 1..11: note = Synthetic
VKGDLELGFDY

SEQ ID NO: 121 - DNA, 337 bp, synthetic construct
  misc_feature 1..337: note = Synthetic
  source 1..337: mol_type = other DNA
gatattgtgatgactcaggctgcaccctctatacctgtcattccaggagagtcagtatccatgtcctgcaggtctagtaagagtctcctgtacagtaatggacatacttacgtgtattggtttgtgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaaaatctagaatttccgctcacgttcggtgctgggaccaagctggagctgaaac

SEQ ID NO: 122 - protein, 112 aa, synthetic construct
  REGION 1..112: note = Synthetic
DIVMTQAAPSIPVIPGESVSMSCRSSKSLLYSNGHTYVYWFVQRPGQSPQLLIYRMSNLASGVPDRFSGSGSGTAFTLRISRVEAEDVGVYYCMQNLEFPLTFGAGTKLELK

SEQ ID NO: 123 - DNA, 33 bp, synthetic construct
  misc_feature 1..33: note = Synthetic
  source 1..33: mol_type = other DNA
aagagtctcctgtacagtaatggacatacttac

SEQ ID NO: 124 - protein, 11 aa, synthetic construct
  REGION 1..11: note = Synthetic
KSLLYSNGHTY

SEQ ID NO: 125-126 - skipped (2 entries)

SEQ ID NO: 127 - DNA, 27 bp, synthetic construct
  misc_feature 1..27: note = Synthetic
  source 1..27: mol_type = other DNA
atgcaaaatctagaatttccgctcacg

SEQ ID NO: 128 - protein, 9 aa, synthetic construct
  REGION 1..9: note = Synthetic
MQNLEFPLT

SEQ ID NO: 129 - DNA, 355 bp, synthetic construct
  misc_feature 1..355: note = Synthetic
  source 1..355: mol_type = other DNA
caggtgcagctggtggaggcggggggaggcgtggtccagcctgggaggtccctgagactctcctgtgcagcctctggattcaccttcagtcgctatggcattcactgggtccgccaggctccaggcaaggggctggagtgggtggctgttatatcttatgatggaactgatgaatactatgcagactccgtgaagggccgattcaccatctcccgagacaattccaagaacacgctttatctacaaatgaacagtctgagacctgcggactcggctgtatattactgtgcgaaaggagatctggaacttggttttgactactggggccagggaaccctggtcaccgtctcctcaa

SEQ ID NO: 130 - protein, 118 aa, synthetic construct
  REGION 1..118: note = Synthetic
QVQLVEAGGGVVQPGRSLRLSCAASGFTFSRYGIHWVRQAPGKGLEWVAVISYDGTDEYYADSVKGRFTISRDNSKNTLYLQMNSLRPADSAVYYCAKGDLELGFDYWGQGTLVTVSS

SEQ ID NO: 131 - DNA, 24 bp, synthetic construct
  misc_feature 1..24: note = Synthetic
  source 1..24: mol_type = other DNA
ggattcaccttcagtcgctatggc

SEQ ID NO: 132 - protein, 8 aa, synthetic construct
  REGION 1..8: note = Synthetic
GFTFSRYG

SEQ ID NO: 133 - DNA, 24 bp, synthetic construct
  misc_feature 1..24: note = Synthetic
  source 1..24: mol_type = other DNA
atatcttatgatggaactgatgaa

SEQ ID NO: 134 - protein, 8 aa, synthetic construct
  REGION 1..8: note = Synthetic
ISYDGTDE

SEQ ID NO: 135 - DNA, 33 bp, synthetic construct
  misc_feature 1..33: note = Synthetic
  source 1..33: mol_type = other DNA
gcgaaaggagatctggaacttggttttgactac

SEQ ID NO: 136 - protein, 11 aa, synthetic construct
  REGION 1..11: note = Synthetic
AKGDLELGFDY

SEQ ID NO: 137 - DNA, 322 bp, synthetic construct
  misc_feature 1..322: note = Synthetic
  source 1..322: mol_type = other DNA
gccatccggttgacccagtctccatcttccgtgtctgcatctgtgggagacagagtcaccatcacttgtcgggcgagtcaggatattagtatttggttagcctggtatcagcagagtccagggaaagcccctaaactcctgatcaatgttgcatcccgtttgcaaagtggggtcccatcaaggttcagcggcagtggatctgggacagatttcactctcaccatcaacggtctgcagcctgaagattttgtaacttactattgtcaacaggctaacagtttcccgatcaccttcggccaagggacacgactggcgaccaaac

SEQ ID NO: 138 - protein, 107 aa, synthetic construct
  REGION 1..107: note = Synthetic
AIRLTQSPSSVSASVGDRVTITCRASQDISIWLAWYQQSPGKAPKLLINVASRLQSGVPSRFSGSGSGTDFTLTINGLQPEDFVTYYCQQANSFPITFGQGTRLATK

SEQ ID NO: 139 - DNA, 18 bp, synthetic construct
  misc_feature 1..18: note = Synthetic
  source 1..18: mol_type = other DNA
caggatattagtatttgg

SEQ ID NO: 140 - protein, 6 aa, synthetic construct
  REGION 1..6: note = Synthetic
QDISIW

SEQ ID NO: 141-142 - skipped (2 entries)

SEQ ID NO: 143 - DNA, 27 bp, synthetic construct
  misc_feature 1..27: note = Synthetic
  source 1..27: mol_type = other DNA
caacaggctaacagtttcccgatcacc

SEQ ID NO: 144 - protein, 9 aa, synthetic construct
  REGION 1..9: note = Synthetic
QQANSFPIT

SEQ ID NO: 145 - DNA, 358 bp, synthetic construct
  misc_feature 1..358: note = Synthetic
  source 1..358: mol_type = other DNA
caggtgcagctggtgcagtctgggggaggcctggtcaagcctggggggtccctgagactctcctgtgcagcctctggattcatcttcaatacctataccatgaattgggtccgccaggctccagggaaggggctggagtgggtctcatccatcactagtcgtggtacttatatattctactcagactcacttaagggccgattcaccatttccagagacaacgccaataactcactgtttctgcaaatgaacagcctgagagtcgaagacacggctgtttattactgttcgagagatcgtggatacacctttggtcctgactactggggccagggaaccctggtcaccgtctcttcag

SEQ ID NO: 146 - protein, 119 aa, synthetic construct
  REGION 1..119: note = Synthetic
QVQLVQSGGGLVKPGGSLRLSCAASGFIFNTYTMNWVRQAPGKGLEWVSSITSRGTYIFYSDSLKGRFTISRDNANNSLFLQMNSLRVEDTAVYYCSRDRGYTFGPDYWGQGTLVTVSS

SEQ ID NO: 147 - DNA, 24 bp, synthetic construct
  misc_feature 1..24: note = Synthetic
  source 1..24: mol_type = other DNA
ggattcatcttcaatacctatacc

SEQ ID NO: 148 - protein, 8 aa, synthetic construct
  REGION 1..8: note = Synthetic
GFIFNTYT

SEQ ID NO: 149 - DNA, 24 bp, synthetic construct
  misc_feature 1..24: note = Synthetic
  source 1..24: mol_type = other DNA
atcactagtcgtggtacttatata

SEQ ID NO: 150 - protein, 8 aa, synthetic construct
  REGION 1..8: note = Synthetic
ITSRGTYI

SEQ ID NO: 151 - DNA, 36 bp, synthetic construct
  misc_feature 1..36: note = Synthetic
  source 1..36: mol_type = other DNA
tcgagagatcgtggatacacctttggtcctgactac

SEQ ID NO: 152 - protein, 12 aa, synthetic construct
  REGION 1..12: note = Synthetic
SRDRGYTFGPDY

SEQ ID NO: 153 - DNA, 322 bp, synthetic construct
  misc_feature 1..322: note = Synthetic
  source 1..322: mol_type = other DNA
gacatccagatgacccagtctccatcctccctgtctgcatctgtaggagacagagtcaccatcacttgccgggcaagtcaggacattagaaatgatttaggctggtatcagcagaaaccagggaaagcccctaagggcctgatctatgctgcatccagtttgcaaagtggggtcccatcaaggttcagcggcagtggatctgggacagaattcactctcacaatcagcagcctgcagcctgaagattttgcaacttatcactgtctacattatgattttcatcctcggacgttcggccaagggaccaaggtggaaatcaagc

SEQ ID NO: 154 - protein, 107 aa, synthetic construct
  REGION 1..107: note = Synthetic
DIQMTQSPSSLSASVGDRVTITCRASQDIRNDLGWYQQKPGKAPKGLIYAASSLQSGVPSRFSGSGSGTEFTLTISSLQPEDFATYHCLHYDFHPRTFGQGTKVEIK

SEQ ID NO: 155 - DNA, 18 bp, synthetic construct
  misc_feature 1..18: note = Synthetic
  source 1..18: mol_type = other DNA
caggacattagaaatgat

SEQ ID NO: 156 - protein, 6 aa, synthetic construct
  REGION 1..6: note = Synthetic
QDIRND

SEQ ID NO: 157-158 - skipped (2 entries)

SEQ ID NO: 159 - DNA, 27 bp, synthetic construct
  misc_feature 1..27: note = Synthetic
  source 1..27: mol_type = other DNA
ctacattatgattttcatcctcggacg

SEQ ID NO: 160 - protein, 9 aa, synthetic construct
  REGION 1..9: note = Synthetic
LHYDFHPRT

SEQ ID NO: 161 - DNA, 391 bp, synthetic construct
  misc_feature 1..391: note = Synthetic
  source 1..391: mol_type = other DNA
caggtgcaactggtggagtctgggggaggcgtggtccagcctgggaggtccctgagactctcctgtgcagcgtctggattcaccttcagtagctatgccatgcactgggtccgccaggctccaggcaaggggctggagtgggtggcagttatatggtatgatggaactaattattactatgcagactccgtgaagggccgattcaccatctccagagacaattccaagaacacgctgtatctgcaaatgaacagcctgagagccgaggacacggctgtgtattactgtgcgagagatcccctatattacgatattttgactggttattcccccgactactactacggtatggacgtctggggccaagggaccacggtcaccgtctcctcag

SEQ ID NO: 162 - protein, 130 aa, synthetic construct
  REGION 1..130: note = Synthetic
QVQLVESGGGVVQPGRSLRLSCAASGFTFSSYAMHWVRQAPGKGLEWVAVIWYDGTNYYYADSVKGRFTISRDNSKNTLYLQMNSLRAEDTAVYYCARDPLYYDILTGYSPDYYYGMDVWGQGTTVTVSS

SEQ ID NO: 163 - DNA, 24 bp, synthetic construct
  misc_feature 1..24: note = Synthetic
  source 1..24: mol_type = other DNA
ggattcaccttcagtagctatgcc

SEQ ID NO: 164 - protein, 8 aa, synthetic construct
  REGION 1..8: note = Synthetic
GFTFSSYA

SEQ ID NO: 165 - DNA, 24 bp, synthetic construct
  misc_feature 1..24: note = Synthetic
  source 1..24: mol_type = other DNA
atatggtatgatggaactaattat

SEQ ID NO: 166 - protein, 8 aa, synthetic construct
  REGION 1..8: note = Synthetic
IWYDGTNY

SEQ ID NO: 167 - DNA, 69 bp, synthetic construct
  misc_feature 1..69: note = Synthetic
  source 1..69: mol_type = other DNA
gcgagagatcccctatattacgatattttgactggttattcccccgactactactacggtatggacgtc

SEQ ID NO: 168 - protein, 23 aa, synthetic construct
  REGION 1..23: note = Synthetic
ARDPLYYDILTGYSPDYYYGMDV

SEQ ID NO: 169 - DNA, 322 bp, synthetic construct
  misc_feature 1..322: note = Synthetic
  source 1..322: mol_type = other DNA
gaaatagtgatgacgcagtctccagccaccctgtctgtgtctccaggggaaagagccaccctctcctgcagggccagtcagacttttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataagtggccgctcactttcggcggagggaccaaggtggagatcaaac

SEQ ID NO: 170 - protein, 107 aa, synthetic construct
  REGION 1..107: note = Synthetic
EIVMTQSPATLSVSPGERATLSCRASQTFSSNLAWYQQKPGQAPRLLIYGASTRATGIPARFSGSGSGTEFTLTISSLQSEDFAVYYCQQYNKWPLTFGGGTKVEIK

SEQ ID NO: 171 - DNA, 18 bp, synthetic construct
  misc_feature 1..18: note = Synthetic
  source 1..18: mol_type = other DNA
cagacttttagcagcaac

SEQ ID NO: 172 - protein, 6 aa, synthetic construct
  REGION 1..6: note = Synthetic
QTFSSN

SEQ ID NO: 173-174 - skipped (2 entries)

SEQ ID NO: 175 - DNA, 27 bp, synthetic construct
  misc_feature 1..27: note = Synthetic
  source 1..27: mol_type = other DNA
cagcagtataataagtggccgctcact

SEQ ID NO: 176 - protein, 9 aa, synthetic construct
  REGION 1..9: note = Synthetic
QQYNKWPLT

SEQ ID NO: 177 - DNA, 367 bp, synthetic construct
  misc_feature 1..367: note = Synthetic
  source 1..367: mol_type = other DNA
gaggtgcagctgttggagtctgggggaggcttggtccagcctggggagtccctgagactctcctgtgcagcctctggattcacctttagcagctatgccatgaactgggtccgccaggctccagggaaggggctggagtgggtctcaactatcagtggtagtggtggttatatatactacgcagactccgtgaagggccggttcaccatctccagagacaattccaagaacacgctgtatctgcaaatgaacagcctgagagccgaagacacggccgtatatttctgtgcgaaagattccaggtataactggaactacggcaattttgactactggggccagggaaccctggtcaccgtctcctcag

SEQ ID NO: 178 - protein, 122 aa, synthetic construct
  REGION 1..122: note = Synthetic
EVQLLESGGGLVQPGESLRLSCAASGFTFSSYAMNWVRQAPGKGLEWVSTISGSGGYIYYADSVKGRFTISRDNSKNTLYLQMNSLRAEDTAVYFCAKDSRYNWNYGNFDYWGQGTLVTVSS

SEQ ID NO: 179 - DNA, 24 bp, synthetic construct
  misc_feature 1..24: note = Synthetic
  source 1..24: mol_type = other DNA
ggattcacctttagcagctatgcc

SEQ ID NO: 180 - protein, 8 aa, synthetic construct
  REGION 1..8: note = Synthetic
GFTFSSYA

SEQ ID NO: 181 - DNA, 24 bp, synthetic construct
  misc_feature 1..24: note = Synthetic
  source 1..24: mol_type = other DNA
atcagtggtagtggtggttatata

SEQ ID NO: 182 - protein, 8 aa, synthetic construct
  REGION 1..8: note = Synthetic
ISGSGGYI

SEQ ID NO: 183 - DNA, 45 bp, synthetic construct
  misc_feature 1..45: note = Synthetic
  source 1..45: mol_type = other DNA
gcgaaagattccaggtataactggaactacggcaattttgactac

SEQ ID NO: 184 - protein, 15 aa, synthetic construct
  REGION 1..15: note = Synthetic
AKDSRYNWNYGNFDY

SEQ ID NO: 185 - DNA, 322 bp, synthetic construct
  misc_feature 1..322: note = Synthetic
  source 1..322: mol_type = other DNA
gaaatagtgatgacgcagtctccagccaccctgtctgtgtctccaggggaaagagccaccctctcctgcagggccagtcagaatgttagcagcaacttagcctggaacaagcagaaacctggccaggctcccagactcctcatctatgctacatccaccagggccactggtgtcccagccaggttcagtgccagtgggtctgggacagacttcgctctcaccatcaacagcctgcagtctgaagattttgcagtttattactgtcagcagtataataactggcctctcactttcggcggagggaccaaggtggagatcaaac

SEQ ID NO: 186 - protein, 107 aa, synthetic construct
  REGION 1..107: note = Synthetic
EIVMTQSPATLSVSPGERATLSCRASQNVSSNLAWNKQKPGQAPRLLIYATSTRATGVPARFSASGSGTDFALTINSLQSEDFAVYYCQQYNNWPLTFGGGTKVEIK

SEQ ID NO: 187 - DNA, 18 bp, synthetic construct
  misc_feature 1..18: note = Synthetic
  source 1..18: mol_type = other DNA
cagaatgttagcagcaac

SEQ ID NO: 188 - protein, 6 aa, synthetic construct
  REGION 1..6: note = Synthetic
QNVSSN

SEQ ID NO: 189-190 - skipped (2 entries)

SEQ ID NO: 191 - DNA, 27 bp, synthetic construct
  misc_feature 1..27: note = Synthetic
  source 1..27: mol_type = other DNA
cagcagtataataactggcctctcact

SEQ ID NO: 192 - protein, 9 aa, synthetic construct
  REGION 1..9: note = Synthetic
QQYNNWPLT

SEQ ID NO: 193 - DNA, 367 bp, synthetic construct
  misc_feature 1..367: note = Synthetic
  source 1..367: mol_type = other DNA
gaggtgcaactgttggaatctgggggaggcttggtacagcctggggggtccctgagactctcctgtgtagcctctcgattcaccttcagcagcaatgccatgagttgggtccgccaggctccagggacggggctggagtgggtctcagctattactggtagtggtagtaggacatactacgcagactccgtgaagggccggttcaccatctccagagacaattccaagaacacggtgtatctgcaaatgaacagcctgagaggcgaggacacggccgtatattactgtgcgaaagatcaagggggtacctggaactacggagattttgactactggggccagggaaccctggtcaccgtctcctcag

SEQ ID NO: 194 - protein, 122 aa, synthetic construct
  REGION 1..122: note = Synthetic
EVQLLESGGGLVQPGGSLRLSCVASRFTFSSNAMSWVRQAPGTGLEWVSAITGSGSRTYYADSVKGRFTISRDNSKNTVYLQMNSLRGEDTAVYYCAKDQGGTWNYGDFDYWGQGTLVTVSS

SEQ ID NO: 195 - DNA, 24 bp, synthetic construct
  misc_feature 1..24: note = Synthetic
  source 1..24: mol_type = other DNA
cgattcaccttcagcagcaatgcc

SEQ ID NO: 196 - protein, 8 aa, synthetic construct
  REGION 1..8: note = Synthetic
RFTFSSNA

SEQ ID NO: 197 - DNA, 24 bp, synthetic construct
  misc_feature 1..24: note = Synthetic
  source 1..24: mol_type = other DNA
attactggtagtggtagtaggaca

SEQ ID NO: 198 - protein, 8 aa, synthetic construct
  REGION 1..8: note = Synthetic
ITGSGSRT

SEQ ID NO: 199 - DNA, 45 bp, synthetic construct
  misc_feature 1..45: note = Synthetic
  source 1..45: mol_type = other DNA
gcgaaagatcaagggggtacctggaactacggagattttgactac

SEQ ID NO: 200 - protein, 15 aa, synthetic construct
  REGION 1..15: note = Synthetic
AKDQGGTWNYGDFDY

SEQ ID NO: 201 - DNA, 322 bp, synthetic construct
  misc_feature 1..322: note = Synthetic
  source 1..322: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtctcccagccaggttcagtggcagtgggtctgggacagacttcactctcaccatcagcagcctgcagtctgaagattttgcagtttattactgtcagcagtataataactggcctctcactttcggcggagggaccaaggtggagatcaagc

SEQ ID NO: 202 - protein, 107 aa, synthetic construct
  REGION 1..107: note = Synthetic
EIVMTQSPATLSVSPGERATLSCRASQSVSSNLAWYQQKPGQAPRLLIYGASTRATGLPARFSGSGSGTDFTLTISSLQSEDFAVYYCQQYNNWPLTFGGGTKVEIK

SEQ ID NO: 203 - DNA, 18 bp, synthetic construct
  misc_feature 1..18: note = Synthetic
  source 1..18: mol_type = other DNA
cagagtgttagcagcaac

SEQ ID NO: 204 - protein, 6 aa, synthetic construct
  REGION 1..6: note = Synthetic
QSVSSN

SEQ ID NO: 205-206 - skipped (2 entries)

SEQ ID NO: 207 - DNA, 27 bp, synthetic construct
  misc_feature 1..27: note = Synthetic
  source 1..27: mol_type = other DNA
cagcagtataataactggcctctcact

SEQ ID NO: 208 - protein, 9 aa, synthetic construct
  REGION 1..9: note = Synthetic
QQYNNWPLT

SEQ ID NO: 209 - DNA, 373 bp, synthetic construct
  misc_feature 1..373: note = Synthetic
  source 1..373: mol_type = other DNA
caggtgcagctggtggagtctgggggagacgtggtccagcctgggaggtccctgagactctcctgtacagcctctggattcaccttcagtagttatggcatgcactgggtccgccaggctccaggcagggggctggagtgggtggcagttatatcatttgatggaaaaaataaatactatgcagactccgtgaagggccgattcaccgtctccagagacaattccaagaacacgctgtttctgcaaatgaacagcctgagagctgaggacacggctctatattactgtgcgaaaaggatagcagcaactggttactactacttctacggtttggacgtctggggccaagggaccacggtcaccgtctcctcag

SEQ ID NO: 210 - protein, 124 aa, synthetic construct
  REGION 1..124: note = Synthetic
QVQLVESGGDVVQPGRSLRLSCTASGFTFSSYGMHWVRQAPGRGLEWVAVISFDGKNKYYADSVKGRFTVSRDNSKNTLFLQMNSLRAEDTALYYCAKRIAATGYYYFYGLDVWGQGTTVTVSS

SEQ ID NO: 211 - DNA, 24 bp, synthetic construct
  misc_feature 1..24: note = Synthetic
  source 1..24: mol_type = other DNA
ggattcaccttcagtagttatggc

SEQ ID NO: 212 - protein, 8 aa, synthetic construct
  REGION 1..8: note = Synthetic
GFTFSSYG

SEQ ID NO: 213 - DNA, 24 bp, synthetic construct
  misc_feature 1..24: note = Synthetic
  source 1..24: mol_type = other DNA
atatcatttgatggaaaaaataaa

SEQ ID NO: 214 - protein, 8 aa, synthetic construct
  REGION 1..8: note = Synthetic
ISFDGKNK

SEQ ID NO: 215 - DNA, 51 bp, synthetic construct
  misc_feature 1..51: note = Synthetic
  source 1..51: mol_type = other DNA
gcgaaaaggatagcagcaactggttactactacttctacggtttggacgtc

SEQ ID NO: 216 - protein, 17 aa, synthetic construct
  REGION 1..17: note = Synthetic
AKRIAATGYYYFYGLDV

SEQ ID NO: 217 - DNA, 322 bp, synthetic construct
  misc_feature 1..322: note = Synthetic
  source 1..322: mol_type = other DNA
gaaataatgatgacgcagtctccagccaccctgtctgtgtctccaggggaaagaggcaccctctcctgcagggccagtcagagtgttagtagcaacttagcctggtaccagcagaaacctggccaggctcccaggctcctcatctatggtgcatccaccagggccagtggtatcccagccaggttcagtggcagtgggtctgggacagagttcactctcaccgtcagcagcctgcagtctgaagattttgcagtttattactgtcagcagtataataactggccgctcactttcggcggagggaccaaggtggagatcaaac

SEQ ID NO: 218 - protein, 107 aa, synthetic construct
  REGION 1..107: note = Synthetic
EIMMTQSPATLSVSPGERGTLSCRASQSVSSNLAWYQQKPGQAPRLLIYGASTRASGIPARFSGSGSGTEFTLTVSSLQSEDFAVYYCQQYNNWPLTFGGGTKVEIK

SEQ ID NO: 219 - DNA, 18 bp, synthetic construct
  misc_feature 1..18: note = Synthetic
  source 1..18: mol_type = other DNA
cagagtgttagtagcaac

SEQ ID NO: 220 - protein, 6 aa, synthetic construct
  REGION 1..6: note = Synthetic
QSVSSN

SEQ ID NO: 221-222 - skipped (2 entries)

SEQ ID NO: 223 - DNA, 27 bp, synthetic construct
  misc_feature 1..27: note = Synthetic
  source 1..27: mol_type = other DNA
cagcagtataataactggccgctcact

SEQ ID NO: 224 - protein, 9 aa, synthetic construct
  REGION 1..9: note = Synthetic
QQYNNWPLT

SEQ ID NO: 225 - DNA, 367 bp, synthetic construct
  misc_feature 1..367: note = Synthetic
  source 1..367: mol_type = other DNA
gaggttcagctgttggagtctgggggaggcttggtacagccgggggggtccctgagactctcctgtgcagcctctggattcacctctatcacctatgccatgagctgggtccgccaggctccagggaaggggctggagtgggtctcagctattagtgttagtggtactaatacatactacgcagactccgtgaagggccggttcaccatctccagagacaagtccaagaacatgctgtatctgcaaatgaacagcctgagagccgaggacacggccgtgtattactgtgcgaaagatctcctacataactggaaatacgggacttttgatatctggggccaagggacaatggtcaccgtctcttcag

SEQ ID NO: 226 - protein, 122 aa, synthetic construct
  REGION 1..122: note = Synthetic
EVQLLESGGGLVQPGGSLRLSCAASGFTSITYAMSWVRQAPGKGLEWVSAISVSGTNTYYADSVKGRFTISRDKSKNMLYLQMNSLRAEDTAVYYCAKDLLHNWKYGTFDIWGQGTMVTVSS

SEQ ID NO: 227 - DNA, 24 bp, synthetic construct
  misc_feature 1..24: note = Synthetic
  source 1..24: mol_type = other DNA
ggattcacctctatcacctatgcc

SEQ ID NO: 228 - protein, 8 aa, synthetic construct
  REGION 1..8: note = Synthetic
GFTSITYA

SEQ ID NO: 229 - DNA, 24 bp, synthetic construct
  misc_feature 1..24: note = Synthetic
  source 1..24: mol_type = other DNA
attagtgttagtggtactaataca

SEQ ID NO: 230 - protein, 8 aa, synthetic construct
  REGION 1..8: note = Synthetic
ISVSGTNT

SEQ ID NO: 231 - DNA, 45 bp, synthetic construct
  misc_feature 1..45: note = Synthetic
  source 1..45: mol_type = other DNA
gcgaaagatctcctacataactggaaatacgggacttttgatatc

SEQ ID NO: 232 - protein, 15 aa, synthetic construct
  REGION 1..15: note = Synthetic
AKDLLHNWKYGTFDI

SEQ ID NO: 233 - DNA, 322 bp, synthetic construct
  misc_feature 1..322: note = Synthetic
  source 1..322: mol_type = other DNA
gaaatagtgatgacgcagtctccagccaccctgtctgtgtctccaggggaaagagccaccctctcctgcagggccagtcagagtgttgacagcaacttagtctggtaccaacaaaaacctggccaggttcccaggctcctcatctatggtgcatccaccagggccactggtatcccagccaggttcagtggcagtgggtctgggacagagttcactctcaccatcagcagcctgcagtctgaagattttgcagtttattactgtcagcagtataataagtggccgctcactttcggcggagggaccaaggtggagatcaaac

SEQ ID NO: 234 - protein, 107 aa, synthetic construct
  REGION 1..107: note = Synthetic
EIVMTQSPATLSVSPGERATLSCRASQSVDSNLVWYQQKPGQVPRLLIYGASTRATGIPARFSGSGSGTEFTLTISSLQSEDFAVYYCQQYNKWPLTFGGGTKVEIK

SEQ ID NO: 235 - DNA, 18 bp, synthetic construct
  misc_feature 1..18: note = Synthetic
  source 1..18: mol_type = other DNA
cagagtgttgacagcaac

SEQ ID NO: 236 - protein, 6 aa, synthetic construct
  REGION 1..6: note = Synthetic
QSVDSN

SEQ ID NO: 237-238 - skipped (2 entries)

SEQ ID NO: 239 - DNA, 27 bp, synthetic construct
  misc_feature 1..27: note = Synthetic
  source 1..27: mol_type = other DNA
cagcagtataataagtggccgctcact

SEQ ID NO: 240 - protein, 9 aa, synthetic construct
  REGION 1..9: note = Synthetic
QQYNKWPLT

SEQ ID NO: 241 - DNA, 367 bp, synthetic construct; identical to SEQ ID NO: 225
  misc_feature 1..367: note = Synthetic
  source 1..367: mol_type = other DNA

SEQ ID NO: 242 - protein, 122 aa, synthetic construct; identical to SEQ ID NO: 226
  REGION 1..122: note = Synthetic

SEQ ID NO: 243 - DNA, 24 bp, synthetic construct
  misc_feature 1..24: note = Synthetic
  source 1..24: mol_type = other DNA
ggattcacctctatcacctatgcc

SEQ ID NO: 244 - protein, 8 aa, synthetic construct
  REGION 1..8: note = Synthetic
GFTSITYA

SEQ ID NO: 245 - DNA, 24 bp, synthetic construct
  misc_feature 1..24: note = Synthetic
  source 1..24: mol_type = other DNA
attagtgttagtggtactaataca

SEQ ID NO: 246 - protein, 8 aa, synthetic construct
  REGION 1..8: note = Synthetic
ISVSGTNT

SEQ ID NO: 247 - DNA, 45 bp, synthetic construct; identical to SEQ ID NO: 231
  misc_feature 1..45: note = Synthetic
  source 1..45: mol_type = other DNA

SEQ ID NO: 248 - protein, 15 aa, synthetic construct
  REGION 1..15: note = Synthetic
AKDLLHNWKYGTFDI

SEQ ID NO: 249 - DNA, 325 bp, synthetic construct
  misc_feature 1..325: note = Synthetic
  source 1..325: mol_type = other DNA
gaaatagtgatgacgcagtctccagccaccctgtctgtgtctccaggggaaagagccaccctctcctgcagggccagtcagagtgttagcagcaacttagcctggaaccagcagaaacctggccaggctcccaggctcctcatctatggtgcatccaccagggccacgggtatcccagccaggttcagtggcagtgggtctgggacagagttcactctcaccatcagcagcctgcagtctgaagattttgcagtttattactgtcagcagtataacaactggcccatgtacacttttggccaggggaccaagctggagatcaaac

SEQ ID NO: 250 - protein, 108 aa, synthetic construct
  REGION 1..108: note = Synthetic
EIVMTQSPATLSVSPGERATLSCRASQSVSSNLAWNQQKPGQAPRLLIYGASTRATGIPARFSGSGSGTEFTLTISSLQSEDFAVYYCQQYNNWPMYTFGQGTKLEIK

SEQ ID NO: 251 - DNA, 18 bp, synthetic construct
  misc_feature 1..18: note = Synthetic
  source 1..18: mol_type = other DNA
cagagtgttagcagcaac

SEQ ID NO: 252 - protein, 6 aa, synthetic construct
  REGION 1..6: note = Synthetic
QSVSSN

SEQ ID NO: 253-254 - skipped (2 entries)

SEQ ID NO: 255 - DNA, 30 bp, synthetic construct
  misc_feature 1..30: note = Synthetic
  source 1..30: mol_type = other DNA
cagcagtataacaactggcccatgtacact

SEQ ID NO: 256 - protein, 10 aa, synthetic construct
  REGION 1..10: note = Synthetic
QQYNNWPMYT

SEQ ID NO: 257 - DNA, 361 bp, synthetic construct
  misc_feature 1..361: note = Synthetic
  source 1..361: mol_type = other DNA
caggtgcagctggtggagtctgggggaggcgtggtccagcctgggaggtccctgagactctcctgtgaagcctctggattcaccttcagtagttctggcatgcactgggtccgccaggctccaggcaaggggctgcagtgggtggctgttatatcatatgatggaaataataaattttatgaagactccgtgaagggccgattgaccatttccagagacaattccaacaacactctgtggctgcaaatgaacagcctgagagttgaagacacggctgtttattactgtgcgaaatcaggaggtagagtgggagccgcctttgcctactggggccagggaaccctggtcaccgtctcctcag

SEQ ID NO: 258 - protein, 120 aa, synthetic construct
  REGION 1..120: note = Synthetic
QVQLVESGGGVVQPGRSLRLSCEASGFTFSSSGMHWVRQAPGKGLQWVAVISYDGNNKFYEDSVKGRLTISRDNSNNTLWLQMNSLRVEDTAVYYCAKSGGRVGAAFAYWGQGTLVTVSS

SEQ ID NO: 259 - DNA, 24 bp, synthetic construct
  misc_feature 1..24: note = Synthetic
  source 1..24: mol_type = other DNA
ggattcaccttcagtagttctggc

SEQ ID NO: 260 - protein, 8 aa, synthetic construct
  REGION 1..8: note = Synthetic
GFTFSSSG

SEQ ID NO: 261 - DNA, 24 bp, synthetic construct
  misc_feature 1..24: note = Synthetic
  source 1..24: mol_type = other DNA
atatcatatgatggaaataataaa

SEQ ID NO: 262 - protein, 8 aa, synthetic construct
  REGION 1..8: note = Synthetic
ISYDGNNK

SEQ ID NO: 263 - DNA, 39 bp, synthetic construct
  misc_feature 1..39: note = Synthetic
  source 1..39: mol_type = other DNA
gcgaaatcaggaggtagagtgggagccgcctttgcctac

SEQ ID NO: 264 - protein, 13 aa, synthetic construct
  REGION 1..13: note = Synthetic
AKSGGRVGAAFAY

SEQ ID NO: 265 - DNA, 337 bp, synthetic construct
  misc_feature 1..337: note = Synthetic
  source 1..337: mol_type = other DNA
gatattgtgaacactcagtctccactctctctgcccgtcacccctggagagccggcctccatctcctgcaggtctagtcagagcctcctgtatggtaatggatacaactatttggattggtacctgcagaagccagggcactctccacagctcctgatctatttgggttctaatcggggctccggggtccctgacaggttcagtggcagtggatcaggcacagattttacactgaagatcagcagagtggaggctgaagatgttggcatttattactgcatgcaaactctacaaactccattcactttcggccctgggaccaaaatgtatatcaaac

SEQ ID NO: 266 - protein, 112 aa, synthetic construct
  REGION 1..112: note = Synthetic
DIVNTQSPLSLPVTPGEPASISCRSSQSLLYGNGYNYLDWYLQKPGHSPQLLIYLGSNRGSGVPDRFSGSGSGTDFTLKISRVEAEDVGIYYCMQTLQTPFTFGPGTKMYIK

SEQ ID NO: 267 - DNA, 33 bp, synthetic construct
  misc_feature 1..33: note = Synthetic
  source 1..33: mol_type = other DNA
cagagcctcctgtatggtaatggatacaactat

SEQ ID NO: 268 - protein, 11 aa, synthetic construct
  REGION 1..11: note = Synthetic
QSLLYGNGYNY

SEQ ID NO: 269-270 - skipped (2 entries)

SEQ ID NO: 271 - DNA, 27 bp, synthetic construct
  misc_feature 1..27: note = Synthetic
  source 1..27: mol_type = other DNA
atgcaaactctacaaactccattcact

SEQ ID NO: 272 - protein, 9 aa, synthetic construct
  REGION 1..9: note = Synthetic
MQTLQTPFT

SEQ ID NO: 273 - DNA, 376 bp, synthetic construct
  misc_feature 1..376: note = Synthetic
  source 1..376: mol_type = other DNA
gaggtgcagctgttggagtctgggggaggcttggtacagcctggggggtccctgagactctcctgtgcaggctctggaatcacctttagcagctatgccatgagctgggtccgccaggctccagggaaggggctggagtgggtctcagctattagtggtaatggtggtaccacaaactacgcagactccgtgaagggccggttcaccatctccagagacaactccaagaacacgctgtatctgcaaatgaacagcctgagagccgaggacacggccgtatattactgtgcgaaagaaagaatccttaccagcagctggacgaggtacggtattatggacgtctggggccaagggaccacggtcaccgtctcctcag

SEQ ID NO: 274 - protein, 125 aa, synthetic construct
  REGION 1..125: note = Synthetic
EVQLLESGGGLVQPGGSLRLSCAGSGITFSSYAMSWVRQAPGKGLEWVSAISGNGGTTNYADSVKGRFTISRDNSKNTLYLQMNSLRAEDTAVYYCAKERILTSSWTRYGIMDVWGQGTTVTVSS

SEQ ID NO: 275 - DNA, 24 bp, synthetic construct
  misc_feature 1..24: note = Synthetic
  source 1..24: mol_type = other DNA
ggaatcacctttagcagctatgcc

SEQ ID NO: 276 - protein, 8 aa, synthetic construct
  REGION 1..8: note = Synthetic
GITFSSYA

SEQ ID NO: 277 - DNA, 24 bp, synthetic construct
  misc_feature 1..24: note = Synthetic
  source 1..24: mol_type = other DNA
attagtggtaatggtggtaccaca

SEQ ID NO: 278 - protein, 8 aa, synthetic construct
  REGION 1..8: note = Synthetic
ISGNGGTT

SEQ ID NO: 279 - DNA, 54 bp, synthetic construct
  misc_feature 1..54: note = Synthetic
  source 1..54: mol_type = other DNA
gcgaaagaaagaatccttaccagcagctggacgaggtacggtattatggacgtc

SEQ ID NO: 280 - protein, 18 aa, synthetic construct
  REGION 1..18: note = Synthetic
AKERILTSSWTRYGIMDV

SEQ ID NO: 281 - DNA, 322 bp, synthetic construct
  misc_feature 1..322: note = Synthetic
  source 1..322: mol_type = other DNA
gaaatagtgatgacgcagtctccagccaccctgtctatgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctctcactttcggcggagggaccaagttagagatcaaac

SEQ ID NO: 282 - protein, 107 aa, synthetic construct
  REGION 1..107: note = Synthetic
EIVMTQSPATLSMSPGERATLSCRASQSVSSNLAWYQQKPGQAPRLLIYGASTRATGIPARFSGSGSGTEFTLTISSLQSEDFAVYYCQQYNNWPLTFGGGTKLEIK

SEQ ID NO: 283 - DNA, 18 bp, synthetic construct
  misc_feature 1..18: note = Synthetic
  source 1..18: mol_type = other DNA
cagagtgttagcagcaac

SEQ ID NO: 284 - protein, 6 aa, synthetic construct
  REGION 1..6: note = Synthetic
QSVSSN

SEQ ID NO: 285-286 - skipped (2 entries)

SEQ ID NO: 287 - DNA, 27 bp, synthetic construct
  misc_feature 1..27: note = Synthetic
  source 1..27: mol_type = other DNA
cagcagtataataactggcctctcact

SEQ ID NO: 288 - protein, 9 aa, synthetic construct
  REGION 1..9: note = Synthetic
QQYNNWPLT

SEQ ID NO: 289 - DNA, 358 bp, synthetic construct
  misc_feature 1..358: note = Synthetic
  source 1..358: mol_type = other DNA
caggtgcaactacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaaacaccaactacaacccgtccctcaagagtcgagtcaccatatcagtagacacgtccaagaaccagttctccctgaaactgaactctgtgaccgccgcggacacggctgtgtattactgtgcgagaagagaggctacagtaactccatactttgactactggggccagggaaccctggtcaccgtctcctcag

SEQ ID NO: 290 - protein, 119 aa, synthetic construct
  REGION 1..119: note = Synthetic
QVQLQQWGAGLLKPSETLSLTCAVYGGSFSGYYWSWIRQPPGKGLEWIGEINHSGNTNYNPSLKSRVTISVDTSKNQFSLKLNSVTAADTAVYYCARREATVTPYFDYWGQGTLVTVSS

SEQ ID NO: 291 - DNA, 24 bp, synthetic construct
  misc_feature 1..24: note = Synthetic
  source 1..24: mol_type = other DNA
ggtgggtccttcagtggttactac

SEQ ID NO: 292 - protein, 8 aa, synthetic construct
  REGION 1..8: note = Synthetic
GGSGSGYY

SEQ ID NO: 293 - DNA, 21 bp, synthetic construct
  misc_feature 1..21: note = Synthetic
  source 1..21: mol_type = other DNA
atcaatcatagtggaaacacc

SEQ ID NO: 294 - protein, 7 aa, synthetic construct
  REGION 1..7: note = Synthetic
INHSGNT

SEQ ID NO: 295 - DNA, 39 bp, synthetic construct
  misc_feature 1..39: note = Synthetic
  source 1..39: mol_type = other DNA
gcgagaagagaggctacagtaactccatactttgactac

SEQ ID NO: 296 - protein, 13 aa, synthetic construct
  REGION 1..13: note = Synthetic
ARREATVTPYFDY

SEQ ID NO: 297 - DNA, 322 bp, synthetic construct
  misc_feature 1..322: note = Synthetic
  source 1..322: mol_type = other DNA
gacatccagttgacccagtctccatccttcctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttatccgctcactttcggcggagggaccaaggtggagatcaaac

SEQ ID NO: 298 - protein, 107 aa, synthetic construct
  REGION 1..107: note = Synthetic
DIQLTQSPSFLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 299 - DNA, 18 bp, synthetic construct
  misc_feature 1..18: note = Synthetic
  source 1..18: mol_type = other DNA
cagggcattagcagttat

SEQ ID NO: 300 - protein, 6 aa, synthetic construct
  REGION 1..6: note = Synthetic
QGISSY

SEQ ID NO: 301-302 - skipped (2 entries)

SEQ ID NO: 303 - DNA, 27 bp, synthetic construct
  misc_feature 1..27: note = Synthetic
  source 1..27: mol_type = other DNA
caacagcttaatagttatccgctcact

SEQ ID NO: 304 - protein, 9 aa, synthetic construct
  REGION 1..9: note = Synthetic
QQLNSYPLT

SEQ ID NO: 305 - DNA, 355 bp, synthetic construct
  misc_feature 1..355: note = Synthetic
  source 1..355: mol_type = other DNA
caggtgcagctggtggagtctgggggaggcgtggtccagcctgggaggtccctgagactctcctgtgtagtctctggattcaacttcagtaggaatggcatacactgggtccgccaggctccaggcaaggggctggaatgggtggcagttatatcatatgatggaagaaataaattttatgtagagtccgtgaagggccgattcaccatctccagagacaattccaagaacacgctgtatctgcaaatgaacagtctgagagttgaggacacggctgtatattactgtgcgaaatcctcaattggagggttttttgaatactggggccagggaaccctggtcaccgtctcctcag

SEQ ID NO: 306 - protein, 118 aa, synthetic construct
  REGION 1..118: note = Synthetic
QVQLVESGGGVVQPGRSLRLSCVVSGFNFSRNGIHWVRQAPGKGLEWVAVISYDGRNKFYVESVKGRFTISRDNSKNTLYLQMNSLRVEDTAVYYCAKSSIGGFFEYWGQGTLVTVSS

SEQ ID NO: 307 - DNA, 24 bp, synthetic construct
  misc_feature 1..24: note = Synthetic
  source 1..24: mol_type = other DNA
ggattcaacttcagtaggaatggc

SEQ ID NO: 308 - protein, 8 aa, synthetic construct
  REGION 1..8: note = Synthetic
GFNFSRNG

SEQ ID NO: 309 - DNA, 24 bp, synthetic construct
  misc_feature 1..24: note = Synthetic
  source 1..24: mol_type = other DNA
atatcatatgatggaagaaataaa

SEQ ID NO: 310 - protein, 8 aa, synthetic construct
  REGION 1..8: note = Synthetic
ISYDGRNK

SEQ ID NO: 311 - DNA, 33 bp, synthetic construct
  misc_feature 1..33: note = Synthetic
  source 1..33: mol_type = other DNA
gcgaaatcctcaattggagggttttttgaatac

SEQ ID NO: 312 - protein, 11 aa, synthetic construct
  REGION 1..11: note = Synthetic
AKSSIGGFFEY

SEQ ID NO: 313 - DNA, 337 bp, synthetic construct
  misc_feature 1..337: note = Synthetic
  source 1..337: mol_type = other DNA
gatattgtgatgactcagtctccactctccctgcccgtcactcctggagagccggcctccatctcctgcaggtctagtcagagcctcctgcatagtaatggatacaactatttggattggtacctgcagaagccaggacagtctccacaactcatgatctatttgggttctcatcgggcctccggggtccctgacaggttcagtggcagtggatcaggcacagattttacactgaaaatcagcagagtggaggctgaggatgttggagtctattactgcattcaagttcaacaaactccgatcaccttcggccaagggacacggctggagattaaac

SEQ ID NO: 314 - protein, 112 aa, synthetic construct
  REGION 1..112: note = Synthetic
DIVMTQSPLSLPVTPGEPASISCRSSQSLLHSNGYNYLDWYLQKPGQSPQLMIYLGSHRASGVPDRFSGSGSGTDFTLKISRVEAEDVGVYYCIQVQQTPITFGQGTRLEIK

SEQ ID NO: 315 - DNA, 33 bp, synthetic construct
  misc_feature 1..33: note = Synthetic
  source 1..33: mol_type = other DNA
cagagcctcctgcatagtaatggatacaactat

SEQ ID NO: 316 - protein, 11 aa, synthetic construct
  REGION 1..11: note = Synthetic
QSLLHSNGYNY

SEQ ID NO: 317-318 - skipped (2 entries)

SEQ ID NO: 319 - DNA, 27 bp, synthetic construct
  misc_feature 1..27: note = Synthetic
  source 1..27: mol_type = other DNA
attcaagttcaacaaactccgatcacc

SEQ ID NO: 320 - protein, 9 aa, synthetic construct
  REGION 1..9: note = Synthetic
IQVQQTPIT

SEQ ID NO: 321 - DNA, 337 bp, synthetic construct
  misc_feature 1..337: note = Synthetic
  source 1..337: mol_type = other DNA
gatattgtgatgactcagactccactctcctcacctgtcacccttggacagccggcctccatctcctgcaggtctagtcaaagcctcgtacacagtgatggaaacacctacttgagttggcttcagcagaggccaggccagcctccaagactcctaatttacaagatttctaaccggttctctggggtcccagacagattcagtggcagtggggcagggacagatttcacactgaaaatcagcagggtggaagctgaggatgtcggggtttatttctgcatgcaagctacacaatttccgtacacttttggccaggggaccaagctggagatcaaag

SEQ ID NO: 322 - protein, 112 aa, synthetic construct
  REGION 1..112: note = Synthetic
DIVMTQTPLSSPVTLGQPASISCRSSQSLVHSDGNTYLSWLQQRPGQPPRLLIYKISNRFSGVPDRFSGSGAGTDFTLKISRVEAEDVGVYFCMQATQFPYTFGQGTKLEIK

SEQ ID NO: 323 - DNA, 33 bp, synthetic construct
  misc_feature 1..33: note = Synthetic
  source 1..33: mol_type = other DNA
caaagcctcgtacacagtgatggaaacacctac

SEQ ID NO: 324 - protein, 11 aa, synthetic construct
  REGION 1..11: note = Synthetic
QSLVHSDGNTY

SEQ ID NO: 325-326 - skipped (2 entries)

SEQ ID NO: 327 - DNA, 27 bp, synthetic construct
  misc_feature 1..27: note = Synthetic
  source 1..27: mol_type = other DNA
atgcaagctacacaatttccgtacact

SEQ ID NO: 328 - protein, 9 aa, synthetic construct
  REGION 1..9: note = Synthetic
MQATQFPYT

SEQ ID NO: 329-334 - skipped (6 entries)

SEQ ID NO: 335 - protein, 330 aa, synthetic construct [sequence omitted]

SEQ ID NO: 336 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 337 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 338 - DNA, 1128 bp, Homo sapiens
  source 1..1128: mol_type = genomic DNA
atgcaaaaactgcaactctgtgtttatatttacctgtttatgctgattgttgctggtccagtggatctaaatgagaacagtgagcaaaaagaaaatgtggaaaaagaggggctgtgtaatgcatgtacttggagacaaaacactaaatcttcaagaatagaagccattaagatacaaatcctcagtaaacttcgtctggaaacagctcctaacatcagcaaagatgttataagacaacttttacccaaagctcctccactccgggaactgattgatcagtatgatgtccagagggatgacagcagcgatggctctttggaagatgacgattatcacgctacaacggaaacaatcattaccatgcctacagagtctgattttctaatgcaagtggatggaaaacccaaatgttgcttctttaaatttagctctaaaatacaatacaataaagtagtaaaggcccaactatggatatatttgagacccgtcgagactcctacaacagtgtttgtgcaaatcctgagactcatcaaacctatgaaagacggtacaaggtatactggaatccgatctctgaaacttgacatgaacccaggcactggtatttggcagagcattgatgtgaagacagtgttgcaaaattggctcaaacaacctgaatccaacttaggcattgaaataaaagctttagatgagaatggtcatgatcttgctgtaaccttcccaggaccaggagaagatgggctgaatccgtttttagaggtcaaggtaacagacacaccaaaaagatccagaagggattttggtcttgactgtgatgagcactcaacagaatcacgatgctgtcgttaccctctaactgtggattttgaagcttttggatgggattggattatcgctcctaaaagatataaggccaattactgctctggagagtgtgaatttgtatttttacaaaaatatcctcatactcatctggtacaccaagcaaaccccagaggttcagcaggcccttgctgtactcccacaaagatgtctccaattaatatgctatattttaatggcaaagaacaaataatatatgggaaaattccagcgatggtagtagaccgctgtgggtgctcatga

SEQ ID NO: 339 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 340 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 341 - DNA, 1224 bp, Homo sapiens
  source 1..1224: mol_type = genomic DNA
atggtgctcgcggccccgctgctgctgggcttcctgctcctcgccctggagctgcggccccggggggaggcggccgagggccccgcggcggcggcggcggcggcggcggcggcggcagcggcgggggtcgggggggagcgctccagccggccagccccgtccgtggcgcccgagccggacggctgccccgtgtgcgtttggcggcagcacagccgcgagctgcgcctagagagcatcaagtcgcagatcttgagcaaactgcggctcaaggaggcgcccaacatcagccgcgaggtggtgaagcagctgctgcccaaggcgccgccgctgcagcagatcctggacctacacgacttccagggcgacgcgctgcagcccgaggacttcctggaggaggacgagtaccacgccaccaccgagaccgtcattagcatggcccaggagacggacccagcagtacagacagatggcagccctctctgctgccattttcacttcagccccaaggtgatgttcacaaaggtactgaaggcccagctgtgggtgtacctacggcctgtaccccgcccagccacagtctacctgcagatcttgcgactaaaacccctaactggggaagggaccgcagggggagggggcggaggccggcgtcacatccgtatccgctcactgaagattgagctgcactcacgctcaggccattggcagagcatcgacttcaagcaagtgctacacagctggttccgccagccacagagcaactggggcatcgagatcaacgcctttgatcccagtggcacagacctggctgtcacctccctggggccgggagccgaggggctgcatccattcatggagcttcgagtcctagagaacacaaaacgttcccggcggaacctgggtctggactgcgacgagcactcaagcgagtcccgctgctgccgatatcccctcacagtggactttgaggctttcggctgggactggatcatcgcacctaagcgctacaaggccaactactgctccggccagtgcgagtacatgttcatgcaaaaatatccgcatacccatttggtgcagcaggccaatccaagaggctctgctgggccctgttgtacccccaccaagatgtccccaatcaacatgctctacttcaatgacaagcagcagattatctacggcaagatccctggcatggtggtggatcgctgtggctgctcttaa

SEQ ID NO: 342 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 343 - protein, 109 aa, Homo sapiens
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 344 - DNA, 1503 bp, Homo sapiens
  source 1..1503: mol_type = genomic DNA
atgagactccccaaactcctcactttcttgctttggtacctggcttggctggacctggaattcatctgcactgtgttgggtgcccctgacttgggccagagaccccaggggaccaggccaggattggccaaagcagaggccaaggagaggccccccctggcccggaacgtcttcaggccagggggtcacagctatggtgggggggccaccaatgccaatgccagggcaaagggaggcaccgggcagacaggaggcctgacacagcccaagaaggatgaacccaaaaagctgccccccagaccgggcggccctgaacccaagccaggacaccctccccaaacaaggcaggctacagcccggactgtgaccccaaaaggacagcttcccggaggcaaggcacccccaaaagcaggatctgtccccagctccttcctgctgaagaaggccagggagcccgggcccccacgagagcccaaggagccgtttcgcccaccccccatcacaccccacgagtacatgctctcgctgtacaggacgctgtccgatgctgacagaaagggaggcaacagcagcgtgaagttggaggctggcctggccaacaccatcaccagctttattgacaaagggcaagatgaccgaggtcccgtggtcaggaagcagaggtacgtgtttgacattagtgccctggagaaggatgggctgctgggggccgagctgcggatcttgcggaagaagccctcggacacggccaagccagcggcccccggaggcgggcgggctgcccagctgaagctgtccagctgccccagcggccggcagccggcctccttgctggatgtgcgctccgtgccaggcctggacggatctggctgggaggtgttcgacatctggaagctcttccgaaactttaagaactcggcccagctgtgcctggagctggaggcctgggaacggggcagggccgtggacctccgtggcctgggcttcgaccgcgccgcccggcaggtccacgagaaggccctgttcctggtgtttggccgcaccaagaaacgggacctgttctttaatgagattaaggcccgctctggccaggacgataagaccgtgtatgagtacctgttcagccagcggcgaaaacggcgggccccactggccactcgccagggcaagcgacccagcaagaaccttaaggctcgctgcagtcggaaggcactgcatgtcaacttcaaggacatgggctgggacgactggatcatcgcaccccttgagtacgaggctttccactgcgaggggctgtgcgagttcccattgcgctcccacctggagcccacgaatcatgcagtcatccagaccctgatgaactccatggaccccgagtccacaccacccacctgctgtgtgcccacgcggctgagtcccatcagcatcctcttcattgactctgccaacaacgtggtgtataagcagtatgaggacatggtcgtggagtcgtgtggctgcagg

SEQ ID NO: 345 - protein, 501 aa, Homo sapiens
MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPASLLDVRSVPGLDGSGWEVFDIWKLFRNFKNSAQLCLELEAWERGRAVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 346 - protein, 120 aa, Homo sapiens
APLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 347 - protein, 107 aa, synthetic construct
  REGION 1..107: note = Synthetic
QAKHKQRKRLKSSCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 348 - protein, 109 aa, synthetic construct
  REGION 1..109: note = Synthetic
ALDTNYCFSSTEKNCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 349 - protein, 111 aa, synthetic construct
  REGION 1..111: note = Synthetic
DFGLDCDEHSTESRCCKRHPLYVDFSDVGWNDWIVAPPGYHAFYCSGECEFVFLQKYPHTHLVHQANPRGSAGPCCTPTKMSPINMLYFNGKEQIIYGKIPAMVVDRCGCS

SEQ ID NO: 350 - protein, 110 aa, synthetic construct
  REGION 1..110: note = Synthetic
DFGLDCDEHSTESRCCVRQLYIDFRKDLGWKWIHEPKGYHANFCSGECEFVFLQKYPHTHLVHQANPRGSAGPCCTPTKMSPINMLYFNGKEQIIYGKIPAMVVDRCGCS

SEQ ID NO: 351 - protein, 114 aa, synthetic construct
  REGION 1..114: note = Synthetic
DFGLDCDEHSTESRCCRYPLTVDFEAFGWDWIIAPKRYKANYCSGECPFPLADHLNSTNHAIVQTLVNSVNSKIPKACCTPTKMSPINMLYFNGKEQIIYGKIPAMVVDRCGCS

SEQ ID NO: 352 - protein, 112 aa, synthetic construct
  REGION 1..112: note = Synthetic
DFGLDCDEHSTESRCCRYPLTVDFEAFGWDWIIAPKRYKANYCSGECPYIWSLDTQYSKVLALYNQHNPGASAAPCCTPTKMSPINMLYFNGKEQIIYGKIPAMVVDRCGCS

SEQ ID NO: 353 - protein, 109 aa, synthetic construct
  REGION 1..109: note = Synthetic
DFGLDCDEHSTESRCCRYPLTVDFEAFGWDWIIAPKRYKANYCSGECEFVFLQKYPHTHLVHQANSVNSKIPCCTPTKMSPINMLYFNGKEQIIYGKIPAMVVDRCGCS

SEQ ID NO: 354 - protein, 109 aa, synthetic construct
  REGION 1..109: note = Synthetic
DFGLDCDEHSTESRCCRYPLTVDFEAFGWDWIIAPKRYKANYCSGECEFVFLQKYPHTHLVHQANPGASAAPCCTPTKMSPINMLYFNGKEQIIYGKIPAMVVDRCGCS

SEQ ID NO: 355 - protein, 109 aa, synthetic construct
  REGION 1..109: note = Synthetic
DFGLDCDEHSTESRCCRYPLTVDFEAFGWDWIIAPKRYKANYCSGECEFVFLQKYPHTHLVHQANPRGSAGPCCVPTELSAISMLYLDENEKVVLKNYQDMVVEGCGCS

SEQ ID NO: 356 - protein, 108 aa, synthetic construct
  REGION 1..108: note = Synthetic
DFGLDCDEHSTESRCCRYPLTVDFEAFGWDWIIAPKRYKANYCSGECEFVFLQKYPHTHLVHQANPRGSAGPCCVPQALEPLPIVYYVGRKPKVEQLSNMIVRSCGCS

SEQ ID NO: 357 - protein, 109 aa, synthetic construct
  REGION 1..109: note = Synthetic
DFGLDCDEHSTESRCCRYPLTVDFEAFGWDWIIAPKRYKANYCSGECEFVFLQKYPHTHLVHQANPRGSAGPCCTPTKMSPINMLYFNGKEKVVLKNYQDMVVEGCGCS

SEQ ID NO: 358 - protein, 108 aa, synthetic construct
  REGION 1..108: note = Synthetic
DFGLDCDEHSTESRCCRYPLTVDFEAFGWDWIIAPKRYKANYCSGECEFVFLQKYPHTHLVHQANPRGSAGPCCTPTKMSPINMLYFNGKKPKVEQLSNMIVRSCGCS

SEQ ID NO: 359 - DNA, 360 bp, synthetic construct
  misc_feature 1..360: note = Synthetic
  source 1..360: mol_type = other DNA
gaggtgcaggtgttggagtctgggggagacttggtacagcctggggggtccctgagactctcctgtgcagcctctggattcacctttagtgcctatgccatgacctgggtccgccaggctccagggaaggggctggagtgggtctcagctattagtggtagtggtggtagcgcatactacgcagactccgtgaagggccggttcaccatctccagagacaattccaagaacacggtatatctgcagatgaacagcctgagagccgaggacacggccgtatattactgtgcgaaagatggggcctggaaaatgtccggtttggacgtctggggccaagggaccacggtcatcgtctcctca

SEQ ID NO: 360 - protein, 120 aa, synthetic construct
  REGION 1..120: note = Synthetic
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 361 - DNA, 24 bp, synthetic construct
  misc_feature 1..24: note = Synthetic
  source 1..24: mol_type = other DNA
ggattcacctttagtgcctatgcc

SEQ ID NO: 362 - protein, 8 aa, synthetic construct
  REGION 1..8: note = Synthetic
GFTFSAYA

SEQ ID NO: 363 - DNA, 24 bp, synthetic construct
  misc_feature 1..24: note = Synthetic
  source 1..24: mol_type = other DNA
attagtggtagtggtggtagcgca

SEQ ID NO: 364 - protein, 8 aa, synthetic construct
  REGION 1..8: note = Synthetic
ISGSGGSA

SEQ ID NO: 365 - DNA, 39 bp, synthetic construct
  misc_feature 1..39: note = Synthetic
  source 1..39: mol_type = other DNA
gcgaaagatggggcctggaaaatgtccggtttggacgtc

SEQ ID NO: 366 - protein, 13 aa, synthetic construct
  REGION 1..13: note = Synthetic
AKDGAWKMSGLDV

SEQ ID NO: 367 - DNA, 321 bp, synthetic construct
  misc_feature 1..321: note = Synthetic
  source 1..321: mol_type = other DNA
gacatccagatgacccagtctccagcctccctgtctgcatctgttggagacagagtcaccatcacttgtcgggcgagtcaggacattagcgattatttagcctggtatcagcagaaaccagggaaaattcctaggctcctgatctatactacatccactttgcaatcaggggtcccatctcggttccgtggcagtgggtctgggacagatttcactctcaccatcagcagcctgcagcctgaagatgttgcaacttattactgtcagaagtatgacagtgccccgctcactttcggcggagggaccaaggtggagatcaaa

SEQ ID NO: 368 - protein, 107 aa, synthetic construct
  REGION 1..107: note = Synthetic
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 369 - DNA, 18 bp, synthetic construct
  misc_feature 1..18: note = Synthetic
  source 1..18: mol_type = other DNA
caggacattagcgattat

SEQ ID NO: 370 - protein, 6 aa, synthetic construct
  REGION 1..6: note = Synthetic
QDISDY

SEQ ID NO: 371-372 - skipped (2 entries)

SEQ ID NO: 373 - DNA, 27 bp, synthetic construct
  misc_feature 1..27: note = Synthetic
  source 1..27: mol_type = other DNA
cagaagtatgacagtgccccgctcact

SEQ ID NO: 374 - protein, 9 aa, synthetic construct
  REGION 1..9: note = Synthetic
QKYDSAPLT

SEQ ID NO: 375 - DNA, 363 bp, synthetic construct
  misc_feature 1..363: note = Synthetic
  source 1..363: mol_type = other DNA
caggtgcagctggtggagtctgggggaggcgtggtccagcctgggaagtccctgcgactctcctgtgcagcgtctggattcaccttcagtagttttggcatgcattgggtccgccaggctccaggcaaggggctggagtgggtggcagttattgggtatgatggaggtaatgaatactatgccgactccgtgaagggccgattcaccatctccagagacaattccaagaacacgctgaatctgcaaatgagcagcctgagagccgaagacacggctgtgtattattgttcgactataagtcattacgatattttgagcggtatggacgtctggggccgagggaccacggtcaccgtctcctca

SEQ ID NO: 376 - protein, 121 aa, synthetic construct
  REGION 1..121: note = Synthetic
QVQLVESGGGVVQPGKSLRLSCAASGFTFSSFGMHWVRQAPGKGLEWVAVIGYDGGNEYYADSVKGRFTISRDNSKNTLNLQMSSLRAEDTAVYYCSTISHYDILSGMDVWGRGTTVTVSS

SEQ ID NO: 377 - DNA, 24 bp, synthetic construct
  misc_feature 1..24: note = Synthetic
  source 1..24: mol_type = other DNA
ggattcaccttcagtagttttggc

SEQ ID NO: 378 - protein, 8 aa, synthetic construct
  REGION 1..8: note = Synthetic
GFTFSSFG

SEQ ID NO: 379 - DNA, 24 bp, synthetic construct
  misc_feature 1..24: note = Synthetic
  source 1..24: mol_type = other DNA
attgggtatgatggaggtaatgaa

SEQ ID NO: 380 - protein, 8 aa, synthetic construct
  REGION 1..8: note = Synthetic
IGYDGGNE

SEQ ID NO: 381 - DNA, 42 bp, synthetic construct
  misc_feature 1..42: note = Synthetic
  source 1..42: mol_type = other DNA
tcgactataagtcattacgatattttgagcggtatggacgtc

SEQ ID NO: 382 - protein, 14 aa, synthetic construct
  REGION 1..14: note = Synthetic
STISHYDILSGMDV

SEQ ID NO: 383 - DNA, 324 bp, synthetic construct
  misc_feature 1..324: note = Synthetic
  source 1..324: mol_type = other DNA
gacatccagatgacccagtctccatcttccgtgtctgcatctgtaggagacagagtcaccatcacttgtcgggcgagtcagggtattagcaactggttagcctggtatcagcagaaaccagggaaagcccctaagctcctgatctttgctgcatccagtttgcaaagtggggtcccatcacggttcagcggcagtgcatctgggacagatttcactctcaccatcaacagcctgcagcctgaagattttgcaacttactattgtcaacaggctaacagtttcccgctcactttcggcggagggaccaaggtggagatcaaacga

SEQ ID NO: 384 - protein, 108 aa, synthetic construct
  REGION 1..108: note = Synthetic
DIQMTQSPSSVSASVGDRVTITCRASQGISNWLAWYQQKPGKAPKLLIFAASSLQSGVPSRFSGSASGTDFTLTINSLQPEDFATYYCQQANSFPLTFGGGTKVEIKR

SEQ ID NO: 385 - DNA, 18 bp, synthetic construct
  misc_feature 1..18: note = Synthetic
  source 1..18: mol_type = other DNA
cagggtattagcaactgg

SEQ ID NO: 386 - protein, 6 aa, synthetic construct
  REGION 1..6: note = Synthetic
QGISNW

SEQ ID NO: 387-388 - skipped (2 entries)

SEQ ID NO: 389 - DNA, 27 bp, synthetic construct
  misc_feature 1..27: note = Synthetic
  source 1..27: mol_type = other DNA
caacaggctaacagtttcccgctcact

SEQ ID NO: 390 - protein, 9 aa, synthetic construct
  REGION 1..9: note = Synthetic
QQANSFPLT

SEQ ID NO: 391 - protein, 340 aa, synthetic construct
  REGION 1..340: note = Synthetic
EAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK